SEQUENCE LISTING (44 sequences)

SEQ ID NO: 1 - DNA, 1494 bp, Beta vulgaris
  source 1..1494: mol_type = other DNA
atggatcatgcaacattagcaatgatactagccatttggttcatttcttttcatttcataaaattactttttagccaacaaactaccaaacttcttcctcctggtccaaaaccattgccaataattggtaacatcttagaagttggtaaaaaaccccatcgttcatttgctaatcttgctaaaattcacggccctttaatatcgttacgtctaggaagtgtaacaactattgttgtatcatcagcagatgtagctaaagaaatgttcttaaaaaaagaccaccctctttctaaccgtactattcctaattctgtcacggccggtgaccaccataaactcaccatgtcgtggttgcccgtttcgccgaaatggcggaattttcgtaagattacagccgtccatttgctttctcctcaaagacttgatgcttgccaaacctttcgccatgccaaggtgcaacagctttatgaatatgtacaagaatgtgcacaaaaaggccaagctgttgatattggcaaagctgcatttactacctcccttaatttgttatctaaactattcttttcagtggaattagcccaccataaatcacacacttctcaagagtttaaggaactaatatggaacattatggaagatattgggaaacctaattatgctgattattttcctattttaggctgtgttgatccatcaggtattcgtcgaagattagcatgtagttttgacaagttgattgcagtttttcagggtataatatgtgaaaggcttgcgcctgattcttcaactacaacaacaacgacgactgatgatgtgctagacgttcttcttcagctcttcaaacaaaatgagctcactatgggcgagattaaccatttgctcgtcgacatttttgatgctggaacagacactacatcaagtacttttgagtgggtcatgacagagttaattaggaatcctgaaatgatggaaaaggctcaagaagaaattaagcaagtattgggcaaagataaacaaattcaggaatcagacattattaacctaccttacttacaagccattatcaaagaaactttgcgactacatccaccaactgtatttcttttgcctcgtaaagccgacactgatgttgaactatatggttatattgtgcctaaagatgcacaaatacttgttaacttatgggctattggaagagatcctaatgcatggcaaaatgctgatattttttcgcccgaaagatttatagggtgtgaaattgatgtcaaaggaagagattttggactcttaccttttggagccggaagaaggatatgtcctgggatgaatttggccattagaatgttaactttgatgctagctactttacttcaattcttcaattggaagcttgaaggagacataagtccaaaagacttagacatggatgagaaatttgggattgcgttacaaaagacaaagcctttaaaacttattccaatacctaggtattga

SEQ ID NO: 2 - protein, 497 aa, Beta vulgaris
MDHATLAMILAIWFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 3 - DNA, 800 bp, Mirabilis jalapa
  source 1..800: mol_type = other DNA
atgaaaggaacatactatataaatcatggtgatccactaatgtacttaaaaaaacatataaaactaaggcaattcttagaaggatggcaagaaaatgttgttattgaaaaaccaaagagtatacttatcatttctgctcattgggatactaatgtacctactgtcaactttgttgaacattgtgatactattcatgattttgatgactatcctgatcctttgtaccagatacaatatcgagcaccgggagcaccaaatttagcaaaaaaggtggaagagttactaaaagagtcaggaatggaatgtgagatagatacaaagagaggactcgatcatgcagcatggtttccactaatgtttatgtatcctgaagctaatattcctatttgtgagctctcagtccaaccaagcaaagatgggatccaccactataatgttgggaaagctctttctcctcttttgcaacaaggtgttctcatcattggctctggtggtactgttcatccttctgatgatactcctcattgtcccaatggtgttgctccttgggctattgagtttgataactggcttgaagatgctcttcttagtggaaggtatgaagatgtgaacaacttcaagaaattggcaccaaattgggagatatctcatccaggacaagaacatttgtaccctttgcatgtggcattaggggctgctggcaaaaatccaaagacacaacttattcatcgaagctgggctgccaatggtgtctttggatattccacctacaacttcactcccaccactcaaaaaactga

SEQ ID NO: 4 - protein, 267 aa, Mirabilis jalapa
MKGTYYINHGDPLMYLKKHIKLRQFLEGWQENVVIEKPKSILIISAHWDTNVPTVNFVEHCDTIHDFDDYPDPLYQIQYRAPGAPNLAKKVEELLKESGMECEIDTKRGLDHAAWFPLMFMYPEANIPICELSVQPSKDGIHHYNVGKALSPLLQQGVLIIGSGGTVHPSDDTPHCPNGVAPWAIEFDNWLEDALLSGRYEDVNNFKKLAPNWEISHPGQEHLYPLHVALGAAGKNPKTQLIHRSWAANGVFGYSTYNFTPTTQKTD

SEQ ID NO: 5 - DNA, 828 bp, Beta vulgaris
  source 1..828: mol_type = other DNA
atgaaaatgatgaatggtgaagatgctaatgatcaaatgatcaaagaaagcttcttcataacacatggaaacccaatattaacagtagaagacacacatccattaagacctttctttgaaacttggagagagaaaatcttttctaagaaacctaaggcaattcttattatttctggtcattgggaaactgttaaacctactgttaatgctgtccatatcaatgatactatccatgattttgatgactatcctgctgctatgtaccagttcaagtatccagctcctggggaaccagaattggcaagaaaagtagaggaaattctgaaaaaatcgggtttcgaaacggcggaaactgatcaaaaacgtgggcttgatcatggtgcatgggtacctctaatgctaatgtatcctgaggctgatataccagtatgtcagctctcagttcagccgcatttagatggaacataccattacaacttaggacgagcattggctcccttgaaaaacgacggcgtattaatcattggttcaggaagtgcaactcaccctttggatgaaactcctcattattttgatggagttgcaccttgggcagctgcctttgattcatggcttcgtaaagctctcattaatggaaggtttgaagaagtgaatatatatgaaagcaaagcaccaaattggaaattagcacatcctttcccagaacatttttatccattgcatgttgttcttggcgctgctggtgaaaaatggaaggcagagcttattcatagcagttgggatcatggcaccttgtgtcatggctcctacaagttcacttcagcctag

SEQ ID NO: 6 - protein, 275 aa, Beta vulgaris
MKMMNGEDANDQMIKESFFITHGNPILTVEDTHPLRPFFETWREKIFSKKPKAILIISGHWETVKPTVNAVHINDTIHDFDDYPAAMYQFKYPAPGEPELARKVEEILKKSGFETAETDQKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYESKAPNWKLAHPFPEHFYPLHVVLGAAGEKWKAELIHSSWDHGTLCHGSYKFTSA

SEQ ID NO: 7 - DNA, 2079 bp, Arabidopsis thaliana
  source 1..2079: mol_type = other DNA
atgacttctgctttgtatgcttccgatttgtttaagcagctcaagtcaattatggggacagattcgttatccgacgatgttgtacttgtgattgcaacgacgtctttggcactagtagctggatttgtggtgttgttatggaagaaaacgacggcggatcggagcggggagctgaagcctttgatgatccctaagtctcttatggctaaggacgaggatgatgatttggatttgggatccgggaagactagagtctctatcttcttcggtacgcagactggaacagctgagggatttgctaaggcattatccgaagaaatcaaagcgagatatgaaaaagcagcagtcaaagtcattgacttggatgactatgctgccgatgatgaccagtatgaagagaaattgaagaaggaaactttggcatttttctgtgttgctacttatggagatggagagcctactgacaatgctgccagattttacaaatggtttacggaggaaaatgaacgggatataaagcttcaacaactagcatatggtgtgtttgctcttggtaatcgccaatatgaacattttaataagatcgggatagttcttgatgaagagttatgtaagaaaggtgcaaagcgtcttattgaagtcggtctaggagatgatgatcagagcattgaggatgattttaatgcctggaaagaatcactatggtctgagctagacaagctcctcaaagacgaggatgataaaagtgtggcaactccttatacagctgttattcctgaataccgggtggtgactcatgatcctcggtttacaactcaaaaatcaatggaatcaaatgtggccaatggaaatactactattgacattcatcatccctgcagagttgatgttgctgtgcagaaggagcttcacacacatgaatctgatcggtcttgcattcatctcgagttcgacatatccaggacgggtattacatatgaaacaggtgaccatgtaggtgtatatgctgaaaatcatgttgaaatagttgaagaagctggaaaattgcttggccactctttagatttagtattttccatacatgctgacaaggaagatggctccccattggaaagcgcagtgccgcctcctttccctggtccatgcacacttgggactggtttggcaagatacgcagaccttttgaaccctcctcgaaagtctgcgttagttgccttggcggcctatgccactgaaccaagtgaagccgagaaacttaagcacctgacatcacctgatggaaaggatgagtactcacaatggattgttgcaagtcagagaagtcttttagaggtgatggctgcttttccatctgcaaaacccccactaggtgtattttttgctgcaatagctcctcgtctacaacctcgttactactccatctcatcctcgccaagattggcgccaagtagagttcatgttacatccgcactagtatatggtccaactcctactggtagaatccacaagggtgtgtgttctacgtggatgaagaatgcagttcctgcggagaaaagtcatgaatgtagtggagccccaatctttattcgagcatctaatttcaagttaccatccaacccttcaactccaatcgttatggtgggacctgggactgggctggcaccttttagaggttttctgcaggaaaggatggcactaaaagaagatggagaagaactaggttcatctttgctcttctttgggtgtagaaatcgacagatggactttatatacgaggatgagctcaataattttgttgatcaaggcgtaatatctgagctcatcatggcattctcccgtgaaggagctcagaaggagtatgttcaacataagatgatggagaaggcagcacaagtttgggatctaataaaggaagaaggatatctctatgtatgcggtgatgctaagggcatggcgagggacgtccaccgaactctacacaccattgttcaggagcaggaaggtgtgagttcgtcagaggcagaggctatagttaagaaacttcaaaccgaaggaagatacctcagagatgtctggtga

SEQ ID NO: 8 - protein, 692 aa, Arabidopsis thaliana [sequence omitted]

SEQ ID NO: 9 - DNA, 2325 bp, Arabidopsis thaliana
  source 1..2325: mol_type = other DNA
atgaaaaacatgatgaattataaattaaaactctgttctgtctcaaaaaactcaaaaggagtctctctctcacctacaccacacctaaccaaaccccctacgattcacacagagagagatcttcttcttccttcttcttccttcttctttcttcttctttcttcttctagctacaacatctacaacgccatgtcctcttcttcttcttcgtcaacctccatgatcgatctcatggcagcaatcatcaaaggagagcctgtaattgtctccgacccagctaatgcctccgcttacgagtccgtagctgctgaattatcctctatgcttatagagaatcgtcaattcgccatgattgttaccacttccattgctgttcttattggttgcatcgttatgctcgtttggaggagatccggttctgggaattcaaaacgtgtcgagcctcttaagcctttggttattaagcctcgtgaggaagagattgatgatgggcgtaagaaagttaccatctttttcggtacacaaactggtactgctgaaggttttgcaaaggctttaggagaagaagctaaagcaagatatgaaaagaccagattcaaaatcgttgatttggatgattacgcggctgatgatgatgagtatgaggagaaattgaagaaagaggatgtggctttcttcttcttagccacatatggagatggtgagcctaccgacaatgcagcgagattctacaaatggttcaccgaggggaatgacagaggagaatggcttaagaacttgaagtatggagtgtttggattaggaaacagacaatatgagcattttaataaggttgccaaagttgtagatgacattcttgtcgaacaaggtgcacagcgtcttgtacaagttggtcttggagatgatgaccagtgtattgaagatgactttaccgcttggcgagaagcattgtggcccgagcttgatacaatactgagggaagaaggggatacagctgttgccacaccatacactgcagctgtgttagaatacagagtttctattcacgactctgaagatgccaaattcaatgatataaacatggcaaatgggaatggttacactgtgtttgatgctcaacatccttacaaagcaaatgtcgctgttaaaagggagcttcatactcccgagtctgatcgttcttgtatccatttggaatttgacattgctggaagtggacttacgtatgaaactggagatcatgttggtgtactttgtgataacttaagtgaaactgtagatgaagctcttagattgctggatatgtcacctgatacttatttctcacttcacgctgaaaaagaagacggcacaccaatcagcagctcactgcctcctcccttcccaccttgcaacttgagaacagcgcttacacgatatgcatgtcttttgagttctccaaagaagtctgctttagttgcgttggctgctcatgcatctgatcctaccgaagcagaacgattaaaacaccttgcttcacctgctggaaaggatgaatattcaaagtgggtagtagagagtcaaagaagtctacttgaggtgatggccgagtttccttcagccaagccaccacttggtgtcttcttcgctggagttgctccaaggttgcagcctaggttctattcgatatcatcatcgcccaagattgctgaaactagaattcacgtcacatgtgcactggtttatgagaaaatgccaactggcaggattcataagggagtgtgttccacttggatgaagaatgctgtgccttacgagaagagtgaaaactgttcctcggcgccgatatttgttaggcaatccaacttcaagcttccttctgattctaaggtaccgatcatcatgatcggtccagggactggattagctccattcagaggattccttcaggaaagactagcgttggtagaatctggtgttgaacttgggccatcagttttgttctttggatgcagaaaccgtagaatggatttcatctacgaggaagagctccagcgatttgttgagagtggtgctctcgcagagctaagtgtcgccttctctcgtgaaggacccaccaaagaatacgtacagcacaagatgatggacaaggcttctgatatctggaatatgatctctcaaggagcttatttatatgtttgtggtgacgccaaaggcatggcaagagatgttcacagatctctccacacaatagctcaagaacaggggtcaatggattcaactaaagcagagggcttcgtgaagaatctgcaaacgagtggaagatatcttagagatgtatggtaa

SEQ ID NO: 10 - protein, 711 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 11 - DNA, 1503 bp, Mirabilis jalapa
  source 1..1503: mol_type = other DNA
atgaccgccattaaaatgaacaccaatggtgaaggtgaaacacaacatatactaatgatacctttcatggcgcaagggcatttacgtcctttccttgagcttgctatgtttctatataaacgaagtcatgttatcattactcttcttactaccccgctcaatgcgggtttcctacgacatctccttcaccaccatagctattctagctcggggatcagaattgtcgagttacctttcaactcaaccaatcatggtcttccacctggcattgaaaacactgataaacttacactcccacttgtagtatcactttttcattcaaccatttctcttgaccctcaccttagagattatatttcccgccatttctcccctgcgcgccctcctctgtgtgtcatacatgatgtgttccttggttgggttgatcaagttgctaaagacgtgggctcaactggtgttgtttttactacgggtggcgcgtatggtacaagcgcatatgtgtccatttggaatgatctgcctcaccagaattactctgatgatcaagagtttccgcttcctggtttcccggagaatcataaattccgacgttctcaacttcatcggtttctgaggtatgccgatggatcagatgattggtcgaaatatttccaaccgcaattgaggcaatcaatgaagagttttggatggctatgtaattcagttgaggaaatcgaaacacttgggtttagtatcctcaggaactacactaaactacccatttggggtattggaccgttgatagcttcacctgtacaacattcatcatctgataataacagtactggtgccgagtttgttcaatggttgagcttgaaagaaccagattctgtattatacatctcatttggatcacagaacacaatttcaccaacccagatgatggaactagcagccggtttggagtcaagtgagaagccgtttttgtgggtgattcgagcaccatttgggttcgatatcaatgaggaaatgagaccagaatggctaccagagggattcgaggagcgaatgaaggtgaaaaaacaaggaaagttggtgtataagttgggaccacagttggagatacttaaccatgagtcaatcggagggttcttaactcattgtgggtggaattcgatccttgagtcacttcgagaaggtgtgcctatgttagggtggccattggcagccgaacaagcttataatttgaagtatttggaggacgaaatgggggttgcagtcgagttagcgaggggattggaaggagagataagtaaagagaaagtgaagagaattgtggagatgattttagagagaaatgaaggaagtaaaggatgggaaatgaaaaatagagcagtagaaatggggaagaaacttaaagacgctgtcaatgaggagaaggaactgaagggttcttctgttaaggcaatagatgatttcttagatgcggtcatgcaagctaagttggaaccttctcttcaataa

SEQ ID NO: 12 - protein, 500 aa, Mirabilis jalapa
MTAIKMNTNGEGETQHILMIPFMAQGHLRPFLELAMFLYKRSHVIITLLTTPLNAGFLRHLLHHHSYSSSGIRIVELPFNSTNHGLPPGIENTDKLTLPLVVSLFHSTISLDPHLRDYISRHFSPARPPLCVIHDVFLGWVDQVAKDVGSTGVVFTTGGAYGTSAYVSIWNDLPHQNYSDDQEFPLPGFPENHKFRRSQLHRFLRYADGSDDWSKYFQPQLRQSMKSFGWLCNSVEEIETLGFSILRNYTKLPIWGIGPLIASPVQHSSSDNNSTGAEFVQWLSLKEPDSVLYISFGSQNTISPTQMMELAAGLESSEKPFLWVIRAPFGFDINEEMRPEWLPEGFEERMKVKKQGKLVYKLGPQLEILNHESIGGFLTHCGWNSILESLREGVPMLGWPLAAEQAYNLKYLEDEMGVAVELARGLEGEISKEKVKRIVEMILERNEGSKGWEMKNRAVEMGKKLKDAVNEEKELKGSSVKAIDDFLDAVMQAKLEPSLQ

SEQ ID NO: 13 - DNA, 363 bp, Saccharomyces cerevisiae
  source 1..363: mol_type = other DNA
atgcctaaagtttacagttaccaagaagttgccgaacacaatggcccagaaaatttctggattatcatcgatgacaaagtttacgatgtttctcaattcaaagatgaacatccaggtggtgatgaaattataatggatttgggtggacaagatgctacagaaagctttgtcgatatcggtcattctgacgaagcattgagactactgaaaggtttatacattggtgacgttgacaagaccagtgagcgcgtttctgtggaaaaggtatctacctctgaaaaccaaagtaaaggtagtggtacattggttgtcatattggccattttaatgctaggtgttgcttattatttgttgaacgaataa

SEQ ID NO: 14 - protein, 120 aa, Saccharomyces cerevisiae
MPKVYSYQEVAEHNGPENFWIIIDDKVYDVSQFKDEHPGGDEIIMDLGGQDATESFVDIGHSDEALRLLKGLYIGDVDKTSERVSVEKVSTSENQSKGSGTLVVILAILMLGVAYYLLNE

SEQ ID NO: 15 - DNA, 1470 bp, Dorotheanthus bellidiformis
  source 1..1470: mol_type = other DNA
atgggtacacactcaacagcaccagatctccatgtagttttcttcccattcttggctcatggccacatgatcccgtccctagacatcgcgaagctctttgcagctcgcggtgtcaaaacaaccatcatcaccacccctcttaacgcatccatgttcaccaaagcaatagaaaaaaccagaaaaaacacagaaactcagatggaaattgaagttttcagcttcccgtctgaagaagctggtttgccccttggatgtgagaatttggagcaagccatggctattggtgcaaataatgaattctttaatgctgcgaatttgctgaaagagcagctggagaattttctagtgaaaactcgaccaaattgccttgtggccgatatgttcttcacctgggcagctgattccacagccaaattcaacatccctaccctagtttttcatggatttagctttttcgcccaatgtgccaaagaggttatgtggaggtataagccttataaggccgtctcatccgacacagaggtattttctcttccatttcttccccatgaggtgaagatgaccagattgcaggttccagagagtatgaggaaaggtgaggaaactcacttcactaagagaacagaacgcatcagggaattggaacgcaagagttatggggtgattgttaacagcttttacgagttggagcccgattatgcagactttcttaggaaagaattggggagaagggcatggcatataggccctgtttcactatgcaataggagcatcgaggataaagctcaacgagggagacaaacttcaatcgatgaagatgaatgcttgaaatggcttaactccaaaaaacccgattcagttatctatatctgtttcggaagcacaggacacctcatagcccctcagttacacgagattgcaacggctctagaagcttccggacaagactttatctgggcggtgaggggtgatcatggtcaaggaaatagcgaagagtggttgccaccgggatacgaacatagattgcaagggaaaggcttgataatacgagggtgggctccacaggtgttgattttggagcacgaagctacaggaggatttctgactcactgtgggtggaactcggcacttgaagggatctctgctggtgtgccaatggttacatggcctacttttgccgagcaattccataacgagcagttgttgacacagattttgaaggtcggggtcgctgttggatctaagaagtggaccctaaagccttctatcgaagatgtgataaaagcggaagatatagagaaggcagttagagaagttatggtgggagaggaaggtgaagaaaggagaaggagggcaaagaagttgaaggaaatggcatggagggctattgaagaaggtggatcatcctactcggatttaagtgcactgattgaggaattgaagggttatcatacttcagagaaagagtag

SEQ ID NO: 16 - protein, 489 aa, Dorotheanthus bellidiformis
MGTHSTAPDLHVVFFPFLAHGHMIPSLDIAKLFAARGVKTTIITTPLNASMFTKAIEKTRKNTETQMEIEVFSFPSEEAGLPLGCENLEQAMAIGANNEFFNAANLLKEQLENFLVKTRPNCLVADMFFTWAADSTAKFNIPTLVFHGFSFFAQCAKEVMWRYKPYKAVSSDTEVFSLPFLPHEVKMTRLQVPESMRKGEETHFTKRTERIRELERKSYGVIVNSFYELEPDYADFLRKELGRRAWHIGPVSLCNRSIEDKAQRGRQTSIDEDECLKWLNSKKPDSVIYICFGSTGHLIAPQLHEIATALEASGQDFIWAVRGDHGQGNSEEWLPPGYEHRLQGKGLIIRGWAPQVLILEHEATGGFLTHCGWNSALEGISAGVPMVTWPTFAEQFHNEQLLTQILKVGVAVGSKKWTLKPSIEDVIKAEDIEKAVREVMVGEEGEERRRRAKKLKEMAWRAIEEGGSSYSDLSALIEELKGYHTSEKE

SEQ ID NO: 17 - DNA, 1500 bp, Saccharomyces cerevisiae
  source 1..1500: mol_type = other DNA
atgtccactaagaagcacaccaaaacacattccacttatgcattcgagagcaacacaaacagcgttgctgcctcacaaatgagaaacgccttaaacaagttggcggactctagtaaacttgacgatgctgctcgcgctaagtttgagaacgaactggattcgtttttcacgcttttcaggagatatttggtagagaagtcttctagaaccaccttggaatgggacaagatcaagtctcccaacccggatgaagtggttaagtatgaaattatttctcagcagcccgagaatgtctcaaacctttccaaattggctgttttgaagttgaacggtgggctgggcacctccatgggctgcgttggccctaaatctgttattgaagtgagagagggaaacacctttttggatttgtctgttcgtcaaattgaatacttgaacagacagtacgatagcgacgtgccattgttattgatgaattctttcaacactgacaaggatacggaacacttgattaagaagtattccgctaacagaatcagaatcagatctttcaatcaatccaggttcccaagagtctacaaggattctttattgcctgtccccaccgaatacgattctccactggatgcttggtatccaccaggtcacggtgatttgtttgaatctttacacgtatctggtgaactggatgccttaattgcccaaggaagagaaatattatttgtttctaacggtgacaacttgggtgctaccgtcgacttaaaaattttaaaccacatgatcgagactggtgccgaatatataatggaattgactgataagaccagagccgatgttaaaggtggtactttgatttcttacgatggtcaagtccgtttattggaagtcgcccaagttccaaaagaacacattgacgaattcaaaaatatcagaaagtttaccaacttcaacacgaataacttatggatcaatctgaaagcagtaaagaggttgatcgaatcgagcaatttggagatggaaatcattccaaaccaaaaaactataacaagagacggtcatgaaattaatgtcttacaattagaaaccgcttgtggtgctgctatcaggcattttgatggtgctcacggtgttgtcgttccaagatcaagattcttgcctgtcaagacctgttccgatttgttgctggttaaatcagatctattccgtctggaacacggttctttgaagttagacccatcccgttttggtccaaacccattaatcaagttgggctcgcatttcaaaaaggtttctggttttaacgcaagaatccctcacatcccaaaaatcgtcgagctagatcatttgaccatcactggtaacgtctttttaggtaaagatgtcactttgaggggtactgtcatcatcgtttgctccgacggtcataaaatcgatattccaaacggctccatattggaaaatgttgtcgttactggtaatttgcaaatcttggaacattga

SEQ ID NO: 18 - protein, 499 aa, Saccharomyces cerevisiae
MSTKKHTKTHSTYAFESNTNSVAASQMRNALNKLADSSKLDDAARAKFENELDSFFTLFRRYLVEKSSRTTLEWDKIKSPNPDEVVKYEIISQQPENVSNLSKLAVLKLNGGLGTSMGCVGPKSVIEVREGNTFLDLSVRQIEYLNRQYDSDVPLLLMNSFNTDKDTEHLIKKYSANRIRIRSFNQSRFPRVYKDSLLPVPTEYDSPLDAWYPPGHGDLFESLHVSGELDALIAQGREILFVSNGDNLGATVDLKILNHMIETGAEYIMELTDKTRADVKGGTLISYDGQVRLLEVAQVPKEHIDEFKNIRKFTNFNTNNLWINLKAVKRLIESSNLEMEIIPNQKTITRDGHEINVLQLETACGAAIRHFDGAHGVVVPRSRFLPVKTCSDLLLVKSDLFRLEHGSLKLDPSRFGPNPLIKLGSHFKKVSGFNARIPHIPKIVELDHLTITGNVFLGKDVTLRGTVIIVCSDGHKIDIPNGSILENVVVTGNLQILEH

SEQ ID NO: 19 - DNA, 1710 bp, Saccharomyces cerevisiae
  source 1..1710: mol_type = other DNA
atgtcatttcaaattgaaacggttcccaccaaaccatatgaagaccaaaagcctggtacttctggtttgcgtaagaagacaaaggtgtttaaagacgaacctaactacacagaaaatttcattcaatcgatcatggaagctattccagagggttctaaaggtgccactcttgttgtcggtggtgatgggcgttactacaatgatgtcattcttcataagattgccgctatcggtgctgccaacggtattaaaaagttagttattggccagcatggtcttctgtctacgccagccgcttctcacatcatgagaacctacgaggaaaaatgtactggtggtattatcttaaccgcctcacataatccaggtggtccagaaaatgacatgggtattaagtataacttatccaatgggggtcctgctcctgaatccgtcacaaatgctatttgggagatttccaaaaagcttaccagctataagattatcaaagacttcccagaactagacttgggtacgataggcaagaacaagaaatacggtccattactcgttgacattatcgatattacaaaagattatgtcaacttcttgaaggaaatcttcgatttcgacttaatcaagaaattcatcgataatcaacgttctactaagaattggaagttactgtttgacagtatgaacggtgtaactggaccatacggtaaggctattttcgttgatgaatttggtttaccggcggatgaggttttacaaaactggcatccttctccggattttggtggtatgcatccagatccaaacttaacttatgccagttcgttagtgaaaagagtagatcgtgaaaagattgagtttggtgctgcatccgatggtgatggtgatagaaatatgatttacggttacggcccatctttcgtttctccaggtgactccgtcgcaattattgccgaatatgcagctgaaatcccatatttcgccaagcaaggtatatatggtctggcccgttcattccctacctcaggagccatagaccgtgttgccaaggcccatggtctaaactgttatgaggtcccaactggctggaaatttttctgtgctttgttcgacgctaaaaaattatctatttgtggtgaagaatcgtttggtactggttccaaccacgtaagggaaaaggacggtgtttgggccattatggcgtggttgaacatcttggccatttacaacaagcatcatccggagaacgaagcttctattaagacgatacagaatgaattctgggcaaagtacggccgtactttcttcactcgttatgattttgaaaaagttgaaacagaaaaagctaacaagattgtcgatcaattgagagcatatgttaccaaatcgggtgttgttaattccgccttcccagccgatgagtctcttaaggtcaccgattgtggtgatttttcatacacagatttggacggttctgtttctgaccatcaaggtttatatgtcaagctttccaatggtgcaagattcgttctaagattgtcaggtacaggttcttcaggtgctaccattagattgtacattgaaaaatactgcgatgataaatcacaataccaaaagacagctgaagaatacttgaagccaattattaactcggtcatcaagttcttgaactttaaacaagttttaggaactgaagaaccaacggttcgtacttaa

SEQ ID NO: 20 - protein, 569 aa, Saccharomyces cerevisiae
MSFQIETVPTKPYEDQKPGTSGLRKKTKVFKDEPNYTENFIQSIMEAIPEGSKGATLVVGGDGRYYNDVILHKIAAIGAANGIKKLVIGQHGLLSTPAASHIMRTYEEKCTGGIILTASHNPGGPENDMGIKYNLSNGGPAPESVTNAIWEISKKLTSYKIIKDFPELDLGTIGKNKKYGPLLVDIIDITKDYVNFLKEIFDFDLIKKFIDNQRSTKNWKLLFDSMNGVTGPYGKAIFVDEFGLPADEVLQNWHPSPDFGGMHPDPNLTYASSLVKRVDREKIEFGAASDGDGDRNMIYGYGPSFVSPGDSVAIIAEYAAEIPYFAKQGIYGLARSFPTSGAIDRVAKAHGLNCYEVPTGWKFFCALFDAKKLSICGEESFGTGSNHVREKDGVWAIMAWLNILAIYNKHHPENEASIKTIQNEFWAKYGRTFFTRYDFEKVETEKANKIVDQLRAYVTKSGVVNSAFPADESLKVTDCGDFSYTDLDGSVSDHQGLYVKLSNGARFVLRLSGTGSSGATIRLYIEKYCDDKSQYQKTAEEYLKPIINSVIKFLNFKQVLGTEEPTVRT

SEQ ID NO: 21 - DNA, 462 bp, Saccharomyces cerevisiae
  source 1..462: mol_type = other DNA
atgtctagtcaaacagaaagaacttttattgcggtaaaaccagatggtgtccagaggggcttagtatctcaaattctatctcgttttgaaaaaaaaggttacaaactagttgctattaaattagttaaagcggatgataaattactagagcaacattacgcagagcatgttggtaaaccatttttcccaaagatggtatcctttatgaagtctggtcccattttggccacggtctgggagggaaaagatgtggttagacaaggaagaactattcttggtgctactaatcctttgggcagtgcaccaggtaccattagaggtgatttcggtattgacctaggcagaaacgtctgtcacggcagtgattctgttgatagcgctgaacgtgaaatcaatttgtggtttaagaaggaagagttagttgattgggaatctaatcaagctaagtggatttatgaatga

SEQ ID NO: 22 - protein, 153 aa, Saccharomyces cerevisiae
MSSQTERTFIAVKPDGVQRGLVSQILSRFEKKGYKLVAIKLVKADDKLLEQHYAEHVGKPFFPKMVSFMKSGPILATVWEGKDVVRQGRTILGATNPLGSAPGTIRGDFGIDLGRNVCHGSDSVDSAEREINLWFKKEELVDWESNQAKWIYE

SEQ ID NO: 23 - DNA, 1347 bp, Saccharomyces cerevisiae
  source 1..1347: mol_type = other DNA
atgctttcgcttaaaacgttactgtgtacgttgttgactgtgtcatcagtactcgctaccccagtccctgcaagagacccttcttccattcaatttgttcatgaggagaacaagaaaagatactacgattatgaccacggttccctcggagaaccaatccgtggtgtcaacattggtggttggttacttcttgaaccatacattactccatctttgttcgaggctttccgtacaaatgatgacaacgacgaaggaattcctgtcgacgaatatcacttctgtcaatatttaggtaaggatttggctaaaagccgtttacagagccattggtctactttctaccaagaacaagatttcgctaatattgcttcccaaggtttcaaccttgtcagaattcctatcggttactgggctttccaaactttggacgatgatccttatgttagcggcctacaggaatcttacctagaccaagccatcggttgggctagaaacaacagcttgaaagtttgggttgatttgcatggtgccgctggttcgcagaacgggtttgataactctggtttgagagattcatacaagtttttggaagacagcaatttggccgttactacaaatgtcttgaactacatattgaaaaaatactctgcggaggaatacttggacactgttattggtatcgaattgattaatgagccattgggtcctgttctagacatggataaaatgaagaatgactacttggcacctgcttacgaatacttgagaaacaacatcaagagtgaccaagttatcatcatccatgacgctttccaaccatacaattattgggatgacttcatgactgaaaacgatggctactggggtgtcactatcgaccatcatcactaccaagtctttgcttctgatcaattggaaagatccattgatgaacatattaaagtagcttgtgaatggggtaccggagttttgaatgaatcccactggactgtttgtggtgagtttgctgccgctttgactgattgtacaaaatggttgaatagtgttggcttcggcgctagatacgacggttcttgggtcaatggtgaccaaacatcttcttacattggctcttgtgctaacaacgatgatatagcttactggtctgacgaaagaaaggaaaacacaagacgttatgtggaggcacaactagatgcctttgaaatgagagggggttggattatctggtgttacaagacagaatctagtttggaatgggatgctcaaagattgatgttcaatggtttattccctcaaccattgactgacagaaagtatccaaaccaatgtggcacaatttctaactaa

SEQ ID NO: 24 - protein, 448 aa, Saccharomyces cerevisiae
MLSLKTLLCTLLTVSSVLATPVPARDPSSIQFVHEENKKRYYDYDHGSLGEPIRGVNIGGWLLLEPYITPSLFEAFRTNDDNDEGIPVDEYHFCQYLGKDLAKSRLQSHWSTFYQEQDFANIASQGFNLVRIPIGYWAFQTLDDDPYVSGLQESYLDQAIGWARNNSLKVWVDLHGAAGSQNGFDNSGLRDSYKFLEDSNLAVTTNVLNYILKKYSAEEYLDTVIGIELINEPLGPVLDMDKMKNDYLAPAYEYLRNNIKSDQVIIIHDAFQPYNYWDDFMTENDGYWGVTIDHHHYQVFASDQLERSIDEHIKVACEWGTGVLNESHWTVCGEFAAALTDCTKWLNSVGFGARYDGSWVNGDQTSSYIGSCANNDDIAYWSDERKENTRRYVEAQLDAFEMRGGWIIWCYKTESSLEWDAQRLMFNGLFPQPLTDRKYPNQCGTISN

SEQ ID NO: 25 - DNA, 1893 bp, Saccharomyces cerevisiae
  source 1..1893: mol_type = other DNA
atgctcatagaaaacactaatgatcggtttggtatcgtcatagatgcagggtcttcgggttcgagaatccatgtgtttaagtggcaggacacggaatcgcttcttcatgcaacaaaccaggactcacagtccatattacaatcagtacctcatattcatcaagaaaaagactggacttttaagctgaatccaggcttgtcgagttttgaaaaaaaacctcaagatgcgtacaaatctcacatcaagccgttactagattttgctaagaatattatccctgaatcacattggtcgagttgtcccgtttttattcaagcaaccgcgggcatgcgccttttacctcaagacatacaatcttccattttggatggtttgtgtcaggggctcaaacaccctgcagaatttttggttgaggattgctcagcacaaattcaagtcattgatggtgaaaccgaaggtttatatggctggcttggcttaaactatctatatggacactttaatgactataatccagaggtctctgaccattttacatttgggtttatggacatgggcggtgcctctactcagattgcgtttgcaccgcatgattcaggagaaatagctagacatagagatgacatcgccaccatcttcttgaggagtgttaacggagatttgcagaaatgggacgtttttgttagtacgtggttagggtttggtgccaatcaggctagaagaaggtacttggctcagttgatcaatacccttccagaaaacacaaatgattacgaaaatgatgacttctctacaaggaacttgaatgatccatgtatgccgcgaggaagcagcacggattttgaatttaaagataccatatttcacatcgcgggttctgggaattacgaacaatgtacaaaatctatttatccgttacttttaaagaacatgccttgtgatgacgagccgtgcttgtttaatggtgttcatgctcctcgaatagactttgccaatgacaaatttataggtacttctgagtactggtacactgccaacgacgtattcaaacttgggggcgaatacaactttgacaaatttagcaaaagcctaagggagttttgcaattccaattggacgcaaatattagcgaacagtgataaaggcgtgtataattctattccggagaattttttgaaagatgcatgctttaagggcaattgggtccttaatatacttcatgaagggtttgatatgcctcgaatagatgtcgacgcagaaaatgtcaatgacaggcccttatttcaaagtgtggaaaaagtcgaagaacgagagttatcgtggacactaggcagaattttactttatgcctcaggtagcatattggcaggtaatgatgacttcatggtgggtattgcgcccagtgaaagaagaactaagctcactggtaagaaattcatacctggcaagttactggagtctgatcaactacgcaagcaaagttccagcctttctaataaaggatttttgatgtggttcgcaattatttgttgcatattttacttgatctttcataggtcacacataatcagaagacgtttttccggtctgtacaacattaccaaggactttaagacaggcataaggagaaggttgaaatttctaaggagatcagatccattttccagattggaggaaggtgaacttggaacagacgtagatggcttcaaagatgtgtacaggatgaagagtagcagtatgtttgatcttggtaagagttcagccacaatgcaaagggagcacgaaccacaaaggacagcaagtcagtccgctaatctcgctccgtcaaacttacgacctgcgttttctatggctgatttttccaaatttaaggacagtaggctatatgattga

SEQ ID NO: 26 - protein, 630 aa, Saccharomyces cerevisiae
MLIENTNDRFGIVIDAGSSGSRIHVFKWQDTESLLHATNQDSQSILQSVPHIHQEKDWTFKLNPGLSSFEKKPQDAYKSHIKPLLDFAKNIIPESHWSSCPVFIQATAGMRLLPQDIQSSILDGLCQGLKHPAEFLVEDCSAQIQVIDGETEGLYGWLGLNYLYGHFNDYNPEVSDHFTFGFMDMGGASTQIAFAPHDSGEIARHRDDIATIFLRSVNGDLQKWDVFVSTWLGFGANQARRRYLAQLINTLPENTNDYENDDFSTRNLNDPCMPRGSSTDFEFKDTIFHIAGSGNYEQCTKSIYPLLLKNMPCDDEPCLFNGVHAPRIDFANDKFIGTSEYWYTANDVFKLGGEYNFDKFSKSLREFCNSNWTQILANSDKGVYNSIPENFLKDACFKGNWVLNILHEGFDMPRIDVDAENVNDRPLFQSVEKVEERELSWTLGRILLYASGSILAGNDDFMVGIAPSERRTKLTGKKFIPGKLLESDQLRKQSSSLSNKGFLMWFAIICCIFYLIFHRSHIIRRRFSGLYNITKDFKTGIRRRLKFLRRSDPFSRLEEGELGTDVDGFKDVYRMKSSSMFDLGKSSATMQREHEPQRTASQSANLAPSNLRPAFSMADFSKFKDSRLYD

SEQ ID NO: 27 - DNA, 615 bp, Saccharomyces cerevisiae
  source 1..615: mol_type = other DNA
atgacagctgccactacatcacagccagctttctcgcctgaccaagtttccgtgatcttcgttctaggaggacccggtgcaggcaagggtactcagtgtgaaaaactagttaaggactattcatttgtccatttgtcagccggagaccttctacgtgctgagcagggcagagcaggttcccaatatggggaattgatcaagaactgcatcaaagagggccagattgtccctcaagagattactttggcgcttttacgcaacgctatttccgataacgtcaaggcgaacaagcataagttcttaattgacggatttcctaggaagatggatcaagccatttcctttgaaagagacatcgttgaaagcaaattcatcctgttctttgactgccctgaagatatcatgttagagagactattggagcgtggcaagaccagtggtagaagcgatgacaacattgagtccattaagaagagatttaacactttcaaggagactagtatgcccgtcatcgagtactttgaaaccaaatcgaaagtcgtccgtgttcgttgcgacagatccgtcgaagatgtgtacaaagacgtccaagacgctatccgtgatagcttatag

SEQ ID NO: 28 - protein, 204 aa, Saccharomyces cerevisiae
MTAATTSQPAFSPDQVSVIFVLGGPGAGKGTQCEKLVKDYSFVHLSAGDLLRAEQGRAGSQYGELIKNCIKEGQIVPQEITLALLRNAISDNVKANKHKFLIDGFPRKMDQAISFERDIVESKFILFFDCPEDIMLERLLERGKTSGRSDDNIESIKKRFNTFKETSMPVIEYFETKSKVVRVRCDRSVEDVYKDVQDAIRDSL

SEQ ID NO: 29 - DNA, 2079 bp, synthetic construct
  source 1..2079: mol_type = other DNA
atgacctctgctttgtacgcttccgatttgtttaagcagctgaaatcaattatggggacagattcgttaagcgacgacgttgtacttgttattgcaacgacgtctttggcactggtagctggatttgtggtgttgttatggaagaaaaccacagctgataggagcggggaactgaagcctttgatgatccctaaatctcttatggctaaagacgaggatgatgatttggatttgggatctggtaagactagagtctctatcttcttcggtacgcagactggaacagctgagggttttgctaaagcattatccgaagaaatcaaagcgagatatgaaaaagcagcagtcaaagtcattgacttggatgactatgctgccgatgatgaccagtatgaagagaaattgaagaaagaaactttggcatttttctgtgttgctacctatggagatggagaacctactgacaatgctgccagattctacaaatggtttacggaggaaaatgaaagagacataaagcttcaacaactagcatacggtgtctttgctcttggtaatcgccaatatgaacatttcaataaaatcgggatagttttagatgaagagttatgtaaaaaaggtgcaaaacgtcttattgaagtcggtctaggagatgatgatcagagcattgaggatgattttaatgcctggaaagaatcactatggtctgagttagacaagctcctgaaagacgaagatgataaaagtgtcgcaactccgtatacagctgttattcctgaataccgggtggtcactcatgatcctcgctttacaactcaaaaatcaatggaatcaaacgtggccaatggaaacacaactattgacattcatcatccctgtagagttgatgttgctgtgcagaaggagcttcacacacatgaatctgataggtcttgcattcatttagaattcgacattagcaggacgggtattacatatgaaacaggtgaccatgtaggtgtatatgctgaaaatcatgttgaaatagttgaagaagctggaaaattgttaggccactctttagatttagtattttccatacatgctgacaaggaagatggctccccattggaaagcgcagtgccgccgcccttcccgggtccatgcacattagggactggtttggccagatacgcagacttattgaacccaccaagaaagagcgcgttagttgccttggcggcctatgccactgaaccaagtgaagccgagaaacttaagcacctgacatcacccgatggcaaggatgagtactcacaatggattgttgccagtcaaagaagtcttttagaagtgatggctgcttttccatctgcaaaacccccactaggtgtattctttgctgccatagctccccgtctacaacctcgttactactccatctcatcgtcgccaagattggcgccaagtagagttcatgttacatccgcactagtatatggtccaacccctactggtagaatccacaagggtgtctgttctacctggatgaagaacgcagtacctgcggagaaaagtcatgaatgtagtggagccccaatctttattcgagcgtctaatttcaagttaccatccaacccttcaactccaatcgttatggtgggaccaggcaccggtctggccccttttagaggttttctgcaagaaaggatggccctaaaagaagatggcgaagaattaggttcatctttgctcttctttgggtgtagaaatcgacaaatggactttatatacgaggatgaactcaacaactttgttgatcaaggcgtaatatctgagttaataatggcattctcgcgtgaaggcgctcaaaaggagtatgttcaacataagatgatggaaaaagcagcacaagtttgggatctaataaaagaagaaggttatctctatgtatgcggtgatgctaagggcatggcgagggacgtccacaggactctacacaccattgttcaggagcaggaaggtgtgagttcgtcagaagcagaggctattgttaagaaattacaaaccgaaggcagatacttgagagatgtctggtaa

SEQ ID NO: 30 - DNA, 2136 bp, synthetic construct
  source 1..2136: mol_type = other DNA
atgtcttcctccagcagttcatcaacatccatgatcgatcttatggcagcaataataaaaggcgaacccgttatagtgagtgacccagcaaacgcgtccgcgtatgaaagcgtcgcggcggagctatcctccatgttgatagagaataggcaatttgcaatgatcgtgacaacatcaatcgcggtcttgattgggtgtattgtaatgctagtatggcgtcgttctggtagtggtaactctaaaagagtagaaccgttaaaacccctggtcataaaaccacgtgaggaggaaatagacgatgggcgtaaaaaggttacaatattcttcggcacacaaaccggtacagcggagggctttgcaaaggcccttggtgaagaagcgaaggcgaggtacgaaaagaccagattcaaaatcgttgacctggacgattatgctgctgacgatgatgaatacgaggaaaaattaaaaaaagaggacgtggccttcttctttcttgctacctatggggacggcgaacctaccgacaacgccgcaagattctataagtggttcaccgaaggtaacgatcgtggagaatggctaaagaatctgaagtacggtgtcttcggtctaggaaacaggcagtatgaacacttcaacaaggtagcgaaagttgtagatgacattctggttgagcagggtgcgcagagactggtacaggttggactaggcgacgatgaccaatgcattgaggacgactttactgcgtggagggaggcattatggcctgagctggatacgatcctacgtgaagaaggagacacggccgttgctactccctacacagcagctgttttagagtatagagtgtccatccatgattccgaggatgccaagttcaacgacataaacatggcaaatggcaacggatacactgtttttgacgctcagcatccgtacaaagccaacgtagcagtcaaaagagagctgcatacccccgaaagcgaccgtagttgtatccacttggagttcgatatagccggctccggattaacttacgaaaccggtgatcacgtcggagtgttgtgtgacaatttatccgagaccgtagatgaggccttgaggttgctggatatgagccccgacacgtatttttccctgcatgccgagaaggaagatgggacgccgattagctccagcctgccgccgccctttcctccttgcaatctaagaacggctctaacgcgttatgcgtgtctattatcttctccgaagaagagtgcgttggtggcgctggcggcgcatgcgagcgatccgacggaagcagaaagactaaagcatctggcttctcccgctggcaaggacgagtactctaagtgggtggtagaaagtcagagatcattattagaggtgatggctgaatttccgagcgctaaaccgccgcttggcgtttttttcgcaggggtcgcacctcgtttacagccaaggttctactccattagcagctccccaaaaatagctgaaacaagaatccacgtaacgtgtgctctggtctatgagaagatgccgacgggcagaatccataaaggggtgtgttccacttggatgaaaaatgcggtgccctatgaaaagtccgagaattgttcatctgcccccattttcgtcaggcagtccaatttcaaacttccatccgactcaaaagtcccgataatcatgataggccctgggacgggtctagctccgttcagaggttttttacaagagcgtctagctcttgtggagtcaggggtggagttgggtcccagtgtcctgttttttggttgtaggaacaggaggatggattttatctacgaggaagaattgcaacgttttgttgaatccggagcactagcggaattgtcagtcgctttttcaagagaaggtccgacaaaagagtacgtacaacacaagatgatggacaaggcttcagatatttggaatatgatatctcagggcgcatacctttatgtgtgcggtgacgctaagggtatggcaagggacgtccataggagtttacacaccattgcacaagaacaagggagcatggacagtaccaaggcagaagggtttgtgaagaatttgcaaacgagcggaaggtatctgcgtgacgtttggtaa

SEQ ID NO: 31 - DNA, 1500 bp, synthetic construct
  source 1..1500: mol_type = other DNA
atgtccactaagaagcacaccaaaacacatgctacttatgcattcgagagcaacacaaacagcgttgctgcctcacaaatgagaaacgccttaaacaagttggcggactctagtaaacttgacgatgctgctcgcgctaagtttgagaacgaactggattcgtttttcacgcttttcaggagatatttggtagagaagtcttcaagaaccaccttggaatgggacaagatcaagtctcccaacccggatgaagtggttaagtatgaaattatttctcagcagcccgagaatgtctcaaacctttccaaattggctgttttgaagttgaacggtgggctgggcacctctatgggctgcgttggccctaaatctgttattgaagtgagagagggaaacacctttttggatttgtctgttcgtcaaattgaatacttgaacagacagtacgatagcgacgtgccattgttattgatgaattttttcaacactgacaaggatacggaacacttgattaagaagtattccgctaacagaatcagaatcagatctttcaatcaatccaggttcccaagagtctacaaggattctttattgcctgtccccaccgaatacgattctccactggatgcttggtatccaccaggtcacggtgatttgtttgaatctttacacgtatctggtgaactggatgccttaattgcccaaggaagagaaatattatttgtttctaacggtgacaacttgggtgctaccgttgacttaaaaattttaaaccacatgatcgagactggtgccgaatatataatggaattgactgataagaccagagccgatgttaaaggtggtactttgatttcttacgatggtcaagtccgtttattggaagtcgcccaagttccaaaagaacacattgacgaatttaaaaatatcagaaagtttaccaacttcaacacgaataacttatggatcaatctgaaagcagtaaagaggttgatcgaatcgagcaatttggagatggaaatcattccaaaccaaaaaactataacaagagacggtcatgaaattaatgtcttacaattagaaaccgcttgtggtgctgctatcaggcattttgatggtgctcacggtgttgtcgttccaagatcaagattcttgcctgtcaagacctgttccgatttgttgctggttaaatcagatctattccgtctggaacacggttctttgaagttagacccatcccgttttggtccaaacccattaatcaagttgggctcgcatttcaaaaaggtttctggttttaacgcaagaatccctcacatcccaaaaatcgtcgagctagatcatttgaccatcactggtaacgtctttttaggtaaagatgtcactttgaggggtactgtcatcatcgtttgctccgacggtcataaaatcgatattccaaacggctccatattggaaaatgttgtcgttactggtaatttgcaaatcttggaacattga

SEQ ID NO: 32 - protein, 498 aa, synthetic construct
MSTKKHTKTHATYAFESNTNSVAASQMRNALNKLADSSKLDDAARAKFENELDSFFTLFRRYLVEKSSRTTLEWDKIKSPNPDEVVKYEIISQQPENVSNLSKLAVLKLNGGLGTSMGCVGPKSVIEVREGNTFLDLSVRQIEYLNRQYDSDVPLLLMNFFNTDKDTEHLIKKYSANRIRIRSFNQSRFPRVYKDSLLPVPTEYDSPLDAWYPPGHGDLFESLHVSGELDALIAQGREILFVSNGDNLGATVDLKILNHMIETGAEYIMELTDKTRADVKGGTLISYDGQVRLLEVAQVPKEHIDEFKNIRKFTNFNTNNLWINLKAVKRLIESSNLEMEIIPNQKTITRDGHEINVLQLETACGAAIRHFDGAHGVVVPRSRFLPVKTCSDLLLVKSDLFRLEHGSLKLDPSRFGPNPLIKLGSHFKKVSGFNARIPHIPKIVELDHLTITGNVFLGKDVTLRGTVIIVCSDGHKIDIPNGSILENVVVTGNLQILE

SEQ ID NO: 33 - DNA, 1710 bp, synthetic construct
  source 1..1710: mol_type = other DNA
atgtcatttcaaattgaaacggttcccaccaaaccttatgaagaccaaaagcctggtacttctggtttgcgtaagaagacaaaggtgtttaaagacgaacctaactacacagaaaatttcattcaatcgatcatggaagctattccagagggttctaaaggtgccactcttgttgtcggtggtgatgggcgttactacaatgatgtcattcttcataagattgccgctatcggtgctgccaacggtattaaaaagttagttattggccagcatggtcttctgtctacgccagccgcttctcacatcatgagaacctacgaggaaaaatgtactggtggtattatcttaaccgcctcacataatccaggtggtccagaaaatgacatgggtattaagtataacttatccaatgggggtcctgctcctgaatccgtcacaaatgctatttgggagatttccaaaaagcttaccagctataagattatcaaagacttcccagaactagacttgggtacgataggcaagaacaagaaatacggtccattactcgttgacattatcgatattacaaaagattatgtcaacttcttgaaggaaatcttcgatttcgacttaatcaagaaattcatcgataatcaacgttctactaagaattggaagttactgtttgacagtatgaacggtgtaactggaccatacggtaaggctattttcgttgatgaatttggtttaccggcggatgaggttttacaaaactggcatccttctccggattttggtggtatgcatccagatccaaacttaacttatgccagttcgttagtgaaaagagtagatcgtgaaaagattgagtttggtgctgcatccgatggtgatggtgatagaaatatgatttacggttacggcccatctttcgtttctccaggtgactccgtcgcaattattgccgaatatgcagctgaaatcccatatttcgccaagcaaggtatatatggtctggcccgttcattccctacctcaggagccatagaccgtgttgccaaggctcatggtctaaactgttatgaggtcccaactggctggaaatttttctgtgctttgttcgacgctaaaaaattatctatttgtggtgaagaatcgtttggtactggttccaaccacgtaagggaaaaggacggtgtttgggccattatggcgtggttgaacatcttggccatttacaacaagcatcatccggagaacgaagcttctattaagacgatacagaatgaattttgggcaaagtacggccgtactttcttcactcgttatgattttgaaaaagttgaaacagaaaaagctaacaagattgtcgatcaattgagagcttatgttaccaaatcgggtgttgttaattccgccttcccagccgatgagtctcttaaggtcaccgattgtggtgatttttcatacacagatttggacggttctgtttctgaccatcaaggtttatatgtcaagctttccaatggtgcaagattcgttctaagattgtcaggtacaggttcttcaggtgctaccattagattgtacattgaaaaatactgcgatgataaatcacaataccaaaagacagctgaagaatacttgaagccaattattaactcggtcatcaagttcttgaactttaaacaagttttaggaactgaagaaccaacggttcgtacttaa

SEQ ID NO: 34 - DNA, 1629 bp, Saccharomyces cerevisiae
  source 1..1629: mol_type = other DNA
atggcaggagcaacatcaagtataattcgggaaaatgattttgaggacgagcttgcggagagtatgcagtcatataatagagaaactgcagacaaattggctttgactaggaccgagagtgtaaagccagaaccggagataaccgctccgcctcactcacgcttttcccgttctttcaagacagttttaatagctcagtgcgctttcactgggtttttctccacaatagcaggtgccatctactatccagttctgagcgttatagaaagaaaattcgatattgacgaggaattggtgaatgtcactgttgtagtatattttgtatttcagggtcttgcccccacattcatgggcgggtttgccgattcactgggcaggagaccggtggtgcttgtcgcaatcgtcatttattttggtgcctgcatcggtcttgcttgtgctcaaacgtatgctcagatcattgtgctaagatgtctacaagccgccggtatttcacccgtgattgcgattaacagcggaataatgggagatgttactactagagccgagcgcggcgggtacgttggatatgttgctggatttcaagttctaggttctgcgttcggagcccttatcggtgccggattatcatctagatggggatggagagccatcttttggttcttagcaattggatctggcatttgcttcctagcctcgtttttaattttgccagaaacaaagaggaacatatccgggaatggttctgtcaccccaaaatcatacttaaatagagctcccattcttgttttgccaacagtaagaaaatcattgcatttggataatccagattacgaaacattggaactgcccacgcaactaaatctactggcgcccttcaagattttgaaagcttatgaaatttgtatacttatgctagtcgctggattacaatttgccatgtatactacgcaccttacggcgttatccacagctttgagtaaacaatatcacttgactgttgcaaaggtaggattatgctaccttccctccggtatctgcacattatgtagtattgtcattgctggaagatatttgaattggaattacaggcgcagattaaaatattaccaaaattggttgggcaagaagagatcaaagcttttagaagaacacgacaacgaccttaatttggtgcaacgtatcatagaaaatgatcccaaatacacctttaatatcttcaaggcgaggttgcaacccgcatttgttaccttgcttttaagcagtagcggattttgtgcgtacggatggtgcattactgtcaaagcccctttagcggccgttctttgcatgagtggatttgcgtcgctgttttccaattgcattttgactttttcaacaactctaatagttgatctttttcccacgaagacatctacggctacgggatgtttaaaccttttcagatgcattctatctgctgtgtttatagctgcattgagtaaaatggtagaaaaaatgaaattcggaggtgtttttacatttttgggggctttaacctcctcgtcatcgatcctcttatttattctcttgagaaaaggtaaagaattagcctttaagaggaaaaagcaagaactgggagtaaattaa

SEQ ID NO: 35 - protein, 542 aa, Saccharomyces cerevisiae
MAGATSSIIRENDFEDELAESMQSYNRETADKLALTRTESVKPEPEITAPPHSRFSRSFKTVLIAQCAFTGFFSTIAGAIYYPVLSVIERKFDIDEELVNVTVVVYFVFQGLAPTFMGGFADSLGRRPVVLVAIVIYFGACIGLACAQTYAQIIVLRCLQAAGISPVIAINSGIMGDVTTRAERGGYVGYVAGFQVLGSAFGALIGAGLSSRWGWRAIFWFLAIGSGICFLASFLILPETKRNISGNGSVTPKSYLNRAPILVLPTVRKSLHLDNPDYETLELPTQLNLLAPFKILKAYEICILMLVAGLQFAMYTTHLTALSTALSKQYHLTVAKVGLCYLPSGICTLCSIVIAGRYLNWNYRRRLKYYQNWLGKKRSKLLEEHDNDLNLVQRIIENDPKYTFNIFKARLQPAFVTLLLSSSGFCAYGWCITVKAPLAAVLCMSGFASLFSNCILTFSTTLIVDLFPTKTSTATGCLNLFRCILSAVFIAALSKMVEKMKFGGVFTFLGALTSSSSILLFILLRKGKELAFKRKKQELGVN

SEQ ID NO: 36 - DNA, 1308 bp, Saccharomyces cerevisiae
  source 1..1308: mol_type = other DNA
atggactacaacaagagatcttcggtctcaaccgtgcctaatgcagctcccataagagtcggattcgtcggtctcaacgcagccaaaggatgggcaatcaagacacattaccccgccatactgcaactatcgtcacaatttcaaatcactgccttatacagtccaaaaattgagacttctattgccaccattcagcgtctaaaattgagtaatgccactgcttttcccactttagagtcatttgcatcatcttccactatagatatgatagtgatagctatccaagtggccagccattatgaagttgttatgcctctcttggaattctccaaaaataatccgaacctcaagtatcttttcgtagaatgggcccttgcatgttcactagatcaagccgaatccatttataaggctgctgctgaacgtggggttcaaaccatcatctctttacaaggtcgtaaatcaccatatattttgagagcaaaagaattaatatctcaaggctatatcggcgacattaattcgatcgagattgctggaaatggcggttggtacggctacgaaaggcctgttaaatcaccaaaatacatctatgaaatcgggaacggtgtagatctggtaaccacaacatttggtcacacaatcgatattttacaatacatgacaagttcgtacttttccaggataaatgcaatggttttcaataatattccagagcaagagctgatagatgagcgtggtaaccgattgggccagcgagtcccaaagacagtaccggatcatcttttattccaaggcacattgttaaatggcaatgttccagtgtcatgcagtttcaaaggtggcaaacctaccaaaaaatttaccaaaaatttggtcattgacattcacggtaccaagggagatttgaaacttgaaggcgatgccggcttcgcagaaatttcaaatctggtcctttactacagtggaactagagcaaacgacttcccgctagccaatggacaacaagctcctttagacccggggtatgatgcaggtaaagaaatcatggaagtatatcatttacgaaattataatgccattgtgggtaatattcatcgactgtatcaatctatctctgacttccacttcaatacaaagaaaattcctgaattaccctcacaatttgtaatgcaaggtttcgatttcgaaggctttcccaccttgatggatgctctgatattacacaggttaatcgagagcgtttataaaagtaacatgatgggctccacattaaacgttagcaatatctcgcattatagtttataa

SEQ ID NO: 37 - protein, 435 aa, Saccharomyces cerevisiae
MDYNKRSSVSTVPNAAPIRVGFVGLNAAKGWAIKTHYPAILQLSSQFQITALYSPKIETSIATIQRLKLSNATAFPTLESFASSSTIDMIVIAIQVASHYEVVMPLLEFSKNNPNLKYLFVEWALACSLDQAESIYKAAAERGVQTIISLQGRKSPYILRAKELISQGYIGDINSIEIAGNGGWYGYERPVKSPKYIYEIGNGVDLVTTTFGHTIDILQYMTSSYFSRINAMVFNNIPEQELIDERGNRLGQRVPKTVPDHLLFQGTLLNGNVPVSCSFKGGKPTKKFTKNLVIDIHGTKGDLKLEGDAGFAEISNLVLYYSGTRANDFPLANGQQAPLDPGYDAGKEIMEVYHLRNYNAIVGNIHRLYQSISDFHFNTKKIPELPSQFVMQGFDFEGFPTLMDALILHRLIESVYKSNMMGSTLNVSNISHYSL

SEQ ID NO: 38 - DNA, 1587 bp, Saccharomyces cerevisiae
  source 1..1587: mol_type = other DNA
atgactaaatctcattcagaagaagtgattgtacctgagttcaattctagcgcaaaggaattaccaagaccattggccgaaaagtgcccgagcataattaagaaatttataagcgcttatgatgctaaaccggattttgttgctagatcgcctggtagagtcaatctaattggtgaacatattgattattgtgacttctcggttttacctttagctattgattttgatatgctttgcgccgtcaaagttttgaacgagaaaaatccatccattaccttaataaatgctgatcccaaatttgctcaaaggaagttcgatttgccgttggacggttcttatgtcacaattgatccttctgtgtcggactggtctaattactttaaatgtggtctccatgttgctcactcttttctaaagaaacttgcaccggaaaggtttgccagtgctcctctggccgggctgcaagtcttctgtgagggtgatgtaccaactggcagtggattgtcttcttcggccgcattcatttgtgccgttgctttagctgttgttaaagcgaatatgggccctggttatcatatgtccaagcaaaatttaatgcgtattacggtcgttgcagaacattatgttggtgttaacaatggcggtatggatcaggctgcctctgtttgcggtgaggaagatcatgctctatacgttgagttcaaaccgcagttgaaggctactccgtttaaatttccgcaattaaaaaaccatgaaattagctttgttattgcgaacacccttgttgtatctaacaagtttgaaaccgccccaaccaactataatttaagagtggtagaagtcactacagctgcaaatgttttagctgccacgtacggtgttgttttactttctggaaaagaaggatcgagcacgaataaaggtaatctaagagatttcatgaacgtttattatgccagatatcacaacatttccacaccctggaacggcgatattgaatccggcatcgaacggttaacaaagatgctagtactagttgaagagtctctcgccaataagaaacagggctttagtgttgacgatgtcgcacaatccttgaattgttctcgcgaagaattcacaagagactacttaacaacatctccagtgagatttcaagtcttaaagctatatcagagggctaagcatgtgtattctgaatctttaagagtcttgaaggctgtgaaattaatgactacagcgagctttactgccgacgaagactttttcaagcaatttggtgccttgatgaacgagtctcaagcttcttgcgataaactttacgaatgttcttgtccagagattgacaaaatttgttccattgctttgtcaaatggatcatatggttcccgtttgaccggagctggctggggtggttgtactgttcacttggttccagggggcccaaatggcaacatagaaaaggtaaaagaagcccttgccaatgagttctacaaggtcaagtaccctaagatcactgatgctgagctagaaaatgctatcatcgtctctaaaccagcattgggcagctgtctatatgaattataa

SEQ ID NO: 39 - protein, 528 aa, Saccharomyces cerevisiae
MTKSHSEEVIVPEFNSSAKELPRPLAEKCPSIIKKFISAYDAKPDFVARSPGRVNLIGEHIDYCDFSVLPLAIDFDMLCAVKVLNEKNPSITLINADPKFAQRKFDLPLDGSYVTIDPSVSDWSNYFKCGLHVAHSFLKKLAPERFASAPLAGLQVFCEGDVPTGSGLSSSAAFICAVALAVVKANMGPGYHMSKQNLMRITVVAEHYVGVNNGGMDQAASVCGEEDHALYVEFKPQLKATPFKFPQLKNHEISFVIANTLVVSNKFETAPTNYNLRVVEVTTAANVLAATYGVVLLSGKEGSSTNKGNLRDFMNVYYARYHNISTPWNGDIESGIERLTKMLVLVEESLANKKQGFSVDDVAQSLNCSREEFTRDYLTTSPVRFQVLKLYQRAKHVYSESLRVLKAVKLMTTASFTADEDFFKQFGALMNESQASCDKLYECSCPEIDKICSIALSNGSYGSRLTGAGWGGCTVHLVPGGPNGNIEKVKEALANEFYKVKYPKITDAELENAIIVSKPALGSCLYEL

SEQ ID NO: 40 - DNA, 2100 bp, Saccharomyces cerevisiae
  source 1..2100: mol_type = other DNA
atgacagctcagttacaaagtgaaagtacttctaaaattgttttggttacaggtggtgctggatacattggttcacacactgtggtagagctaattgagaatggatatgactgtgttgttgctgataacctgtcgaattcaacttatgattctgtagccaggttagaggtcttgaccaagcatcacattcccttctatgaggttgatttgtgtgaccgaaaaggtctggaaaaggttttcaaagaatataaaattgattcggtaattcactttgctggtttaaaggctgtaggtgaatctacacaaatcccgctgagatactatcacaataacattttgggaactgtcgttttattagagttaatgcaacaatacaacgtttccaaatttgttttttcatcttctgctactgtctatggtgatgctacgagattcccaaatatgattcctatcccagaagaatgtcccttagggcctactaatccgtatggtcatacgaaatacgccattgagaatatcttgaatgatctttacaatagcgacaaaaaaagttggaagtttgctatcttgcgttattttaacccaattggcgcacatccctctggattaatcggagaagatccgctaggtataccaaacaatttgttgccatatatggctcaagtagctgttggtaggcgcgagaagctttacatcttcggagacgattatgattccagagatggtaccccgatcagggattatatccacgtagttgatctagcaaaaggtcatattgcagccctgcaatacctagaggcctacaatgaaaatgaaggtttgtgtcgtgagtggaacttgggttccggtaaaggttctacagtttttgaagtttatcatgcattctgcaaagcttctggtattgatcttccatacaaagttacgggcagaagagcaggtgatgttttgaacttgacggctaaaccagatagggccaaacgcgaactgaaatggcagaccgagttgcaggttgaagactcctgcaaggatttatggaaatggactactgagaatccttttggttaccagttaaggggtgtcgaggccagattttccgctgaagatatgcgttatgacgcaagatttgtgactattggtgccggcaccagatttcaagccacgtttgccaatttgggcgccagcattgttgacctgaaagtgaacggacaatcagttgttcttggctatgaaaatgaggaagggtatttgaatcctgatagtgcttatataggcgccacgatcggcaggtatgctaatcgtatttcgaagggtaagtttagtttatgcaacaaagactatcagttaaccgttaataacggcgttaatgcgaatcatagtagtatcggttctttccacagaaaaagatttttgggacccatcattcaaaatccttcaaaggatgtttttaccgccgagtacatgctgatagataatgagaaggacaccgaatttccaggtgatctattggtaaccatacagtatactgtgaacgttgcccaaaaaagtttggaaatggtatataaaggtaaattgactgctggtgaagcgacgccaataaatttaacaaatcatagttatttcaatctgaacaagccatatggagacactattgagggtacggagattatggtgcgttcaaaaaaatctgttgatgtcgacaaaaacatgattcctacgggtaatatcgtcgatagagaaattgctacctttaactctacaaagccaacggtcttaggccccaaaaatccccagtttgattgttgttttgtggtggatgaaaatgctaagccaagtcaaatcaatactctaaacaatgaattgacgcttattgtcaaggcttttcatcccgattccaatattacattagaagttttaagtacagagccaacttatcaattttataccggtgatttcttgtctgctggttacgaagcaagacaaggttttgcaattgagcctggtagatacattgatgctatcaatcaagagaactggaaagattgtgtaaccttgaaaaacggtgaaacttacgggtccaagattgtctacagattttcctga

SEQ ID NO: 41 - protein, 699 aa, Saccharomyces cerevisiae
MTAQLQSESTSKIVLVTGGAGYIGSHTVVELIENGYDCVVADNLSNSTYDSVARLEVLTKHHIPFYEVDLCDRKGLEKVFKEYKIDSVIHFAGLKAVGESTQIPLRYYHNNILGTVVLLELMQQYNVSKFVFSSSATVYGDATRFPNMIPIPEECPLGPTNPYGHTKYAIENILNDLYNSDKKSWKFAILRYFNPIGAHPSGLIGEDPLGIPNNLLPYMAQVAVGRREKLYIFGDDYDSRDGTPIRDYIHVVDLAKGHIAALQYLEAYNENEGLCREWNLGSGKGSTVFEVYHAFCKASGIDLPYKVTGRRAGDVLNLTAKPDRAKRELKWQTELQVEDSCKDLWKWTTENPFGYQLRGVEARFSAEDMRYDARFVTIGAGTRFQATFANLGASIVDLKVNGQSVVLGYENEEGYLNPDSAYIGATIGRYANRISKGKFSLCNKDYQLTVNNGVNANHSSIGSFHRKRFLGPIIQNPSKDVFTAEYMLIDNEKDTEFPGDLLVTIQYTVNVAQKSLEMVYKGKLTAGEATPINLTNHSYFNLNKPYGDTIEGTEIMVRSKKSVDVDKNMIPTGNIVDREIATFNSTKPTVLGPKNPQFDCCFVVDENAKPSQINTLNNELTLIVKAFHPDSNITLEVLSTEPTYQFYTGDFLSAGYEARQGFAIEPGRYIDAINQENWKDCVTLKNGETYGSKIVYRFS

SEQ ID NO: 42 - DNA, 1476 bp, Chenopodium quinoa
  source 1..1476: mol_type = other DNA
atggaatcttctactaaggaacaagaacaacaacacatcatcttgttgccattcttggctcaaggtcacttgagaccattcttgcacttggctcacagattgttgtctttgactccattcaacttgtctttgttgactactccattgaacgctgctaacttgagaagacaatctgacaacttcaacatcaacttgaacatcatcgaattgccattcacttctactgaccacggtttgccaccaaacactgaaaacactgacaagttgtctttgacttctatcatcaagttgttccacgcttctacttctttggaaccacacgttagagactacttgactagacaccacttgcacgacccaccagtttgtatcatcttcgacgttttcttgggttgggctgacaacgttgctagatctgttggttctactggtatctgtttcaacactggtggtgcttacggtgttggtgcttacgtttctatctggtctaacttgccacacagaaacgttggtgacgacgaagaattttctttggctggtttcccagaagacagaaagttgagaagaaaccaattgcacagattcttgagattcgctgacggttctgacgaatggtcaagattcttccaaccacaaatcaagtcttctttgaactgttctggttggttgtgtaactctatcgaagaaatcgaaccattgggtttccaagttttcagaaacttcactaagtctccaatctggggtatcggtccattgatcatgacttcttctaagaaggacgacgacgaaaaggaagaagcttgtttgagatggttgaaccaattcgaaaacgactctgttttgtacatctgtttcggttctcaaaacactgttactccaatccaaatgatggaattggctaagggtttggaagaatctaagatcccattcttgtgggttatcagaccaccattcggtttcgacttcaacggtgaatttaagccagaatggttgccagaaaagttcgaagaaagaatgatggaaaagaagcaaggtatgttggttagagactgggttccacaattggacatcttgagacacgaagctactggtggtttcttgtctcactgtggttggaactctgttttggaaggtttgagagaaggtgttccaatcttgggttggccattggctgctgaacaagcttacaactctaagatgatggttgaagaaatgggtgttgctgttgaattgactagaggtttggaaggtgaagttaagaaggaatgggttaagtctgttgttgaaatggttttggacagaaaggaaggttcttgtggttgggaaatgaagaagaaggctgttgaaatcggtgaaaagttgaaggacgcttggaagatcgaaggtgactacaagggttcttctgttaaggctatggacaacttcgttgaatttatcgtttcttgtagaggtaagaagtctaaggaagacgcttaa

SEQ ID NO: 43 - protein, 491 aa, Chenopodium quinoa
MESSTKEQEQQHIILLPFLAQGHLRPFLHLAHRLLSLTPFNLSLLTTPLNAANLRRQSDNFNINLNIIELPFTSTDHGLPPNTENTDKLSLTSIIKLFHASTSLEPHVRDYLTRHHLHDPPVCIIFDVFLGWADNVARSVGSTGICFNTGGAYGVGAYVSIWSNLPHRNVGDDEEFSLAGFPEDRKLRRNQLHRFLRFADGSDEWSRFFQPQIKSSLNCSGWLCNSIEEIEPLGFQVFRNFTKSPIWGIGPLIMTSSKKDDDEKEEACLRWLNQFENDSVLYICFGSQNTVTPIQMMELAKGLEESKIPFLWVIRPPFGFDFNGEFKPEWLPEKFEERMMEKKQGMLVRDWVPQLDILRHEATGGFLSHCGWNSVLEGLREGVPILGWPLAAEQAYNSKMMVEEMGVAVELTRGLEGEVKKEWVKSVVEMVLDRKEGSCGWEMKKKAVEIGEKLKDAWKIEGDYKGSSVKAMDNFVEFIVSCRGKKSKEDA

SEQ ID NO: 44 - DNA, 665 bp, synthetic construct
  source 1..665: mol_type = other DNA
tttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaac

(19) United States
(12) Patent Application Publication
(10) Pub. No.: US 2026/0357069 A1
(43) Pub. Date: Nov. 12, 2026

(54) BETACYANIN PIGMENT COMPOSITIONS

(71) Applicant: PHYTOLON LTD., Yokne’am Illit (IL)

(72) Inventors: Halim JUBRAN, Haifa (IL); Tal ZELTZER, Kiryat Tivon (IL); Wissam MANSOUR, Rama Village (IL); Evyatar STEINER, Beit El (IL); Doron LEIZER, Nesher (IL)

(21) Appl. No.: 19/707,747

(22) Filed: Jun. 15, 2026

Related U.S. Application Data
(63) Continuation of application No. PCT/IL2024/051179, filed on Dec. 12, 2024.
(60) Provisional application No. 63/609,898, filed on Dec. 14, 2023.
(60) Provisional application No. 63/690,887, filed on Sep. 5, 2024.

(51) Int. Cl. C09B 61/00; C12N 1/16; C12N 9/02; C12N 9/10; C12N 15/52; C12R 1/645

(52) U.S. Cl. CPC C09B 61/00; C12N 1/16; C12N 9/004; C12N 9/0042; C12N 9/0069; C12N 9/0071; C12N 9/1051; C12N 15/52; C12Y 106/02002; C12Y 106/02004; C12Y 113/11029; C12R 2001/645

(57) ABSTRACT

Pigment compositions comprising betalain pigment molecules are provided. Compositions comprising betanin and betanidin are also provided as are compositions comprising less than 5% sugars by weight. Cells comprising at least one copy of an exogenous tyrosine hydroxylase gene, at least one copy of an exogenous L-DOPA oxidase gene, at least one copy of an exogenous DOPA 4,5-dioxygenase (DOD) gene, at least one copy of an exogenous NADPH cytochrome P450 reductase gene, and at least one copy of a cyclo-DOPA-5-O-glucosyltransferase gene integrated into the genome of the cell are also provided.

### CROSS REFERENCE TO RELATED APPLICATIONS

This application is a Bypass Continuation of PCT Patent Application No. PCT/IL2024/051179 having International filing date of Dec. 12, 2024, which claims the benefit of priority of U.S. Provisional Patent Application No. 63/609,898, filed Dec. 14, 2023, and U.S. Provisional Patent Application No. 63/690,887, filed Sep. 5, 2024, the contents of which are all incorporated herein by reference in their entirety.

### REFERENCE TO AN ELECTRONIC SEQUENCE LISTING

The contents of the electronic sequence listing (PHYT-P-002-PCT.xml; Size: 82,588 bytes; and Date of Creation: Dec. 10, 2024) are herein incorporated by reference in its entirety.

### FIELD OF INVENTION

The present invention is in the field of pigment compositions.

### BACKGROUND OF THE INVENTION

Betalains are tyrosine-derived, red-violet and yellow plant pigments found in only one group of angiosperms, the Caryophyllales order, in which they occur in a mutually exclusive fashion with the chemically distinct and widespread anthocyanin pigments. The betalain class contains a wide array of compounds, which are generally classified into two groups; the red-violet betacyanins, and the yellow betaxanthins.

Due to their high water solubility, pH stability and antioxidative properties, betalains may be used as natural food colorants and dietary supplements. While potential edible plant sources of anthocyanins are numerous, betalains are found in very few edible plants, with red beet being the only major source for betalain extraction in commercial use today. Despite its high betacyanin content, red beet extract has several drawbacks as a source of food colorants; it mainly produces betanin and thus has limited color variability, and it carries adverse earthy flavors due to the occurrence of geosmin and various pyrazines. These compounds are considered undesirable due to their distinctive taste and odor, which can impart an unpleasant earthy flavor to food products, potentially compromising the intended taste profile. Red beet extract further contains a high carbohydrate and specifically sugar (mainly sucrose, glucose and fructose) content, and holds the risk of carry-over of soil-borne microbes, nitrates, and pesticides. Nitrates in foods are undesirable because they can undergo chemical reactions that lead to formation of nitrosamines, compounds that have been associated with potential health risks, including their classification as possible carcinogens.

New methods of producing betalains in cultured microbial cells are provided in International Patent Publications WO2017/122189 and WO2022/253815 and Tinggaard Thomsen, “Beet red food colourant can be produced more sustainably with engineered Yarrowia lipolytica”, Nat Microbiol., 2023 December; 8(12):2290-2303. A new superior formulation of betacyanin pigment is greatly needed.

### SUMMARY OF THE INVENTION

The present invention provides pigment compositions comprising betalain pigment molecules. Compositions comprising betanin and betanidin are provided. Compositions comprising betalain pigment molecules and less than 5% sugars by weight are also provided. Cells comprising at least one copy of an exogenous tyrosine hydroxylase gene, at least one copy of an exogenous L-DOPA oxidase gene, at least one copy of an exogenous DOPA 4,5-dioxygenase (DOD) gene, at least one copy of an exogenous NADPH cytochrome P450 reductase gene, and at least one copy of a cyclo-DOPA-5-O-glucosyltransferase gene integrated into the genome of the cell are also provided.

According to a first aspect, there is provided a pigment composition comprising betalain pigment molecules, wherein the betalain pigment molecules comprise betanin and betanidin.

According to another aspect, there is provided a pigment composition comprising betalain pigment molecules, wherein the composition comprises less than 5% mono- and disaccharides by weight.

According to some embodiments, the betalain pigment molecules comprise a betacyanin.

According to some embodiments, the betalain pigment molecules comprise betanin and betanidin.

According to some embodiments, the betanidin is at least 0.5% of the betalain pigment molecules.

According to some embodiments, the betanidin is at least 10% of the betalain pigment molecules.

According to some embodiments, the betanidin is between 1% and 90% of the betalain pigment molecules.

According to some embodiments, the betanidin is between 10% and 75% of the betalain pigment molecules.

According to some embodiments, the composition comprises less than 5% sugars by weight.

According to some embodiments, less than 5% sugars by weight is less than 1% sugars by weight.

According to some embodiments, the composition comprises less than 0.1 μg/kg geosmin, less than 0.01 g/L nitrates or both.

According to some embodiments, the betalain pigment molecules further comprise iso-betanidin.

According to some embodiments, a ratio of betanidin to iso-betanidin is at least 5:1.

According to some embodiments, the betanin is at least 10% of the betalain pigment molecules.

According to some embodiments, the betanin is at least 50% of the betalain pigment molecules.

According to some embodiments, the betanin is between 10% and 90% of the betalain pigment molecules. According to some embodiments, the betanin is between 10% and 99% of the betalain pigment molecules.

According to some embodiments, the betanin is between 50% and 90% of the betalain pigment molecules. According to some embodiments, the betanin is between 50% and 99% of the betalain pigment molecules.

According to some embodiments, the betalain pigment molecules comprise betanin and iso-betanin and wherein a ratio of betanin to iso-betanin in the pigment composition is more than 7:1.

According to some embodiments, the composition is a liquid composition or a powder.

According to some embodiments, the composition is a powder.

According to some embodiments, the pigment composition comprises less than 10% dietary carbohydrates by weight.

According to some embodiments, the pigment composition comprises less than 10% betaxanthins.

According to some embodiments, the pigment composition comprises less than 10% neobetanin.

According to some embodiments, the pigment composition comprises at least 500 mg magnesium per liter of composition or at least 1000 mg magnesium per kilogram of composition.

According to some embodiments, the pigment composition comprises less than 50 mg sodium per liter of composition.

According to some embodiments, the pigment composition is a liquid and comprises greater than 1.5 grams betalain pigment molecules per liter of composition.

According to some embodiments, the pigment is a powder and comprises greater than 1.5 grams betalain pigment molecules per kilogram of composition.

According to some embodiments, the pigment composition is essentially devoid of intact cells.

According to another aspect, there is provided a cell comprising at least one copy of an exogenous tyrosine hydroxylase gene, at least one copy of an exogenous L-DOPA oxidase gene, at least one copy of an exogenous DOPA 4,5-dioxygenase (DOD) gene, at least one copy of an exogenous NADPH cytochrome P450 reductase gene, and at least one copy of a cyclo-DOPA-5-O-glucosyltransferase gene integrated into the genome of the cell.

According to some embodiments, the tyrosine hydroxylase gene and the L-DOPA oxidase gene are the same gene.

According to some embodiments, the tyrosine hydroxylase gene and the L-DOPA oxidase gene are CYP76AD1.

According to some embodiments, the DOD is Mirabilis jalapa DOD (MjDOD).

According to some embodiments, the reductase is NADPH-cytochrome P450 reductase 2 (ATR2) or NADPH-cytochrome P450 reductase 1 (ATR1).

According to some embodiments, the cyclo-DOPA-5-O-glucosyltransferase is a Mirabilis jalapa cyclo-DOPA-5-O-glucosyltransferase (MjcDOPA5GT), a Chenopodium quinoa cyclo-DOPA-5-O-glucosyltransferase (CqcDOPA5GT) or both.

According to some embodiments, the cell comprises at least two copies of the exogenous tyrosine hydroxylase gene, at least two copies of the exogenous L-DOPA oxidase gene, at least two copies of the exogenous reductase gene and at least two copies of the exogenous glycosyl transferase gene integrated in the genome of the cell.

According to some embodiments, the cell comprises 3 copies of a CYP76AD1 gene, 1 copy of a DOD gene, 3 copies of an NADPH cytochrome P450 reductase gene and 3 copies of a cyclo-DOPA-5-O-glucosyltransferase gene integrated into the genome of the cell.

According to some embodiments, the cell comprises at least two copies of the exogenous DOD gene.

According to some embodiments, the cell comprises 4 copies of a CYP76AD1 gene, 3 copies of a MjDOD gene, 2 copies of an ATR2 gene and at least 2 copies of a Mj5GT gene integrated into the genome of the cell. According to some embodiments, the cell comprises 5 copies of a CYP76AD1 gene, 2 or 3 copies of a DOD gene, 4 copies of an NADPH cytochrome P450 reductase gene and 3 copies of a cyclo-DOPA-5-O-glucosyltransferase gene integrated into the genome of the cell.

According to some embodiments, the cell comprises at least 1 copy of an ATR1 gene integrated into the genome of the cell.

According to some embodiments, the cell further comprises at least one copy of an exogenous Cytochrome b5 reductase (Cyb5) gene integrated into the genome of the yeast cell.

According to some embodiments, the cell further comprises at least one copy of an exogenous betanidin-5-O-glucosyltransferase gene integrated into the genome of the yeast cell.

According to some embodiments, the betanidin-5-O-glucosyltransferase is a Dorotheanthus bellidiformis betanidin-5-O-glucosyltransferase (Db5GT).

According to some embodiments, the cell further comprises at least one copy of an exogenous gene integrated into the genome of the cell, at least one knockout of an endogenous gene in the cell or both that result in increased production of UDP-glucose.

According to some embodiments, the exogenous gene is selected from: UGP1, PGM2, URA6 and YNK1; the endogenous gene is selected from exg1 and ynd1; or both.

According to some embodiments, the cell comprises at least one exogenous copy of each of PGM2, URA6 and YNK1 integrated into the genome of the cell and knockout of endogenous exg1 and endogenous ynd1 in the cell.

According to some embodiments, the cell further comprises at least one copy of an exogenous plasma membrane transporter gene integrated into the genome of the cell.

According to some embodiments, the plasma membrane transporter is a yeast plasma membrane transporter. According to some embodiments, the yeast plasma membrane transporter is QDR2.

According to some embodiments, the cell further comprises knockdown of at least one endogenous gene of the galactose metabolism pathway in the cell.

According to some embodiments, the cell comprises knockdown of endogenous gal80, gal1 and gal10.

According to some embodiments, the cell is a plant cell, a bacterial cell or a fungal cell.

According to some embodiments, the fungal cell is a unicellular fungal cell.

According to some embodiments, the unicellular fungal cell is a yeast cell.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Schematic representation of fermentation-based betacyanin manufacturing process. (a) The fermentation process includes medium preparation and sterilization, seed fermentations for main fermenter inoculation, batch phase in main fermenter (optional) and a carbohydrate (glucose, sucrose or other carbon source) feeding stage (fed-batch). (b) Broth harvesting includes a yeast clearance step like centrifugation or microfiltration that separates the broth (containing the product) from the biomass. (c) removal of proteins and salts by ultrafiltration and/or nanofiltration and final evaporation steps are optional.

FIG. 2: Table of characteristics of betalain pigment compositions of the invention and previously known compositions.

FIG. 3: Images of the color of betalain pigment compositions of the invention and previously known compositions.

FIG. 4: Color Point measurement values as compared to Fresh Beetroot Juice extract.

FIG. 5: Table of genomic integrations and characteristics of high betanin yeast strains of the invention.

FIG. 6: Table of genomic integrations and characteristics of optimized betanin yeast strains of the invention.

FIG. 7: Images of the color of high-betanin betalain pigment compositions of the invention. Color Point measurement values are provided.

### DETAILED DESCRIPTION OF THE INVENTION

The present invention, in some embodiments, provides pigment compositions comprising betalain pigment molecules.

The invention is based, at least in part, on the surprising production from unique engineered yeast of heretofore unattainable color compositions. The color pigments betanidin and betanin are different to the human eye and thus by varying the percentage of each molecule in a pigment composition different hues can be produced. In natural betacyanin compositions (e.g., beetroot) only betanin is present. In previous productions in yeast only betanidin was produced. This greatly limits the color options. In the instant application it is described how the production of betanin in yeast can be greatly increased through genetic modification of the yeast. The result is that compositions with essentially any ratio of betanin to betanidin (or vice-versa) can be produced. Even compositions from yeast with 99% betanin, essentially equivalent in betacyanin composition to the natural beet extract, could be produced, but without the high sugar content inherent to beet extracts and without contaminants such as geosmin, nitrates and sodium.

By a first aspect, there is provided a composition comprising betalain pigment molecules.

In some embodiments, the composition is a pigment composition. In some embodiments, the composition is an artificial composition. In some embodiments, the composition does not occur in nature. In some embodiments, the composition is a mixture. In some embodiments, the ratio of betalains in the mixture does not occur in nature. In some embodiments, the composition is non-natural. In some embodiments, the pigment is a betalain pigment. In some embodiments, the pigment is a red pigment. In some embodiments, the pigment is a purple pigment. In some embodiments, the pigment is within the red spectrum. In some embodiments, the pigment is within the red-violet spectrum. In some embodiments, the red pigment reflects light with a wavelength between 620 and 750 nanometers. In some embodiments, the red pigment reflects light with a wavelength between 625 and 740 nanometers. In some embodiments, the red pigment reflects light with a wavelength between 625 and 750 nanometers. In some embodiments, the red pigment reflects light with a wavelength between 620 and 700 nanometers. In some embodiments, the red pigment comprises a lambda max (λₘₐₓ) of about 520-550 nanometers. In some embodiments, the red pigment comprises a lambda max (λₘₐₓ) of about 525-545 nanometers. In some embodiments, the red pigment comprises a lambda max (λₘₐₓ) of about 530-540 nanometers. In some embodiments, the red pigment comprises a lambda max (λₘₐₓ) of about 530-535 nanometers. In some embodiments, the red pigment comprises a lambda max (λₘₐₓ) of about 530-545 nanometers. In some embodiments, the red pigment comprises a lambda max (λₘₐₓ) of about 530-550 nanometers. In some embodiments, the red pigment comprises a lambda max (λₘₐₓ) of about 535-540 nanometers. In some embodiments, the red pigment comprises a lambda max (λₘₐₓ) of about 535-545 nanometers. In some embodiments, the red pigment comprises a lambda max (λₘₐₓ) of about 535-550 nanometers. It will be understood by a skilled artisan that the exact color of the composition is determined by the abundance of each betalain pigment and that the final color can be varied by varying the relative amounts of the various betalains in the composition.

In some embodiments, the composition is an edible composition. In some embodiments, the composition is organic. In some embodiments, the composition is fit for human consumption. In some embodiments, the composition is a dye. In some embodiments, the composition is a food coloring. In some embodiments, the composition is an additive. In some embodiments, the composition is a coloring composition. In some embodiments, the composition is a cookable composition. In some embodiments, the composition is thermostable. In some embodiments, the composition is a liquid composition. In some embodiments, the composition is aqueous. In some embodiments, the composition is a solid. In some embodiments, the solid is a powder. In some embodiments, the composition is a crystal. In some embodiments, the composition is a non-natural composition. In some embodiments, the composition comprises betalain pigment molecule combinations and ratios not found in nature. In some embodiments, the composition comprises a color not found in nature. Because the color of the pigment composition of the invention is not found in nature the composition of the invention cannot be considered a product of nature.

Betalains are water-soluble pigments derived from tyrosine present in vacuoles of plants of the order Caryophyllales and in mushrooms of the genera Amanita, Hygrocybe and Hygrophorus. Within the plant kingdom, betalains are found in only one group of angiosperms, the Caryophyllales. In this order, betalains and anthocyanins occur in a mutually exclusive fashion, i.e., no plant species produces both types of pigments. One of the most prominent features of the Caryophyllales order is its dominance in arid and semi-arid regions, and habitation of saline and alkaline soils. While some families within the Caryophyllales are distributed worldwide in a variety of habitats, members of several families are particularly adapted to arid or saline regions, including the Aizoaceae (ice-plant family), Portulacaceae (purslane family) and most notably, the Cactaceae (cactus family).

Betalains are divided into two classes of pigment molecules: red colored betacyanins and yellow colored betaxanthins. In some embodiments, the betalain pigment molecule is a betacyanin. In some embodiments, the betalain pigment molecule is a betacyanin pigment molecule. In some embodiments, the betalain pigment molecules comprise a betacyanin. In some embodiments, the betalain pigment molecules comprise a betaxanthin. In some embodiments, the betalain pigment molecules comprise betacyanins and betaxanthins. In some embodiments, the betalain pigment molecules are devoid of a betaxanthin.

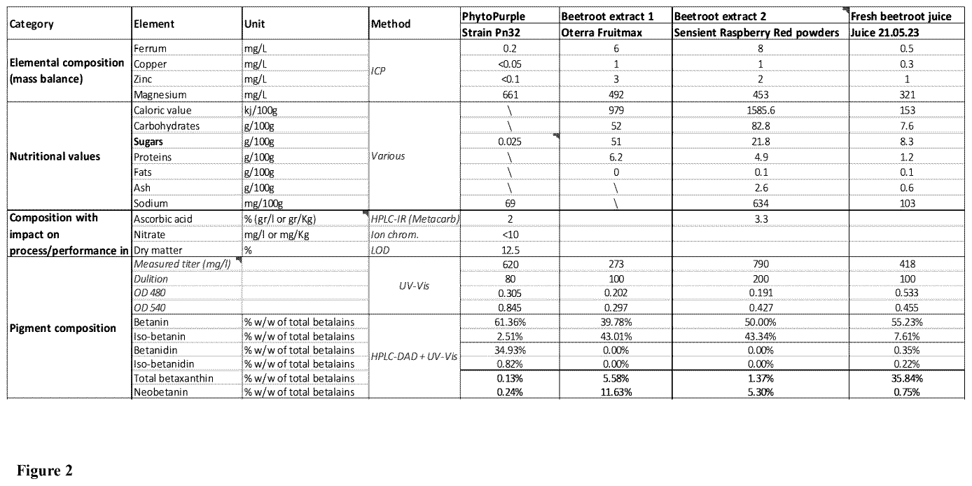

In some embodiments, the betalain pigment molecules comprise betanin and betanidin. In some embodiments, the betalain pigment molecule comprises betacyanins and betacyanidins. In some embodiments, the betacyanins are selected from betanin and betanidin. In some embodiments, the betacyanins comprise stereoisomers of the betacyanins. In some embodiments, the betacyanins are selected from betanin, iso-betanin, betanidin and iso-betanidin. In some embodiments, the stereoisomer of betanin is iso-betanin. In some embodiments, the stereoisomer of betanidin is iso-betanidin. In some embodiments, the composition comprises betanin and betanidin. Betanin is the betacyanin found in red beets. Betanin is a glucoside and hydrolyzes into glucose and betanidin. As demonstrated hereinbelow, natural beet extracts and juice do not contain measurable levels of betanidin (FIG. 2). Further, previous examples of betacyanins produced by fermentation did not contain measurable levels of betanin (International Patent Publication WO2017/122189, Table 2). The composition of the invention is thus the first to contain both measurable (and visible) levels of betanin and measurable (and visible) levels of betanidin. In some embodiments, the composition comprises betanin and iso-betanin. In some embodiments, the composition comprises betanidin and iso-betanidin. In some embodiments, the composition comprises betanin, iso-betanin, betanidin and iso-betanidin.

In some embodiments, at least 0.01, 0.05, 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45 or 50% of the betalain pigment molecules is betanidin. Each possibility represents a separate embodiment of the invention. In some embodiments, at least 0.5% of said betalain pigment molecules is betanidin. In some embodiments, at least 1% of said betalain pigment molecules is betanidin. In some embodiments, at least 5% of said betalain pigment molecules is betanidin. In some embodiments, at least 7.5% of said betalain pigment molecules is betanidin. In some embodiments, at least 9.5% of said betalain pigment molecules is betanidin. In some embodiments, at least 10% of said betalain pigment molecules is betanidin. In some embodiments, at least 15% of said betalain pigment molecules is betanidin. In some embodiments, at least 35% of said betalain pigment molecules is betanidin. It will be understood that the statement that at least 10% of the betalain pigment molecules is betanidin is equivalent to the statement that betanidin is at least 10% of the betalain pigment molecules. The same is true for similar statements with other pigment molecules and/or percentages. It will be understood, that except when discussing the betanidin to iso-betanidin ratio, the general term betanidin is used so as to encompass both betanidin and iso-betanidin.

In some embodiments, at least 0.01, 0.05, 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45 or 50% of the betalain pigment molecules is betanidin and/or iso-betanidin. Each possibility represents a separate embodiment of the invention. In some embodiments, at least 0.5% of said betalain pigment molecules is betanidin and/or iso-betanidin. In some embodiments, at least 1% of said betalain pigment molecules is betanidin and/or iso-betanidin. In some embodiments, at least 5% of said betalain pigment molecules is betanidin and/or iso-betanidin. In some embodiments, at least 7.5% of said betalain pigment molecules is betanidin and/or iso-betanidin. In some embodiments, at least 9.5% of said betalain pigment molecules is betanidin and/or iso-betanidin. In some embodiments, at least 10% of said betalain pigment molecules is betanidin and/or iso-betanidin. In some embodiments, at least 15% of said betalain pigment molecules is betanidin and/or iso-betanidin. In some embodiments, at least 35% of said betalain pigment molecules is betanidin and/or iso-betanidin.

In some embodiments, at least 50, 55, 60, 65, 70, 71, 72, 73, 74, 75, 76, 77, 78, 79, 80, 85, 90, 92, 95, 97, 98 or 99% of the betalain pigment molecules is betanin. Each possibility represents a separate embodiment of the invention. In some embodiments, at least 50% of said betalain pigment molecules is betanin. In some embodiments, at least 55% of said betalain pigment molecules is betanin. In some embodiments, at least 60% of said betalain pigment molecules is betanin. In some embodiments, at least 70% of said betalain pigment molecules is betanin. In some embodiments, at least 72% of said betalain pigment molecules is betanin. In some embodiments, at least 90% of said betalain pigment molecules is betanin. In some embodiments, at least 95% of said betalain pigment molecules is betanin. In some embodiments, at least 98% of said betalain pigment molecules is betanin. It will be understood, that except when discussing the betanin to iso-betanin ratio and relationship, the general term betanin is used so as to encompass both betanin and iso-betanin. In some embodiments, betanin is betanin and iso-betanin.

In some embodiments, at least 50, 55, 60, 65, 70, 71, 72, 73, 74, 75, 76, 77, 78, 79, 80, 85, or 90% of the betalain pigment molecules is betanin and/or iso-betanin. Each possibility represents a separate embodiment of the invention. In some embodiments, at least 50% of said betalain pigment molecules is betanin and/or iso-betanin. In some embodiments, at least 55% of said betalain pigment molecules is betanin and/or iso-betanin. In some embodiments, at least 60% of said betalain pigment molecules is betanin and/or iso-betanin. In some embodiments, at least 70% of said betalain pigment molecules is betanin and/or iso-betanin. In some embodiments, at least 72% of said betalain pigment molecules is betanin and/or iso-betanin.

In some embodiments, at most 1, 2, 5, 7, 10, 12, 15, 17, 20, 25, 30, 35, 40, 45, or 50% of the betalain pigment molecules is betanidin. Each possibility represents a separate embodiment of the invention. In some embodiments, at most 40% of the betalain pigment molecules is betanidin. In some embodiments, at most 20% of the betalain pigment molecules is betanidin. It will be understood, that except when discussing the betanidin to iso-betanidin ratio and relationship, the general term betanidin is used so as to encompass both betanidin and iso-betanidin. In some embodiments, betanidin is betanidin and iso-betanidin.

In some embodiments, at most 1, 2, 5, 7, 10, 12, 15, 17, 20, 25, 30, 35, 40, 45, or 50% of the betalain pigment molecules is betanidin and/or iso-betanidin. Each possibility represents a separate embodiment of the invention. In some embodiments, at most 40% of the betalain pigment molecules is betanidin and/or iso-betanidin. In some embodiments, at most 20% of the betalain pigment molecules is betanidin and/or iso-betanidin.

In some embodiments, at most 60, 65, 70, 71, 72, 73, 74, 75, 76, 77, 78, 79, 80, 85, 90, 95 or 99% of the betalain pigment molecules is betanin. Each possibility represents a separate embodiment of the invention. In some embodiments, at most 60% of the betalain pigment molecules is betanin. In some embodiments, at most 75% of the betalain pigment molecules is betanin. In some embodiments, at most 80% of the betalain pigment molecules is betanin.

In some embodiments, at most 60, 65, 70, 71, 72, 73, 74, 75, 76, 77, 78, 79, 80, 85, 90, 95 or 99% of the betalain pigment molecules is betanin and/or iso-betanin. Each possibility represents a separate embodiment of the invention. In some embodiments, at most 60% of the betalain pigment molecules is betanin and/or iso-betanin. In some embodiments, at most 80% of the betalain pigment molecules is betanin and/or iso-betanin. In some embodiments, at most 85% of the betalain pigment molecules is betanin and/or iso-betanin. In some embodiments, at most 90% of the betalain pigment molecules is betanin and/or iso-betanin. In some embodiments, at most 99% of the betalain pigment molecules is betanin and/or iso-betanin.

In some embodiments, betanidin is between 0.5 and 99%, 0.5 and 95%, 0.5 and 90%, 0.5 and 85%, 0.5 and 80%, 0.5 and 75%, 0.5 and 70%, 0.5 and 65%, 0.5 and 60%, 0.5 and 55%, 0.5 and 50%, 0.5 and 45%, 0.5 and 40%, 0.5 and 35%, 0.5 and 30%, 0.5 and 25%, 0.5 and 20%, 0.5 and 15%, 0.5 and 10%, 1 and 99%, 1 and 95%, 1 and 90%, 1 and 85%, 1 and 80%, 1 and 75%, 1 and 70%, 1 and 65%, 1 and 60%, 1 and 55%, 1 and 50%, 1 and 45%, 1 and 40%, 1 and 35%, 1 and 30%, 1 and 25%, 1 and 20%, 1 and 15%, 1 and 10%, 5 and 99%, 5 and 95%, 5 and 90%, 5 and 85%, 5 and 80%, 5 and 75%, 5 and 70%, 5 and 65%, 5 and 60%, 5 and 55%, 5 and 50%, 5 and 45%, 5 and 40%, 5 and 35%, 5 and 30%, 5 and 25%, 5 and 20%, 5 and 15%, 5 and 10%, 10 and 99%, 10 and 95%, 10 and 90%, 10 and 85%, 10 and 80%, 10 and 75%, 10 and 70%, 10 and 65%, 10 and 60%, 10 and 55%, 10 and 50%, 10 and 45%, 10 and 40%, 10 and 35%, 10 and 30%, 10 and 25%, 10 and 20%, 10 and 15%, 15 and 99%, 15 and 95%, 15 and 90%, 15 and 85%, 15 and 80%, 15 and 75%, 15 and 70%, 15 and 65%, 15 and 60%, 15 and 55%, 15 and 50%, 15 and 45%, 15 and 40%, 15 and 35%, 15 and 30%, 15 and 25%, 15 and 20%, 20 and 99%, 20 and 95%, 20 and 90%, 20 and 85%, 20 and 80%, 20 and 75%, 20 and 70%, 20 and 65%, 20 and 60%, 20 and 55%, 20 and 50%, 20 and 45%, 20 and 40%, 20 and 35%, 20 and 30%, or 20 and 25% of the betalain pigment molecules in the composition. Each possibility represents a separate embodiment of the invention. In some embodiments, betanidin is between 1% and 90% of the betalain pigment molecules in the composition. In some embodiments, betanidin is between 10% and 90% of the betalain pigment molecules in the composition. In some embodiments, betanidin is between 1% and 75% of the betalain pigment molecules in the composition. In some embodiments, betanidin is between 10% and 75% of the betalain pigment molecules in the composition. In some embodiments, betanidin is between 1% and 10% of the betalain pigment molecules in the composition. In some embodiments, betanidin is between 1% and 5% of the betalain pigment molecules in the composition. In some embodiments, betanidin is between 1% and 2% of the betalain pigment molecules in the composition. In some embodiments, the percentages are of the betacyanin pigment molecules in the composition.

In some embodiments, betanin is between 1 and 99%, 1 and 95%, 1 and 90%, 1 and 85%, 1 and 80%, 1 and 75%, 1 and 70%, 1 and 65%, 1 and 60%, 1 and 55%, 1 and 50%, 1 and 45%, 1 and 40%, 1 and 35%, 1 and 30%, 1 and 25%, 1 and 20%, 1 and 15%, 1 and 10%, 5 and 99%, 5 and 95%, 5 and 90%, 5 and 85%, 5 and 80%, 5 and 75%, 5 and 70%, 5 and 65%, 5 and 60%, 5 and 55%, 5 and 50%, 5 and 45%, 5 and 40%, 5 and 35%, 5 and 30%, 5 and 25%, 5 and 20%, 5 and 15%, 5 and 10%, 10 and 99%, 10 and 95%, 10 and 90%, 10 and 85%, 10 and 80%, 10 and 75%, 10 and 70%, 10 and 65%, 10 and 60%, 10 and 55%, 10 and 50%, 10 and 45%, 10 and 40%, 10 and 35%, 10 and 30%, 10 and 25%, 10 and 20%, 10 and 15%, 15 and 99%, 15 and 95%, 15 and 90%, 15 and 85%, 15 and 80%, 15 and 75%, 15 and 70%, 15 and 65%, 15 and 60%, 15 and 55%, 15 and 50%, 15 and 45%, 15 and 40%, 15 and 35%, 15 and 30%, 15 and 25%, 15 and 20%, 20 and 99%, 20 and 95%, 20 and 90%, 20 and 85%, 20 and 80%, 20 and 75%, 20 and 70%, 20 and 65%, 20 and 60%, 20 and 55%, 20 and 50%, 20 and 45%, 20 and 40%, 20 and 35%, 20 and 30%, 20 and 25%, 25 and 99%, 25 and 95%, 25 and 90%, 25 and 85%, 25 and 80%, 25 and 75%, 25 and 70%, 25 and 65%, 25 and 60%, 25 and 55%, 25 and 50%, 25 and 45%, 25 and 40%, 25 and 35%, 25 and 30%, 30 and 99%, 30 and 95%, 30 and 90%, 30 and 85%, 30 and 80%, 30 and 75%, 30 and 70%, 30 and 65%, 30 and 60%, 30 and 55%, 30 and 50%, 30 and 45%, 30 and 40%, 30 and 35%, 35 and 99%, 35 and 95%, 35 and 90%, 35 and 85%, 35 and 80%, 35 and 75%, 35 and 70%, 35 and 65%, 35 and 60%, 35 and 55%, 35 and 50%, 35 and 45%, 35 and 40%, 40 and 99%, 40 and 95%, 40 and 90%, 40 and 85%, 40 and 80%, 40 and 75%, 40 and 70%, 40 and 65%, 40 and 60%, 40 and 55%, 40 and 50%, 40 and 45%, 45 and 99%, 45 and 95%, 45 and 90%, 45 and 85%, 45 and 80%, 45 and 75%, 45 and 70%, 45 and 65%, 45 and 60%, 45 and 55%, 45 and 50%, 1 and 99%, 50 and 95%, 50 and 90%, 50 and 85%, 50 and 80%, 50 and 75%, 50 and 70%, 50 and 65%, 50 and 60%, 50 and 55%, 55 and 99%, 55 and 95%, 55 and 90%, 55 and 85%, 55 and 80%, 55 and 75%, 55 and 70%, 55 and 65%, 55 and 60%, 60 and 99%, 60 and 95%, 60 and 90%, 60 and 85%, 60 and 80%, 60 and 75%, 60 and 70%, or 60 and 65% of the betalain pigment molecules in the composition. Each possibility represents a separate embodiment of the invention. In some embodiments, betanin is between 1% and 99% of the betalain pigment molecules in the composition. In some embodiments, betanin is between 10% and 99% of the betalain pigment molecules in the composition. In some embodiments, betanin is between 50% and 99% of the betalain pigment molecules in the composition. In some embodiments, betanin is between 1% and 90% of the betalain pigment molecules in the composition. In some embodiments, betanin is between 10% and 90% of the betalain pigment molecules in the composition. In some embodiments, betanin is between 50% and 90% of the betalain pigment molecules in the composition. In some embodiments, betanin is between 1% and 75% of the betalain pigment molecules in the composition. In some embodiments, betanin is between 10% and 75% of the betalain pigment molecules in the composition. In some embodiments, betanin is between 50% and 75% of the betalain pigment molecules in the composition. In some embodiments, betanin is between 80% and 99% of the betalain pigment molecules in the composition. In some embodiments, betanin is between 90% and 99% of the betalain pigment molecules in the composition. In some embodiments, betanin is between 95% and 99% of the betalain pigment molecules in the composition. In some embodiments, betanin is between 98% and 99% of the betalain pigment molecules in the composition. In some embodiments, the percentages are of the betacyanin pigment molecules in the composition.

In some embodiments, the ratio of betanidin to iso-betanidin in the composition is at least 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 12:1, 15:1, 17:1, 20:1, or 23:1. Each possibility represents a separate embodiment of the invention. In some embodiments, the ratio of betanidin to iso-betanidin in the composition is at least 1.5:1. In some embodiments, the ratio of betanidin to iso-betanidin in the composition is at least 2:1. In some embodiments, the ratio of betanidin to iso-betanidin in the composition is at least 5:1. In some embodiments, the ratio of betanidin to iso-betanidin in the composition is at least 7:1. In some embodiments, the ratio of betanidin to iso-betanidin in the composition is at least 7.5:1. In some embodiments, the ratio of betanidin to iso-betanidin in the composition is at least 10:1. In some embodiments, the ratio of betanidin to iso-betanidin in the composition is at least 20:1.

In some embodiments, the ratio of betanin to iso-betanin in the composition is at least 2:1, 3:1, 4:1, 5:1, 6:1, 7:1, 8:1, 9:1, 10:1, 12:1, 15:1, 17:1, 20:1, 22:1, 25:1, 27:1, 30:1, 32:1, 35:1, 37:1, 40:1, 42:1, 45:1, 47:1 or 50:1. Each possibility represents a separate embodiment of the invention. In some embodiments, the ratio of betanin to iso-betanin in the composition is at least 1.5:1. In some embodiments, the ratio of betanin to iso-betanin in the composition is at least 2:1. In some embodiments, the ratio of betanin to iso-betanin in the composition is at least 5:1. In some embodiments, the ratio of betanin to iso-betanin in the composition is at least 7:1. In some embodiments, the ratio of betanin to iso-betanin in the composition is at least 7.5:1. In some embodiments, the ratio of betanin to iso-betanin in the composition is at least 10:1. In some embodiments, the ratio of betanin to iso-betanin in the composition is at least 20:1. In some embodiments, the ratio of betanin to iso-betanin in the composition is at least 40:1.

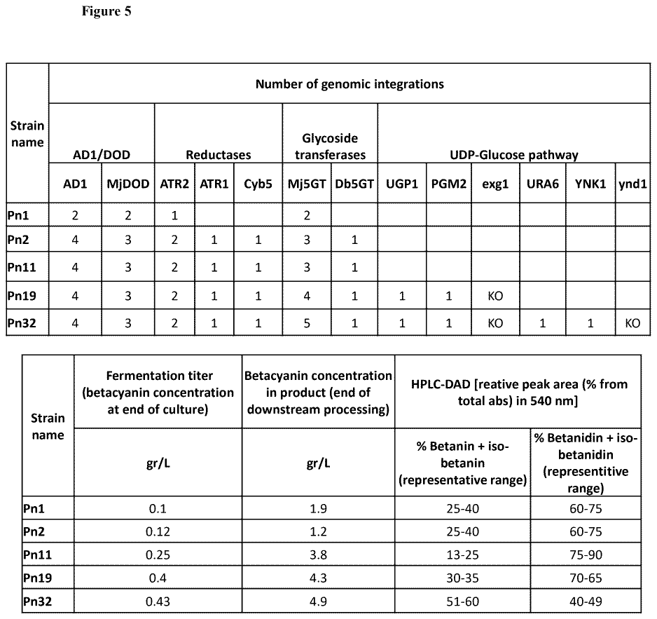

In some embodiments, the composition comprises a betalain pigment composition such as is provided in FIG. 5. In some embodiments, the composition comprises a betacyanin pigment composition such as is provided in FIG. 5. In some embodiments, the composition comprises the pigment percentages of Pn1. In some embodiments, the composition comprises the pigment percentages of Pn2. In some embodiments, the composition comprises the pigment percentages of Pn11. In some embodiments, the composition comprises the pigment percentages of Pn19. In some embodiments, the composition comprises the pigment percentages of Pn32. In some embodiments, pigment percentages are betalain pigment percentages. In some embodiments, pigment percentages are betacyanin pigment percentages.

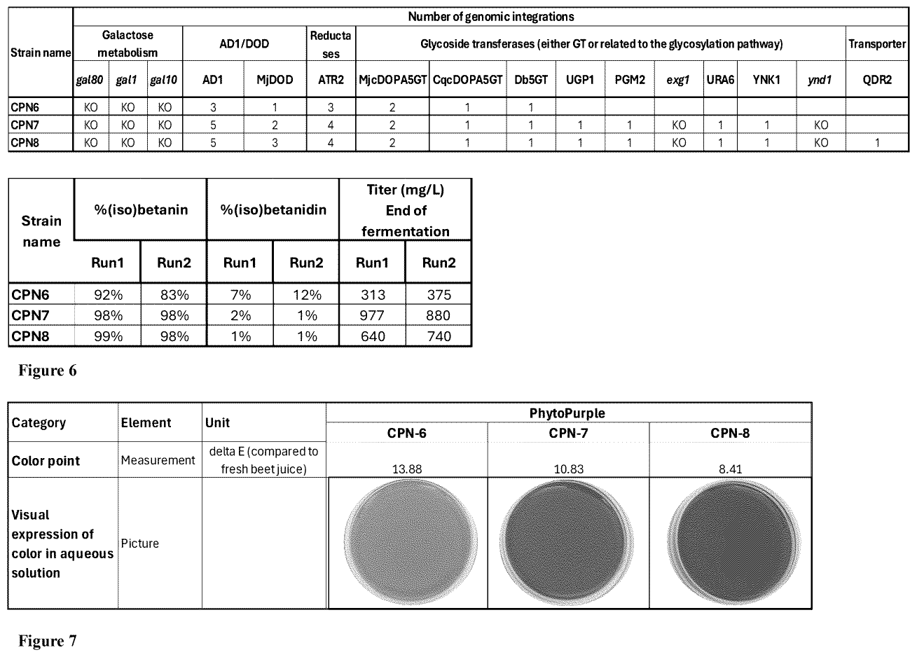

In some embodiments, the composition comprises a betalain pigment composition such as is provided in FIG. 6. In some embodiments, the composition comprises a betacyanin pigment composition such as is provided in FIG. 6. In some embodiments, the composition comprises the pigment percentages of CPN6. In some embodiments, the composition comprises the pigment percentages of CPN7. In some embodiments, the composition comprises the pigment percentages of CPN8. In some embodiments, pigment percentages are betalain pigment percentages. In some embodiments, pigment percentages are betacyanin pigment percentages.

In some embodiments, the composition comprises less than 20, 19, 18, 17, 16, 15, 14, 13, 12, 11, 10, 9, 8, 7, 6, 5, 4, 3, 2 or 1% betaxanthins. Each possibility represents a separate embodiment of the invention. In some embodiments, the composition comprises less than 10% betaxanthins. In some embodiments, the composition comprises less than 6% betaxanthins. In some embodiments, the composition comprises less than 5% betaxanthins. In some embodiments, the composition comprises less than 1% betaxanthins. In some embodiments, the betalain pigment molecules comprise less than 20, 19, 18, 17, 16, 15, 14, 13, 12, 11, 10, 9, 8, 7, 6, 5, 4, 3, 2 or 1% betaxanthins. Each possibility represents a separate embodiment of the invention. In some embodiments, the betalain pigment molecules comprise less than 10% betaxanthins. In some embodiments, the betalain pigment molecules comprise less than 6% betaxanthins. In some embodiments, the betalain pigment molecules comprise less than 5% betaxanthins. In some embodiments, the betalain pigment molecules comprise less than 1% betaxanthins. In some embodiments, betaxanthins is vulgaxanthin. In some embodiments, vulgaxanthin is vulgaxanthin I. In some embodiments, vulgaxanthin is vulgaxanthin I and/or vulgaxanthin II. In some embodiments, a betaxanthin comprises a betalamic acid conjugated to an amine. In some embodiments, the amine is an amino acid.

In some embodiments, the composition comprises less than 20, 19, 18, 17, 16, 15, 14, 13, 12, 11, 10, 9, 8, 7, 6, 5, 4, 3, 2 or 1% neobetanin. Each possibility represents a separate embodiment of the invention. In some embodiments, the composition comprises less than 10% neobetanin. In some embodiments, the composition comprises less than 6% neobetanin. In some embodiments, the composition comprises less than 5% neobetanin. In some embodiments, the composition comprises less than 1% neobetanin. In some embodiments, the betalain pigment molecules comprise less than 20, 19, 18, 17, 16, 15, 14, 13, 12, 11, 10, 9, 8, 7, 6, 5, 4, 3, 2 or 1% neobetanin. Each possibility represents a separate embodiment of the invention. In some embodiments, the betalain pigment molecules comprise less than 10% neobetanin. In some embodiments, the betalain pigment molecules comprise less than 6% neobetanin. In some embodiments, the betalain pigment molecules comprise less than 5% neobetanin. In some embodiments, the betalain pigment molecules comprise less than 1% neobetanin. Neobetanin is a degradation product with a brownish color. It is thus highly undesirable in a red pigment composition.

In some embodiments, the composition is low in sugar. In some embodiments, sugar is simple sugars. In some embodiments, simple sugars comprise monosaccharides and disaccharides. In some embodiments, simple sugars are monosaccharides. In some embodiments, simple sugars are disaccharides. In some embodiments, a composition low in sugar comprises less than 25, 23, 20, 18, 15, 13, 12, 11, 10, 9, 8, 7, 6, 5, 4, 3, 2, 1, 0.5, 0.1, 0.05 or 0.01% sugar. Each possibility represents a separate embodiment of the invention. In some embodiments, the composition comprises less than 10% sugar. In some embodiments, the composition comprises less than 5% sugar. In some embodiments, the composition comprises less than 1% sugar. In some embodiments, the percentage is by weight. In some embodiments, the composition is devoid of sugar. In some embodiments, the composition is essentially devoid of sugar.

In some embodiments, the composition is low in carbohydrates. In some embodiments, carbohydrates are dietary carbohydrates. In some embodiments, carbohydrates do not comprise betalains. In some embodiments, carbohydrates are non-betalain carbohydrates. In some embodiments, carbohydrates are carbohydrates that can be processed by humans to produce energy. It will be understood by a skilled artisan that while many molecules are technically carbohydrates, dietary carbohydrates are those that a human digestive tract can digest and convert into energy (calories). In some embodiments, a composition low in carbohydrates comprises less than 40, 35, 30, 27, 25, 23, 20, 19, 18, 17, 16, 15, 14, 13, 12, 11, 10, 9, 8, 7, 6, 5, 4, 3, 2, 1, 0.5, 0.1, or 0.05% carbohydrates. Each possibility represents a separate embodiment of the invention. In some embodiments, the composition comprises less than 10% carbohydrates. In some embodiments, the composition comprises less than 15% carbohydrates. In some embodiments, the percentage is by weight.

In some embodiments, the composition comprises at least 500, 550, 600, 650, 700, 750, 800, 850, 900, 950, 1000, 1050, 1100, 1200, 1300, 1400 or 1500 mg magnesium per liter. Each possibility represents a separate embodiment of the invention. In some embodiments, the composition comprises at least 500 mg magnesium per liter. In some embodiments, the composition is a liquid and comprises at least 500 mg magnesium per liter. In some embodiments, the composition comprises at least 1000 mg magnesium per liter. In some embodiments, the composition is a liquid and comprises at least 1000 mg magnesium per liter. In some embodiments, the composition comprises at least 500, 550, 600, 650, 700, 750, 800, 850, 900, 950, 1000, 1050, 1100, 1200, 1300, 1400, 1500, 1600, 1700, 1800, 1900 or 2000 mg magnesium per kilogram. Each possibility represents a separate embodiment of the invention. In some embodiments, the composition comprises at least 500 mg magnesium per kilogram. In some embodiments, the composition is a solid and comprises at least 500 mg magnesium per kilogram. In some embodiments, the composition comprises at least 1000 mg magnesium per kilogram. In some embodiments, the composition is a solid and comprises at least 1000 mg magnesium per kilogram.

In some embodiments, the composition comprises at most 100, 90, 80, 75, 70, 60, 50, 45, 40, 35, 30, 25, 20 15 or 10 mg sodium per liter. Each possibility represents a separate embodiment of the invention. In some embodiments, the composition comprises at most 50 mg sodium per liter. In some embodiments, the composition is a liquid and comprises at most 50 mg sodium per liter. In some embodiments, the composition comprises at most 100 mg sodium per liter. In some embodiments, the composition is a liquid and comprises at most 100 mg sodium per liter. In some embodiments, the composition comprises at most 100, 90, 80, 75, 70, 60, 50, 45, 40, 35, 30, 25, 20 15 or 10 mg sodium per kilogram. Each possibility represents a separate embodiment of the invention. In some embodiments, the composition comprises at most 50 mg sodium per kilogram. In some embodiments, the composition is a solid and comprises at most 50 mg sodium per kilogram. In some embodiments, the composition comprises at most 100 mg sodium per kilogram. In some embodiments, the composition is a solid and comprises at most 100 mg sodium per kilogram.

In some embodiments, the composition comprises less than 5, 4, 3, 2, 1.5, 1, 0.9, 0.8, 0.7, 0.6, 0.5, 0.4, 0.3, 0.2, 0.1, 0.05, or 0.01 μg/kg geosmin. Each possibility represents a separate embodiment of the invention. In some embodiments, the composition comprises less than 0.01 μg/kg geosmin. In some embodiments, the composition comprises less than 0.1 μg/kg geosmin. In some embodiments, the composition comprises less than 1 μg/kg geosmin. In some embodiments, the composition is essentially devoid of geosmin. In some embodiments, the composition is a geosmin-free composition.

In some embodiments, the composition comprises less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0005 or 0.0001 g/L nitrates. Each possibility represents a separate embodiment of the invention. In some embodiments, the composition comprises less than 0.01 g/L nitrates. In some embodiments, the composition comprises less than 0.005 g/L nitrates. In some embodiments, the composition comprises less than 0.001 g/L nitrates. In some embodiments, the composition is essentially devoid of nitrates. In some embodiments, the composition is a nitrate-free composition.

In some embodiments, the composition is a high yield composition. In some embodiments, the composition is a high titer composition. In some embodiments, yield is concentration. In some embodiments, the composition comprises a higher titer of betalains than are found in nature. In some embodiments, in nature is in a beetroot. In some embodiments, in a beetroot is in a beetroot extract. In some embodiments, in a beetroot is in beetroot juice. In some embodiments, a beetroot extract is a betalain beetroot extract. In some embodiments, the composition comprises a betalain concentration of greater than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, or 5 grams betalain pigment molecules per liter of composition. Each possibility represents a separate embodiment of the invention. In some embodiments, the composition comprises a betalain concentration of greater than 0.05 grams betalain pigment molecules per liter of composition. In some embodiments, the composition comprises a betalain concentration of greater than 0.5 grams betalain pigment molecules per liter of composition. In some embodiments, the composition comprises a betalain concentration of greater than 0.75 grams betalain pigment molecules per liter of composition. In some embodiments, the composition comprises a betalain concentration of greater than 1 gram betalain pigment molecules per liter of composition. In some embodiments, the composition comprises a betalain concentration of greater than 1.5 grams betalain pigment molecules per liter of composition. In some embodiments, the composition comprises a betalain concentration of greater than 3 grams betalain pigment molecules per liter of composition. In some embodiments, the composition comprises a betalain concentration of greater than 3.5 grams betalain pigment molecules per liter of composition. In some embodiments, the composition comprises a betalain concentration of greater than 4 grams betalain pigment molecules per liter of composition. It will be understood by a skilled artisan that for commercially relevant production of pigment a concentration of at least about 4 gr/l is needed, thus the highly concentrated compositions of the invention are highly advantageous. Such a high concentration of pigment from fermentation has not previously been reported.

In some embodiments, the composition comprises a betalain concentration of greater than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5 or 4 grams betalain pigment molecules per kilogram of composition. Each possibility represents a separate embodiment of the invention. In some embodiments, the composition comprises a betalain concentration of greater than 0.05 grams betalain pigment molecules per kilogram of composition. In some embodiments, the composition comprises a betalain concentration of greater than 0.5 grams betalain pigment molecules per kilogram of composition. In some embodiments, the composition comprises a betalain concentration of greater than 0.75 grams betalain pigment molecules per kilogram of composition. In some embodiments, the composition comprises a betalain concentration of greater than 1 gram betalain pigment molecules per kilogram of composition. In some embodiments, the composition comprises a betalain concentration of greater than 1.5 grams betalain pigment molecules per kilogram of composition. In some embodiments, the composition comprises a betalain concentration of greater than 3 grams betalain pigment molecules per kilogram of composition. In some embodiments, the composition comprises a betalain concentration of greater than 3.5 grams betalain pigment molecules per kilogram of composition. In some embodiments, the composition comprises a betalain concentration of greater than 4 grams betalain pigment molecules per kilogram of composition.

In some embodiments, the composition is devoid of intact cells. In some embodiments, devoid of is essentially devoid of. In some embodiments, cells are plant cells. In some embodiments, cells are yeast cells. In some embodiments, the cells are bacterial cells. In some embodiments, the composition is devoid of plant proteins not of the betalain synthesis pathway.

In some embodiments, the composition is an artificial composition. In some embodiments, the composition is not found in nature. In some embodiments, the composition comprises a ratio of betanin to betanidin not found in nature. In some embodiments, the composition comprises a carrier. In some embodiments, the composition comprises a bulking agent. In some embodiments, the carrier is an additive. In some embodiments, the carrier is a sugar. In some embodiments, the bulking agent is a sugar. In some embodiments, the sugar is maltodextrin. In some embodiments, maltodextrin is maltodextrin DE-18. In some embodiments, maltodextrin is maltodextrin DE-12. In some embodiments, maltodextrin is maltodextrin DE-9. In some embodiments, the bulking agent is whey protein concentrate. In some embodiments, the additive is whey protein concentrate. In some embodiments, the additive is whey protein isolate. In some embodiments, whey protein concentrate comprises or consists of α-lactalbumin, β-lactoglobulin, serum, albumin and immunoglobulins. Bulking agents and carriers are well known in the art and any such compounds may be used. Further examples of bulking agents and carriers include, but are not limited to cyclodextrins, dextrose/polydextrose, silicon dioxide, starches/modified starches, and cellulose. In some embodiments, the composition comprises a thickener. In some embodiments, the composition comprises a stabilizer. In some embodiments, the thicken or the stabilizer is tara gum.

By another aspect, there is provided a cell comprising at least one of the following genes integrated into its genome: a tyrosine hydroxylase gene, an L-DOPA oxidase gene, a DOPA 4,5-dioxygenase (DOD) gene, a NADPH cytochrome P450 reductase gene, a 5-O-betanidin-glycosyltransferase and a cyclo-DOPA-5-O-glucosyltransferase gene.

By another aspect, there is provided a cell comprising integrated into its genome at least one gene selected from: a tyrosine hydroxylase gene, a L-DOPA oxidase gene, a DOPA 4,5-dioxygenase (DOD) gene, a NADPH cytochrome P450 reductase gene, a 5-O-betanidin-glycosyltransferase and a cyclo-DOPA-5-O-glucosyltransferase gene.

In some embodiments, the cell comprises at least one of the following genes integrated into its genome: a tyrosine hydroxylase gene, an L-DOPA oxidase gene, a DOPA 4,5-dioxygenase (DOD) gene, a NADPH cytochrome P450 reductase gene and a cyclo-DOPA-5-O-glucosyltransferase gene. In some embodiments, the cell comprises integrated into its genome at least one gene selected from: a tyrosine hydroxylase gene, a L-DOPA oxidase gene, a DOPA 4,5-dioxygenase (DOD) gene, a NADPH cytochrome P450 reductase gene and a cyclo-DOPA-5-O-glucosyltransferase gene.

Methods of gene integration into a cell's genome are well known in the art and any such method may be used. Examples include use of homologous recombination, CRISPR-Cas9, TALE nucleases, and zinc-finger nucleases as well as lentiviral and adenoviral integration. In some embodiments, the integration is viral integration. In some embodiments, the integration is adenoviral integration. In some embodiments, the integration is by homologous recombination. In some embodiments, the integration is by a guided nuclease. In some embodiments, the guided nuclease is CRISPR-Cas. In some embodiments, the guided nuclease is CRISPR-Cas9. In some embodiments, the integration is by CRISPR-Cas9.

In some embodiments, the genes are exogenous genes. In some embodiments, the genes are not endogenous to the cell. In some embodiments, the genes lack introns. In some embodiments, the genes are cDNAs integrated into the cell's genome. In some embodiments, the genes are plant genes. In some embodiments, the cell is not a plant cell. In some embodiments, the cell is a plant cell. In some embodiments, the cell is a plant cell that does not express betalains. In some embodiments, the plant cell does not endogenously express betalains. In some embodiments, the cell is a bacterial cell. In some embodiments, the bacterial cell is E. coli. In some embodiments, the cell is a eukaryotic cell. In some embodiments, the cell is a fungal cell. In some embodiments, the fungus is a unicellular fungus. In some embodiments, the fungus is a filamentous fungus. In some embodiments, the fungus is not a filamentous fungus. In some embodiments, the fungus is yeast. In some embodiments, the cell is a yeast cell. In some embodiments, the yeast is Saccharomyces cerevisiae.

In some embodiments, at least one gene is at least two genes. In some embodiments, at least one gene is at least three genes. In some embodiments, at least one gene is at least four genes. In some embodiments, the cell comprises integrated into its genome all of a tyrosine hydroxylase gene, a L-DOPA oxidase gene, a DOPA 4,5-dioxygenase (DOD) gene, a NADPH cytochrome P450 reductase gene, and a cyclo-DOPA-5-O-glucosyltransferase gene. In some embodiments, a gene is integrated at least once. In some embodiments, at least one copy of the gene is integrated. In some embodiments, at least one copy of a tyrosine hydroxylase gene is integrated. In some embodiments, at least one copy of a L-DOPA oxidase gene is integrated. In some embodiments, at least one copy of a DOPA 4, 5-dioxygenase (DOD) gene is integrated. In some embodiments, at least one copy of a NADPH cytochrome P450 reductase gene is integrated. In some embodiments, at least one copy of a cyclo-DOPA-D-O-glucosyltransferase gene is integrated. In some embodiments, at least one copy of a 5-O-betanidin-glycosyltransferase gene is integrated.

In some embodiments, the tyrosine hydroxylase gene and the L-DOPA oxidase gene are the same gene. In some embodiments, the tyrosine hydroxylase gene is CYP76AD1. In some embodiments, the L-DOPA oxidase gene is CYP76AD1. In some embodiments, CYP76AD1 is Beta vulgaris CYP76AD1. In some embodiments, the nucleic acid sequence encoding CYP76AD1 comprises SEQ ID NO: 1. In some embodiments, the gene is without introns. In some embodiments, the gene is a cDNA of the gene. In some embodiments, the tyrosine hydroxylase gene comprises SEQ ID NO: 1. In some embodiments, the tyrosine hydroxylase gene consists of SEQ ID NO: 1. In some embodiments, the L-DOPA oxidase gene comprises SEQ ID NO: 1. In some embodiments, the L-DOPA oxidase gene consists of SEQ ID NO: 1. In some embodiments, the tyrosine hydroxylase gene comprises a sequence with at least 70, 75, 80, 85, 90, 95, 97 or 99% identity to SEQ ID NO: 1 while retaining tyrosine hydroxylase activity. Each possibility represents a separate embodiment of the invention. In some embodiments, the L-DOPA oxidase gene comprises a sequence with at least 70, 75, 80, 85, 90, 95, 97 or 99% identity to SEQ ID NO: 1 while retaining L-DOPA oxidase activity. Each possibility represents a separate embodiment of the invention. In some embodiments, retaining activity is encodes a protein that retains activity. Examples of genes that are both tyrosine hydroxylases and L-DOPA oxidases include, but are not limited to, Amaranthus cruentus CYP76AD2 (accession AET43291.1) Mirabilis jalapa CYP76AD3 (accession AET43292.1), and Celosia cristata CYP76AD4 (accession AGI78466.1). In some embodiments, the tyrosine hydroxylase is selected from CYP76AD1, CYP76AD2, CYP76AD3, and CYP76AD4. In some embodiments, the L-DOPA hydroxylase is selected from CYP76AD1, CYP76AD2, CYP76AD3, and CYP76AD4.

In some embodiments, the amino acid sequence of CYP76AD1 comprises SEQ ID NO: 2. In some embodiments, the amino acid sequence of CYP76AD1 consists of SEQ ID NO: 2. In some embodiments, the CYP76AD1 gene comprises a nucleic acid sequence that encodes SEQ ID NO: 2. In some embodiments, the CYP76AD1 gene consists of a nucleic acid sequence that encodes SEQ ID NO: 2. In some embodiments, the CYP76AD1 gene comprises or consists of a nucleic acid sequence that encodes a protein with at least 70, 75, 80, 85, 90, 95, 97 or 99% identity to SEQ ID NO: 2. Each possibility represents a separate embodiment of the invention. In some embodiments, the protein retains hydroxylase activity.

In some embodiments, the protein retains oxidase activity.

In some embodiments, the DOD gene is Mirabilis jalapa DOD (MjDOD). In some embodiments, the DOD gene is Beta vulgaris DODA1 (BvDODA1). In some embodiments, the nucleic acid sequence encoding MjDOD comprises SEQ ID NO: 3. In some embodiments, the nucleic acid sequence encoding BvDODA1 comprises SEQ ID NO: 5. In some embodiments, the gene is without introns. In some embodiments, the gene is a cDNA of the gene. In some embodiments, the DOPA 4,5-dioxygenase gene comprises SEQ ID NO: 3. In some embodiments, the DOPA 4,5-dioxygenase gene consists of SEQ ID NO: 3. In some embodiments, the DOPA 4,5-dioxygenase gene comprises SEQ ID NO: 5. In some embodiments, the DOPA 4,5-dioxygenase gene consists of SEQ ID NO: 5. In some embodiments, the DOPA 4,5-dioxygenase gene comprises a sequence with at least 70, 75, 80, 85, 90, 95, 97 or 99% identity to SEQ ID NO: 3 while retaining dioxygenase activity. Each possibility represents a separate embodiment of the invention. In some embodiments, the DOPA 4,5-dioxygenase gene comprises a sequence with at least 70, 75, 80, 85, 90, 95, 97 or 99% identity to SEQ ID NO: 5 while retaining dioxygenase activity. Each possibility represents a separate embodiment of the invention. In some embodiments, retaining activity is encoding a protein that retains activity. Other examples of DOD genes include but are not limited to PgDOD from Portulaca grandiflora (Accession No. AJ580598), BgDOD from Bougainvillea glabra (Accession No. AB435373), and AmDOD from Amanita muscaria (Accession No. P87064).

In some embodiments, the amino acid sequence of MjDOD comprises SEQ ID NO: 4. In some embodiments, the amino acid sequence of MjDOD consists of SEQ ID NO: 4. In some embodiments, the MjDOD gene comprises a nucleic acid sequence that encodes SEQ ID NO: 4. In some embodiments, the MjDOD gene consists of a nucleic acid sequence that encodes SEQ ID NO: 4. In some embodiments, the MjDOD gene comprises or consists of a nucleic acid sequence that encodes a protein with at least 70, 75, 80, 85, 90, 95, 97 or 99% identity to SEQ ID NO: 4. Each possibility represents a separate embodiment of the invention. In some embodiments, the protein retains dioxygenase activity.

In some embodiments, the amino acid sequence of BvDODA1 comprises SEQ ID NO: 6. In some embodiments, the amino acid sequence of BvDODA1 consists of SEQ ID NO: 6. In some embodiments, the BvDODA1 gene comprises a nucleic acid sequence that encodes SEQ ID NO: 6. In some embodiments, the BvDODA1 gene consists of a nucleic acid sequence that encodes SEQ ID NO: 6. In some embodiments, the BvDODA1 gene comprises or consists of a nucleic acid sequence that encodes a protein with at least 70, 75, 80, 85, 90, 95, 97 or 99% identity to SEQ ID NO: 6. Each possibility represents a separate embodiment of the invention. In some embodiments, the protein retains dioxygenase activity.

In some embodiments, the NADPH cytochrome P450 reductase gene is NADPH cytochrome P450 reductase 2 (ATR2) from Arabidopsis thaliana. In some embodiments, the NADPH cytochrome P450 reductase gene is NADPH-cytochrome P450 reductase 1 (ATR1) from Arabidopsis thaliana. In some embodiments, the cell comprises at least one copy of the ATR1 gene integrated into its genome. In some embodiments, the cell comprises at least one copy of the ATR2 gene integrated into its genome. In some embodiments, the nucleic acid sequence encoding ATR1 comprises SEQ ID NO: 7. In some embodiments, an optimized nucleic acid sequence encoding ATR1 comprises SEQ ID NO: 29. In some embodiments, optimized is codon optimized. In some embodiments, optimized is optimized to remove restriction sites. In some embodiments, optimized is optimized for expression in a cell. In some embodiments, the nucleic acid sequence encoding ATR2 comprises SEQ ID NO: 9. In some embodiments, an optimized nucleic acid sequence encoding ATR2 comprises SEQ ID NO: 30. In some embodiments, optimized is codon optimized. In some embodiments, optimized is optimized for expression in a cell. In some embodiments, the gene is without introns. In some embodiments, the gene is a cDNA of the gene. In some embodiments, the NADPH cytochrome P450 reductase gene comprises SEQ ID NO: 7. In some embodiments, the NADPH cytochrome P450 reductase gene consists of SEQ ID NO: 7. In some embodiments, the NADPH cytochrome P450 reductase gene comprises SEQ ID NO: 9. In some embodiments, the NADPH cytochrome P450 reductase gene consists of SEQ ID NO: 9. In some embodiments, the DOPA 4,5-dioxygenase gene comprises a sequence with at least 70, 75, 80, 85, 90, 95, 97 or 99% identity to SEQ ID NO: 7 while retaining reductase activity. Each possibility represents a separate embodiment of the invention. In some embodiments, the DOPA 4,5-dioxygenase gene comprises a sequence with at least 70, 75, 80, 85, 90, 95, 97 or 99% identity to SEQ ID NO: 29 while retaining reductase activity.

Each possibility represents a separate embodiment of the invention. In some embodiments, the NADPH cytochrome P450 reductase gene comprises a sequence with at least 70, 75, 80, 85, 90, 95, 97 or 99% identity to SEQ ID NO: 9 while retaining reductase activity. Each possibility represents a separate embodiment of the invention. In some embodiments, the NADPH cytochrome P450 reductase gene comprises a sequence with at least 70, 75, 80, 85, 90, 95, 97 or 99% identity to SEQ ID NO: 30 while retaining reductase activity. Each possibility represents a separate embodiment of the invention. In some embodiments, retaining activity is encoding a protein that retains activity. Other examples of NADPH cytochrome P450 reductase genes include but are not limited to NADPH cytochrome P450 reductase 1 or 2 from Medicago truncatula (MtCPR1/MtCPR2) and NADPH cytochrome P450 reductase 1 or 2 from Lotus japonicus (LjCPR1/LjCPR2).

In some embodiments, the amino acid sequence of ATR1 comprises SEQ ID NO: 8. In some embodiments, the amino acid sequence of ATR1 consists of SEQ ID NO: 8. In some embodiments, the ATR1 gene comprises a nucleic acid sequence that encodes SEQ ID NO: 8. In some embodiments, the ATR1 gene consists of a nucleic acid sequence that encodes SEQ ID NO: 8. In some embodiments, the MjDOD gene comprises or consists of a nucleic acid sequence that encodes a protein with at least 70, 75, 80, 85, 90, 95, 97 or 99% identity to SEQ ID NO: 8. Each possibility represents a separate embodiment of the invention. In some embodiments, the protein retains reductase activity.

In some embodiments, the amino acid sequence of ATR2 comprises SEQ ID NO: 10. In some embodiments, the amino acid sequence of ATR2 consists of SEQ ID NO: 10. In some embodiments, the ATR2 gene comprises a nucleic acid sequence that encodes SEQ ID NO: 10. In some embodiments, the ATR2 gene consists of a nucleic acid sequence that encodes SEQ ID NO: 10. In some embodiments, the ATR2 gene comprises or consists of a nucleic acid sequence that encodes a protein with at least 70, 75, 80, 85, 90, 95, 97 or 99% identity to SEQ ID NO: 10. Each possibility represents a separate embodiment of the invention. In some embodiments, the protein retains reductase activity.

In some embodiments, a cyclo-DOPA-5-O-glucosyltransferase gene is a 5-O-cyclo-DOPA-glucosyltransferase gene. In some embodiments, the cyclo-DOPA-5-O-glucosyltransferase gene is Mirabilis jalapa cyclo-DOPA-5-O-glucosyltransferase (MjcDOPA5GT). In some embodiments, the nucleic acid sequence encoding MjcDOPA5GT comprises SEQ ID NO: 11. In some embodiments, the gene is without introns. In some embodiments, the gene is a cDNA of the gene. In some embodiments, the cyclo-DOPA-5-O-glucosyltransferase gene comprises SEQ ID NO: 11. In some embodiments, the cyclo-DOPA-5-O-glucosyltransferase gene consists of SEQ ID NO: 11. In some embodiments, the cyclo-DOPA-5-O-glucosyltransferase gene comprises a sequence with at least 70, 75, 80, 85, 90, 95, 97 or 99% identity to SEQ ID NO: 11 while retaining glucosyltransferase activity. Each possibility represents a separate embodiment of the invention. In some embodiments, retaining activity is encoding a protein that retains activity. In some embodiments, the cyclo-DOPA-5-O-glucosyltransferase gene is Chenopodium quinoa cyclo-DOPA-5-O-glucosyltransferase (CqcDOPA5GT). In some embodiments, the nucleic acid sequence encoding CqcDOPA5GT comprises SEQ ID NO: 42. In some embodiments, the gene is without introns. In some embodiments, the gene is a cDNA of the gene. In some embodiments, the cyclo-DOPA-5-O-glucosyltransferase gene comprises SEQ ID NO: 42. In some embodiments, the cyclo-DOPA-5-O-glucosyltransferase gene consists of SEQ ID NO: 42. In some embodiments, the cyclo-DOPA-5-O-glucosyltransferase gene comprises a sequence with at least 70, 75, 80, 85, 90, 95, 97 or 99% identity to SEQ ID NO: 42 while retaining glucosyltransferase activity. Each possibility represents a separate embodiment of the invention. In some embodiments, retaining activity is encoding a protein that retains activity. Other examples of cyclo-DOPA-5-O-glucosyltransferase genes include but are not limited to Celosia cristata cyclo-DOPA-5-O-glucosyltransferase (CccDOPA5GT). In some embodiments, the cyclo-DOPA-5-O-glucosyltransferase gene is selected from MjcDOPA5GT and CqcDOPA5GT. In some embodiments, the cyclo-DOPA-5-O-glucosyltransferase gene is MjcDOPA5GT, CqcDOPA5GT or both. Herein, the term “cDOPA5GT” is used to refer to either MjcDOPA5GT or CqcDOPA5GT.

In some embodiments, the amino acid sequence of MjcDOPA5GT comprises SEQ ID NO: 12. In some embodiments, the amino acid sequence of MjcDOPA5GT consists of SEQ ID NO: 12. In some embodiments, the MjcDOPA5GT gene comprises or consists of a nucleic acid sequence that encodes a protein with at least 70, 75, 80, 85, 90, 95, 97 or 99% identity to SEQ ID NO: 12. Each possibility represents a separate embodiment of the invention. In some embodiments, the protein retains glucosyltransferase activity. In some embodiments, the amino acid sequence of CqcDOPA5GT comprises SEQ ID NO: 43. In some embodiments, the amino acid sequence of CqcDOPA5GT consists of SEQ ID NO: 43. In some embodiments, the CqcDOPA5GT gene comprises or consists of a nucleic acid sequence that encodes a protein with at least 70, 75, 80, 85, 90, 95, 97 or 99% identity to SEQ ID NO: 43. Each possibility represents a separate embodiment of the invention. In some embodiments, the protein retains glucosyltransferase activity.

In some embodiments, the cell comprises at least two copies of the tyrosine hydroxylase gene. In some embodiments, the cell comprises at least two copies of the L-DOPA oxidase gene. In some embodiments, the cell comprises at least two copies of the DOD gene. In some embodiments, the cell comprises at least two copies of the NADPH cytochrome P450 reductase gene. In some embodiments, the cell comprises at least two copies of the cyclo-DOPA-5-O-glucosyltransferase gene. In some embodiments, at least two copies is at least two copies integrated into the genome of the cell. In some embodiments, at least two is at least 3, 4, 5, 6, 7, 8, 9 or 10. Each possibility represents a separate embodiment of the invention. In some embodiments, at least 2 is 2. In some embodiments, at least 2 is 3. In some embodiments, at least 2 is 4. In some embodiments, at least 2 is 5.

In some embodiments, the cell comprises at least two copies of CYP76AD1 integrated into its genome. In some embodiments, the cell comprises at least four copies of CYP76AD1 integrated into its genome. In some embodiments, the cell comprises at least two copies of MjDOD integrated into its genome. In some embodiments, the cell comprises at least three copies of MjDOD integrated into its genome. In some embodiments, the cell comprises at least two copies of ATR2 integrated into its genome. In some embodiments, the cell comprises at least two copies of MjcDOPA5GT integrated into its genome. In some embodiments, the cell comprises at least three copies of MjcDOPA5GT integrated into its genome. In some embodiments, the cell comprises at least four copies of MjcDOPA5GT integrated into its genome. In some embodiments, the cell comprises at least five copies of MjcDOPA5GT integrated into its genome. In some embodiments, the cell comprises at least two copies of cDOPA5GT integrated into its genome. In some embodiments, the cell comprises at least three copies of cDOPA5GT integrated into its genome. In some embodiments, the cell comprises at least four copies of cDOPA5GT integrated into its genome. In some embodiments, the cell comprises at least five copies of cDOPA5GT integrated into its genome.

In some embodiments, the cell comprises at least two copies of said exogenous tyrosine hydroxylase gene, at least two copies of said exogenous L-DOPA oxidase gene, at least one copy of said exogenous DOD gene, at least two copies of said exogenous reductase gene, and at least two copies of said exogenous glycosyl transferase gene integrated into the genome of the cell. In some embodiments, the cell comprises at least two copies of said exogenous tyrosine hydroxylase gene, at least two copies of said exogenous L-DOPA oxidase gene, at least two copies of said exogenous DOD gene, at least two copies of said exogenous reductase gene, and at least two copies of said exogenous glycosyl transferase gene integrated into the genome of the cell. In some embodiments, the cell comprises at least 3 copies of the exogenous tyrosine hydroxylase gene, at least 3 copies of the exogenous L-DOPA oxidase gene, at least 1 copy of the exogenous DOD gene, at least 3 copies of the exogenous reductase gene, and at least 3 copies of the exogenous glycosyl transferase gene integrated into the genome of the cell. In some embodiments, the cell comprises 3 copies of the exogenous tyrosine hydroxylase gene, 3 copies of the exogenous L-DOPA oxidase gene, 1 copy of the exogenous DOD gene, 3 copies of the exogenous reductase gene, and 3 copies of the exogenous glycosyl transferase gene integrated into the genome of the cell. In some embodiments, the cell comprises at least 5 copies of the exogenous tyrosine hydroxylase gene, at least 5 copies of the exogenous L-DOPA oxidase gene, at least 2 copies of the exogenous DOD gene, at least 4 copies of the exogenous reductase gene, and at least 3 copies of the exogenous glycosyl transferase gene integrated into the genome of the cell. In some embodiments, the cell comprises 5 copies of the exogenous tyrosine hydroxylase gene, 5 copies of the exogenous L-DOPA oxidase gene, 2 or 3 copies of the exogenous DOD gene, 4 copies of the exogenous reductase gene, and 3 copies of the exogenous glycosyl transferase gene integrated into the genome of the cell.

In some embodiments, the cell comprises at least two copies of a CYP76AD1 gene, 1 copy of a DOD gene, at least two copies of an NADPH cytochrome P450 reductase gene, at least 2 copies of a cyclo-DOPA-5-O-glucosyltransferase gene integrated into the genome of the cell. In some embodiments, the cell comprises at least 3 copies of CYP76AD1, at least 1 copy of a DOD gene, at least 3 copies of an NADPH cytochrome P450 reductase gene, and at least 3 copies of a cyclo-DOPA-5-O-glucosyltransferase gene integrated into the genome of the cell. In some embodiments, the cell comprises 3 copies of CYP76AD1, 1 copy of a DOD gene, 3 copies of an NADPH cytochrome P450 reductase gene, and 3 copies of a cyclo-DOPA-5-O-glucosyltransferase gene integrated into the genome of the cell. In some embodiments, the cell comprises at least 5 copies of CYP76AD1, at least 2 copies of a DOD gene, at least 4 copies of an NADPH cytochrome P450 reductase gene, and at least 3 copies of a cyclo-DOPA-5-O-glucosyltransferase gene integrated into the genome of the cell. In some embodiments, the cell comprises 5 copies of CYP76AD1, 2 or 3 copies of a DOD gene, 4 copies of an NADPH cytochrome P450 reductase gene, and 3 copies of a cyclo-DOPA-5-O-glucosyltransferase gene integrated into the genome of the cell.

In some embodiments, the cell comprises at least two copies of CYP76AD1, at least two copies of MjDOD, at least one copy of ATR2 and at least two copies of MjcDOPA5GT integrated into its genome. In some embodiments, the cell comprises at least two copies of CYP76AD1, at least two copies of MjDOD, at least one copy of ATR2 and at least two copies of cDOPA5GT integrated into its genome. In some embodiments, the cell comprises two copies of CYP76AD1, two copies of MjDOD, one copy of ATR2 and two copies of MjcDOPA5GT integrated into its genome. In some embodiments, the cell comprises at least two copies of CYP76AD1, at least two copies of MjDOD, at least one copy of ATR2 and at least two copies of cDOPA5GT integrated into its genome. In some embodiments, the cell comprises at least 3 copies of CYP76AD1, at least 1 copy of MjDOD, at least 3 copies of ATR2 and at least 3 copies of cDOPA5GT integrated into its genome. In some embodiments, at least 3 copies of cDOPA5GT comprises at least two copies of MjcDOPA5GT and at least one copy of CqcDOPA5GT. In some embodiments, the cell comprises 3 copies of CYP76AD1, one copy of MjDOD, 3 copy of ATR2 and 3 copies of cDOPA5GT integrated into its genome. In some embodiments, 3 copies of cDOPA5GT is two copies of MjcDOPA5GT and one copy of CqcDOPA5GT. In some embodiments, the cell comprises at least four copies of CYP76AD1, at least three copies of MjDOD, at least two copies of ATR2 and at least three copies of MjcDOPA5GT integrated into its genome. In some embodiments, the cell comprises at least four copies of CYP76AD1, at least three copies of MjDOD, at least two copies of ATR2 and at least three copies of cDOPA5GT integrated into its genome. In some embodiments, the cell comprises four copies of CYP76AD1, three copies of MjDOD, two copies of ATR2 and three copies of MjcDOPA5GT integrated into its genome. In some embodiments, the cell comprises four copies of CYP76AD1, three copies of MjDOD, two copies of ATR2 and three copies of cDOPA5GT integrated into its genome. In some embodiments, the cell comprises at least four copies of CYP76AD1, at least three copies of MjDOD, at least two copies of ATR2 and at least four copies of MjcDOPA5GT integrated into its genome. In some embodiments, the cell comprises at least four copies of CYP76AD1, at least three copies of MjDOD, at least two copies of ATR2 and at least four copies of cDOPA5GT integrated into its genome. In some embodiments, the cell comprises four copies of CYP76AD1, three copies of MjDOD, two copies of ATR2 and four copies of MjcDOPA5GT integrated into its genome. In some embodiments, the cell comprises four copies of CYP76AD1, three copies of MjDOD, two copies of ATR2 and four copies of cDOPA5GT integrated into its genome. In some embodiments, the cell comprises at least four copies of CYP76AD1, at least three copies of MjDOD, at least two copies of ATR2 and at least five copies of MjcDOPA5GT integrated into its genome. In some embodiments, the cell comprises at least four copies of CYP76AD1, at least three copies of MjDOD, at least two copies of ATR2 and at least five copies of cDOPA5GT integrated into its genome. In some embodiments, the cell comprises four copies of CYP76AD1, three copies of MjDOD, two copies of ATR2 and five copies of MjcDOPA5GT integrated into its genome. In some embodiments, the cell comprises four copies of CYP76AD1, three copies of MjDOD, two copies of ATR2 and five copies of cDOPA5GT integrated into its genome. In some embodiments, the cell comprises at least 5 copies of CYP76AD1, at least 2 copies of MjDOD, at least 4 copies of ATR2 and at least 3 copies of cDOPA5GT integrated into its genome. In some embodiments, the cell comprises 5 copies of CYP76AD1, 2 copies of MjDOD, 4 copies of ATR2 and 3 copies of cDOPA5GT integrated into its genome. In some embodiments, the cell comprises at least 5 copies of CYP76AD1, at least 3 copies of MjDOD, at least 4 copies of ATR2 and at least 3 copies of cDOPA5GT integrated into its genome. In some embodiments, the cell comprises 5 copies of CYP76AD1, 3 copies of MjDOD, 4 copies of ATR2 and 3 copies of cDOPA5GT integrated into its genome. In some embodiments, the cell further comprises at least one copy of ATR1 integrated into its genome. In some embodiments, the cell further comprises one copy of ATR1 integrated into its genome. In some embodiments, the cell comprises at least two copies of ATR2 and one copy of ATR1 integrated into its genome. In some embodiments, the cell comprises two copies of ATR2 and one copy of ATR1 integrated into its genome.

In some embodiments, the cell further comprises at least a copy of a Cytochrome reductase gene. In some embodiments, the cytochrome reductase is a cytochrome b5 reductase. In some embodiments, the cell further comprises at least a copy of a Cytochrome b5 reductase (Cyb5) gene. In some embodiments, the Cyb5 is exogenous to the cell. In some embodiments, the Cytochrome reductase is integrated into the genome of the cell. In some embodiments, the Cyb5 is integrated into the genome of the cell. In some embodiments, the Cyb5 gene is S. cerevisiae Cyb5. In some embodiments, the nucleic acid sequence encoding Cyb5 comprises SEQ ID NO: 13. In some embodiments, the nucleic acid sequence encoding Cyb5 consists of SEQ ID NO: 13. In some embodiments, the gene is without introns. In some embodiments, the gene is a cDNA of the gene. In some embodiments, the cytochrome reductase gene comprises SEQ ID NO: 13. In some embodiments, the cytochrome reductase gene consists of SEQ ID NO: 13. In some embodiments, the Cyb5 gene comprises a sequence with at least 70, 75, 80, 85, 90, 95, 97 or 99% identity to SEQ ID NO: 13 while retaining reductase activity. Each possibility represents a separate embodiment of the invention. In some embodiments, retaining activity is encoding a protein that retains activity. Other examples of Cyb5 genes include but are not limited to Yarrowia lipolytica cytochrorne b5 (YlCyb5), Pichia pastoris cytochrome b5 (PpCyb5), Beta vulgaris cytochrome b5 (BvCyb5),

In some embodiments, the amino acid sequence of Cyb5 comprises SEQ ID NO: 14. In some embodiments, the amino acid sequence of Cyb5 consists of SEQ ID NO: 14. In some embodiments, the Cyb5 gene comprises a nucleic acid sequence that encodes SEQ ID NO: 14. In some embodiments, the Cyb5 gene consists of a nucleic acid sequence that encodes SEQ ID NO: 14. In some embodiments, the Cyb5 gene comprises or consists of a nucleic acid sequence that encodes a protein with at least 70, 75, 80, 85, 90, 95, 97 or 99% identity to SEQ ID NO: 14. Each possibility represents a separate embodiment of the invention. In some embodiments, the protein retains reductase activity.

In some embodiments, the cell further comprises at least copy of a betanidin-5-O-glucosyltransferase gene. In some embodiments, a betanidin-5-O-glucosyltransferase is a 5-O-betanidin-glucosyltransferase. In some embodiments, the betanidin-5-O-glucosyltransferase is exogenous to the cell. In some embodiments, the betanidin-5-O-glucosyltransferase is integrated into the genome of the cell. In some embodiments, the betanidin-5-O-glucosyltransferase gene is a Dorotheanthus bellidiformis betanidin-5-O-glucosyltransferase (Db5GT). In some embodiments, the nucleic acid sequence encoding betanidin-5-O-glucosyltransferase comprises SEQ ID NO: 15. In some embodiments, the nucleic acid sequence encoding betanidin-5-O-glucosyltransferase consists of SEQ ID NO: 15. In some embodiments, the gene is without introns. In some embodiments, the gene is a cDNA of the gene. In some embodiments, the betanidin-5-O-glucosyltransferase gene comprises a sequence with at least 70, 75, 80, 85, 90, 95, 97 or 99% identity to SEQ ID NO: 15 while retaining glucosyltransferase activity. Each possibility represents a separate embodiment of the invention. In some embodiments, retaining activity is encoding a protein that retains activity.

In some embodiments, the amino acid sequence of the betanidin-5-O-glucosyltransferase comprises SEQ ID NO: 16. In some embodiments, the amino acid sequence of the betanidin-5-O-glucosyltransferase consists of SEQ ID NO: 16. In some embodiments, the betanidin-5-O-glucosyltransferase gene comprises a nucleic acid sequence that encodes SEQ ID NO: 16. In some embodiments, the betanidin-5-O-glucosyltransferase gene consists of a nucleic acid sequence that encodes SEQ ID NO: 16. In some embodiments, the betanidin-5-O-glucosyltransferase gene comprises or consists of a nucleic acid sequence that encodes a protein with at least 70, 75, 80, 85, 90, 95, 97 or 99% identity to SEQ ID NO: 16. Each possibility represents a separate embodiment of the invention. In some embodiments, the protein retains glucosyltransferase activity.

In some embodiments, the cell is further engineered to have increased production of UDP-glucose. In some embodiments, the cell further comprises at least one copy of a gene that increases production of UDP-glucose. In some embodiments, the cell comprises at least one copy of a gene that increases production of UDP-glucose integrated into its genome. In some embodiments, the gene is an exogenous gene to the cell. In some embodiments, the gene is a cDNA. In some embodiments, the gene is without introns. In some embodiments, the cell further comprises at least one mutation of an endogenous gene that results in increased production of UDP-glucose. In some embodiments, the mutation reduces gene function and thereby increases production of UDP-glucose. In some embodiments, the mutation is a knockout of the gene. As used herein, the term “knockout” refers to a mutation that results in no protein being produced. In some embodiments, no protein is no detectable protein. Methods of knocking out a gene are well known in the art and any such method may be used. For example, CRISPR gene editing may be used to knockout the gene. It may also be knocked-out by inserting coding or non-coding DNA that disrupts the open reading frame of the knocked-out gene. In some embodiments, the cell comprises knockout of at least one endogenous gene in the cell, wherein the knockout increases production of UDP-glucose in the cell.

In some embodiments, the gene that increases UDP-glucose is selected from UGP1, PGM2, URA6 and YNK1. In some embodiments, the gene that increases UDP-glucose is UGP1. In some embodiments, the UGP1 gene comprises SEQ ID NO: 17. In some embodiments, a UGP1 gene consists of SEQ ID NO: 17. In some embodiments, a UGP1 gene comprises at least 70, 75, 80, 85, 90, 92, 95, 97, 99 or 100% identity to SEQ ID NO: 17. Each possibility represents a separate embodiment of the invention. In some embodiments, an optimized UGP1 gene comprises SEQ ID NO: 31. In some embodiments, an optimized UGP1 gene consists of SEQ ID NO: 31. In some embodiments, SEQ ID NO: 31 encodes the S11A mutant protein that modulates phosphorylation. In some embodiments, a UGP1 gene comprises at least 70, 75, 80, 85, 90, 92, 95, 97, 99 or 100% identity to SEQ ID NO: 31. Each possibility represents a separate embodiment of the invention. In some embodiments, a UGP1 gene encodes a protein comprising the amino acid sequence of SEQ ID NO: 18. In some embodiments, a UGP1 gene encodes a protein consisting of the amino acid sequence of SEQ ID NO: 18. In some embodiments, a UGP1 gene encodes a protein comprising an amino acid sequence comprising at least 70, 75, 80, 85, 90, 92, 95, 97, 99 or 100% identity to SEQ ID NO: 18. Each possibility represents a separate embodiment of the invention. In some embodiments, a UGP1 gene encodes an optimized protein comprising the amino acid sequence of SEQ ID NO: 32. In some embodiments, a UGP1 gene encodes an optimized protein consisting of the amino acid sequence of SEQ ID NO: 32. In some embodiments, a UGP1 gene encodes an optimized protein comprising an amino acid sequence comprising at least 70, 75, 80, 85, 90, 92, 95, 97, 99 or 100% identity to SEQ ID NO: 32. Each possibility represents a separate embodiment of the invention. In some embodiments, the gene with less than 100% identity increases UDP-glucose.

In some embodiments, the gene that increases UDP-glucose is PGM2. In some embodiments, a PGM2 gene comprises SEQ ID NO: 19. In some embodiments, a PGM2 gene consists of SEQ ID NO: 19. In some embodiments, a PGM2 gene comprises at least 70, 75, 80, 85, 90, 92, 95, 97, 99 or 100% identity to SEQ ID NO: 19. Each possibility represents a separate embodiment of the invention. In some embodiments, an optimized PGM2 gene comprises SEQ ID NO: 33. In some embodiments, an optimized PGM2 gene consists of SEQ ID NO: 33. In some embodiments, an optimized PGM2 gene comprises at least 70, 75, 80, 85, 90, 92, 95, 97, 99 or 100% identity to SEQ ID NO: 33. Each possibility represents a separate embodiment of the invention. In some embodiments, optimized is codon optimized. In some embodiments, optimized is optimized to remove restriction sites. In some embodiments, optimized is optimized for expression in a cell. In some embodiments, a PGM2 gene encodes a protein comprising the amino acid sequence of SEQ ID NO: 20. In some embodiments, a PGM2 gene encodes a protein consisting of the amino acid sequence of SEQ ID NO: 20. In some embodiments, a PGM2 gene encodes a protein comprising an amino acid sequence comprising at least 70, 75, 80, 85, 90, 92, 95, 97, 99 or 100% identity to SEQ ID NO: 20. Each possibility represents a separate embodiment of the invention. In some embodiments, the gene with less than 100% identity increases UDP-glucose.

In some embodiments, the gene that increases UDP-glucose is URA6. In some embodiments, a URA6 gene comprises SEQ ID NO: 27. In some embodiments, a URA6 gene consists of SEQ ID NO: 27. In some embodiments, a URA6 gene comprises at least 70, 75, 80, 85, 90, 92, 95, 97, 99 or 100% identity to SEQ ID NO: 27. Each possibility represents a separate embodiment of the invention. In some embodiments, a URA6 gene encodes a protein comprising the amino acid sequence of SEQ ID NO: 28. In some embodiments, a URA6 gene encodes a protein consisting of the amino acid sequence of SEQ ID NO: 28. In some embodiments, a URA6 gene encodes a protein comprising an amino acid sequence comprising at least 70, 75, 80, 85, 90, 92, 95, 97, 99 or 100% identity to SEQ ID NO: 28. Each possibility represents a separate embodiment of the invention. In some embodiments, the gene with less than 100% identity increases UDP-glucose.

In some embodiments, the gene that increases UDP-glucose is YNK1. In some embodiments, a YNK1 gene comprises SEQ ID NO: 21. In some embodiments, a YNK1 gene consists of SEQ ID NO: 21. In some embodiments, a YNK1 gene comprises at least 70, 75, 80, 85, 90, 92, 95, 97, 99 or 100% identity to SEQ ID NO: 21. Each possibility represents a separate embodiment of the invention. In some embodiments, a YNK1 gene encodes a protein comprising the amino acid sequence of SEQ ID NO: 22. In some embodiments, a YNK1 gene encodes a protein consisting of the amino acid sequence of SEQ ID NO: 22. In some embodiments, a YNK1 gene encodes a protein comprising an amino acid sequence comprising at least 70, 75, 80, 85, 90, 92, 95, 97, 99 or 100% identity to SEQ ID NO: 22. Each possibility represents a separate embodiment of the invention. In some embodiments, the gene with less than 100% identity increases UDP-glucose.

In some embodiments, the endogenous gene is selected from exg1 and ynd1. In some embodiments, the endogenous gene whose mutation increases UDP-glucose is selected from exg1 and ynd1. In some embodiments, the endogenous gene is exg1. In some embodiments, the exg1 gene comprises SEQ ID NO: 23. In some embodiments, the exg1 gene consists of SEQ ID NO: 23. In some embodiments, the exg1 gene comprises a sequence with at least 70, 75, 80, 85, 90, 92, 95, 97, 99 or 100% identity to SEQ ID NO: 23. Each possibility represents a separate embodiment of the invention. In some embodiments, the exg1 gene encodes an mRNA comprising SEQ ID NO: 23. In some embodiments, the exg1 gene encodes an mRNA consisting of SEQ ID NO: 23. In some embodiments, the exg1 gene encodes an mRNA comprising at least 70, 75, 80, 85, 90, 92, 95, 97, 99 or 100% identity to SEQ ID NO: 23. Each possibility represents a separate embodiment of the invention. In some embodiments, the exg1 gene encodes a protein comprising the amino acid sequence provided in SEQ ID NO: 24. In some embodiments, the exg1 gene encodes a protein consisting of the amino acid sequence provided in SEQ ID NO: 24. In some embodiments, the exg1 gene encodes a protein comprising and amino acid sequence comprising at least 70, 75, 80, 85, 90, 92, 95, 97, 99 or 100% identity to SEQ ID NO: 24. Each possibility represents a separate embodiment of the invention. In some embodiments, mutation is knockout. In some embodiments, knockout is deletion.

In some embodiments, the endogenous gene is ynd1. In some embodiments, the ynd1 gene comprises SEQ ID NO: 25. In some embodiments, the ynd1 gene consists of SEQ ID NO: 25. In some embodiments, the ynd1 gene comprises a sequence with at least 70, 75, 80, 85, 90, 92, 95, 97, 99 or 100% identity to SEQ ID NO: 25. Each possibility represents a separate embodiment of the invention. In some embodiments, the ynd1 gene encodes an mRNA comprising SEQ ID NO: 25. In some embodiments, the ynd1 gene encodes an mRNA consisting of SEQ ID NO: 25. In some embodiments, the ynd1 gene encodes an mRNA comprising at least 70, 75, 80, 85, 90, 92, 95, 97, 99 or 100% identity to SEQ ID NO: 25. Each possibility represents a separate embodiment of the invention. In some embodiments, the ynd1 gene encodes a protein comprising the amino acid sequence provided in SEQ ID NO: 26. In some embodiments, the ynd1 gene encodes a protein consisting of the amino acid sequence provided in SEQ ID NO: 26. In some embodiments, the ynd1 gene encodes a protein comprising and amino acid sequence comprising at least 70, 75, 80, 85, 90, 92, 95, 97, 99 or 100% identity to SEQ ID NO: 26. Each possibility represents a separate embodiment of the invention. In some embodiments, mutation is knockout. In some embodiments, knockout is deletion.

In some embodiments, the cell comprises at least one copy of UGP1, PGM2, URA6 and YNK1 integrated into the genome of the cell. It will be understood that yeast have an endogenous copy of each of UGP1, PGM2, URA6 and YNK1 and the integration of an exogenous copy of these genes into the genome results in a cell with two copies each of UGP1, PGM2, URA6 and YNK1. In some embodiments, the cell comprises at least one copy of each of UGP1, PGM2, URA6 and YNK1 integrated into the genome of the cell. In some embodiments, the cell comprises at least one exogenous copy of UGP1, PGM2, URA6 and YNK1 integrated into the genome of the cell. In some embodiments, the cell comprises at least one exogenous copy of each of UGP1, PGM2, URA6 and YNK1 integrated into the genome of the cell. In some embodiments, the cell comprises knockout of exg1 and ydn1 in the cell. In some embodiments, the cell comprises knockout of both endogenous exg1 and endogenous ydn1. In some embodiments, the cell comprises at least one copy of UGP1, PGM2, URA6 and YNK1 integrated into the genome of the cell and knockout of endogenous exg1 and endogenous ydn1. In some embodiments, the cell comprises at least one copy of UGP1 integrated into the genome of the cell, at least one copy of PGM2 integrated into the genome of the cell, at least one copy of URA6 integrated into the genome of the cell, at least one copy of YNK1 integrated into the genome of the cell, knockout of endogenous exg1 and knockout of exogenous ydn1. In some embodiments, at least one copy is one copy.

In some embodiments, the cell is further engineered to have increased plasma membrane transport. In some embodiments, transport is export. In some embodiments, the cell is further engineered to have increased export out of the cell. In some embodiments, the cell further comprises at least one copy of a gene that increases export through the plasma membrane. In some embodiments, the cell further comprises at least one copy of a plasma membrane transport gene. In some embodiments, the cell further comprises at least one copy of a gene that increases export out of the cell. In some embodiments, the cell comprises at least one copy of a gene that increases export through the plasma membrane or export out of the cell integrated into its genome. In some embodiments, the cell further comprises at least one copy of a plasma membrane transport gene integrated into its genome. In some embodiments, the gene is an exogenous gene to the cell. In some embodiments, the gene is a cDNA. In some embodiments, the gene is without introns. In some embodiments, the gene is a yeast gene. In some embodiments, the plasma membrane transport gene is a yeast gene. In some embodiments, the cell comprises an exogenous second copy of an endogenous plasma membrane transport gene.

In some embodiments, the plasma membrane gene is QDR2. In some embodiments, the gene that increases transport through the plasma membrane or transport out of the cell is QDR2. In some embodiments, a QDR2 gene comprises SEQ ID NO: 34. In some embodiments, a QDR2 gene consists of SEQ ID NO: 34. In some embodiments, a QDR2 gene comprises at least 70, 75, 80, 85, 90, 92, 95, 97, 99 or 100% identity to SEQ ID NO: 34. Each possibility represents a separate embodiment of the invention. In some embodiments, a QDR2 gene encodes a protein comprising the amino acid sequence of SEQ ID NO: 35. In some embodiments, a QDR2 gene encodes a protein consisting of the amino acid sequence of SEQ ID NO: 35. In some embodiments, a QDR2 gene encodes a protein comprising an amino acid sequence comprising at least 70, 75, 80, 85, 90, 92, 95, 97, 99 or 100% identity to SEQ ID NO: 35. Each possibility represents a separate embodiment of the invention. In some embodiments, the gene with less than 100% identity increases transport through the plasma membrane, transport out of the cell or both. In some embodiments, the gene with less than 100% identity increases plasma membrane transport.

In some embodiments, the inserted genes are under the control of a strong promoter. In some embodiments, the genes are integrated into the genome with a promoter. In some embodiments, the promoter is operatively linked to the genes. The term “operably linked” is intended to mean that the nucleotide sequence of interest (i.e., the integrated genes) is linked to the regulatory element or elements in a manner that allows for expression of the nucleotide sequence. In some embodiments, the strong promoter is the GAL1/10 promoter. In some embodiments, all integrated genes are operatively linked to a GAL1/10 promoter. In some embodiments, the GAL1/10 promoter comprises or consists of the nucleotide sequence provided in SEQ ID NO: 44. In some embodiments, the GAL1/10 promoter is an optimized sequence.

In some embodiments, the inserted genes are linked to a terminator sequence. In some embodiments, the terminator is a strong terminator. It will be understood that being linked to a terminator sequence is intended to mean that the nucleotide sequence of interest (i.e., the integrated genes) proceeds the termination sequence or terminator in a manner that allows for termination of transcription at the end or just after the sequence of interest. Examples of terminators that may be used are the terminators of CYC1, ADH2, ADH1, and TEF2.

In some embodiments, the cell further comprises at least one mutation of a gene of the galactose metabolism pathway. In some embodiments, the cell comprises integrated genes under the control of the GAL1/10 promoter and at least one mutation of a gene of the galactose metabolism pathway. In some embodiments, the cell further comprises at least one mutation of a gene that increases galactose metabolism. In some embodiments, the cell further comprises at least one mutation of a gene required for galactose metabolism. In some embodiments, the mutation reduces gene function and thereby decreases galactose metabolism. In some embodiments, the mutation is a knockout of the gene. In some embodiments, no protein is produced. In some embodiments, no protein is no detectable protein. Methods of knocking out a gene are well known in the art and any such method may be used. For example, CRISPR gene editing may be used to knockout the gene. It may also be knocked-out by inserting coding or non-coding DNA that disrupts the open reading frame of the knocked-out gene. In some embodiments, the cell comprises knockout of at least one endogenous gene in the cell, wherein the knockout decreases galactose metabolism. In some embodiments, the cell comprises knockout of at least one endogenous galactose metabolism gene.

In some embodiments, the endogenous gene is selected from gal80, gal1 and gal10. In some embodiments, the endogenous gene whose mutation decreases galactose metabolism is selected from gal80, gal1 and gal10. In some embodiments, the gene of the galactose metabolism pathway is selected from gal80, gal1 and gal10. In some embodiments, the cell comprises knockdown of at least two of endogenous gal80, endogenous gal1 and endogenous gal10. In some embodiments, the cell comprises knockdown of endogenous gal80, endogenous gal1 and endogenous gal10.

In some embodiments, the endogenous gene is gal80. In some embodiments, the gal80 gene comprises SEQ ID NO: 36. In some embodiments, the gal80 gene consists of SEQ ID NO: 36. In some embodiments, the gal80 gene comprises a sequence with at least 70, 75, 80, 85, 90, 92, 95, 97, 99 or 100% identity to SEQ ID NO: 36. Each possibility represents a separate embodiment of the invention. In some embodiments, the gal80 gene encodes an mRNA comprising SEQ ID NO: 36. In some embodiments, the gal80 gene encodes an mRNA consisting of SEQ ID NO: 36. In some embodiments, the gal80 gene encodes an mRNA comprising at least 70, 75, 80, 85, 90, 92, 95, 97, 99 or 100% identity to SEQ ID NO: 36. Each possibility represents a separate embodiment of the invention. In some embodiments, the gal80 gene encodes a protein comprising the amino acid sequence provided in SEQ ID NO: 37. In some embodiments, the gal80 gene encodes a protein consisting of the amino acid sequence provided in SEQ ID NO: 37. In some embodiments, the gal80 gene encodes a protein comprising and amino acid sequence comprising at least 70, 75, 80, 85, 90, 92, 95, 97, 99 or 100% identity to SEQ ID NO: 37. Each possibility represents a separate embodiment of the invention. In some embodiments, mutation is knockout. In some embodiments, knockout is deletion.

In some embodiments, the endogenous gene is gal1. In some embodiments, the gal1 gene comprises SEQ ID NO: 38. In some embodiments, the gal1 gene consists of SEQ ID NO: 38. In some embodiments, the gal1 gene comprises a sequence with at least 70, 75, 80, 85, 90, 92, 95, 97, 99 or 100% identity to SEQ ID NO: 38. Each possibility represents a separate embodiment of the invention. In some embodiments, the gal1 gene encodes an mRNA comprising SEQ ID NO: 38. In some embodiments, the gal1 gene encodes an mRNA consisting of SEQ ID NO: 38. In some embodiments, the gal1 gene encodes an mRNA comprising at least 70, 75, 80, 85, 90, 92, 95, 97, 99 or 100% identity to SEQ ID NO: 38. Each possibility represents a separate embodiment of the invention. In some embodiments, the gal1 gene encodes a protein comprising the amino acid sequence provided in SEQ ID NO: 39. In some embodiments, the gal1 gene encodes a protein consisting of the amino acid sequence provided in SEQ ID NO: 39. In some embodiments, the gal1 gene encodes a protein comprising and amino acid sequence comprising at least 70, 75, 80, 85, 90, 92, 95, 97, 99 or 100% identity to SEQ ID NO: 39. Each possibility represents a separate embodiment of the invention. In some embodiments, mutation is knockout. In some embodiments, knockout is deletion.

In some embodiments, the endogenous gene is gal10. In some embodiments, the gal10 gene comprises SEQ ID NO: 40. In some embodiments, the gal10 gene consists of SEQ ID NO: 40. In some embodiments, the gal10 gene comprises a sequence with at least 70, 75, 80, 85, 90, 92, 95, 97, 99 or 100% identity to SEQ ID NO: 40. Each possibility represents a separate embodiment of the invention. In some embodiments, the gal10 gene encodes an mRNA comprising SEQ ID NO: 40. In some embodiments, the gal10 gene encodes an mRNA consisting of SEQ ID NO: 40. In some embodiments, the gal10 gene encodes an mRNA comprising at least 70, 75, 80, 85, 90, 92, 95, 97, 99 or 100% identity to SEQ ID NO: 40. Each possibility represents a separate embodiment of the invention. In some embodiments, the gal10 gene encodes a protein comprising the amino acid sequence provided in SEQ ID NO: 41. In some embodiments, the gal10 gene encodes a protein consisting of the amino acid sequence provided in SEQ ID NO: 41. In some embodiments, the gal10 gene encodes a protein comprising and amino acid sequence comprising at least 70, 75, 80, 85, 90, 92, 95, 97, 99 or 100% identity to SEQ ID NO: 41. Each possibility represents a separate embodiment of the invention. In some embodiments, mutation is knockout. In some embodiments, knockout is deletion.

By another aspect, there is provided a pigment composition produced by the cells.

In some embodiments, the pigment composition is secreted by the cells. In some embodiments, the pigment composition is from lysing the cells. In some embodiments, the pigment composition is produced without lysing the cells. In some embodiments, the pigment composition is produced by processing the media in which the cells are grown.

By another aspect, there is provided a method of producing a pigment composition, the method comprising culturing a cell of the invention in a culture medium and collecting said culture medium, thereby producing a pigment composition.

In some embodiments, the pigment composition is a composition of the invention. In some embodiments, the culturing is for a time sufficient for the cells to secrete betalains. In some embodiments, the culturing is in the absence of L-DOPA supplementation. In some embodiments, the method further comprises downstream processing of the medium. In some embodiments, the downstream processing comprises isolating the betalains from the media. In some embodiments, the processing comprises centrifugation. In some embodiments, the processing comprises filtration. In some embodiments, the filtration comprises microfiltration. In some embodiments, the filtration comprises ultrafiltration. In some embodiments, the filtration comprises nanofiltration. In some embodiments, the filtration comprises reverse osmosis. In some embodiments, the downstream processing comprises evaporation. In some embodiments, the downstream processing comprises evaporation under vacuum conditions. In some embodiments, the downstream processing comprises dehydration. In some embodiments, the downstream processing comprises crystallization. In some embodiments, the downstream processing comprises drying. In some embodiments, the downstream processing comprises freeze drying. In some embodiments, the downstream processing comprises spray drying. In some embodiments, the downstream processing comprises fluidizing. In some embodiments, the downstream processing comprises filtration. In some embodiments, the filtration is sterile filtration. In some embodiments, the culturing is for a period of time sufficient to produce pigment. The pigment is a pigment such as is described hereinabove.

In some embodiments, the composition is a powder. In some embodiments, the powder is powdered betalains. Betalains do not occur as a powder in nature and thus a pigment powder is inherently non-naturally occurring. In some embodiments, the powder comprises particles. In some embodiments, the particles are particles of betalains. In some embodiments, the particles are particles of betalains and additive. In some embodiments, the average particle size in the powder is 0.1-500, 0.1-450, 0.1-400, 0.1-350, 0.1-300, 0.1-250, 0.1-200, 0.1-150, 0.1-100, 0.1-95, 0.1-90, 0.1-85, 0.1-80, 0.1-75, 0.1-70, 0.1-70, 0.1-65, 0.1-60, 0.1-55, 0.1-50, 0.1-45, 0.1-40, 0.1-35, 0.1-34.5, 0.1-34, 0.1-33.5, 0.1-33, 0.1-32.5, 0.1-32, 0.1-31.5, 0.1-31, 0.1-30.5, 0.1-30, 0.1-29.5, 0.1-29, 0.1-28.5, 0.1-28, 0.1-27.5, 0.1-27, 0.1-26.5, 0.1-26, 0.1-25.5, 0.1-25, 0.1-24.5, 0.1-24, 0.1-23.5, 0.1-23, 0.1-22.5, 0.1-22, 0.1-21.5, 0.1-21, 0.1-20.5, 0.1-20, 0.1-19.5, 0.1-19, 0.1-18.5, 0.1-18, 0.1-17.5, 0.1-17, 0.1-16.5, 0.1-16, 0.1-15.5, 0.1-15, 0.1-14.5, 0.1-14, 0.1-13.5, 0.1-13, 0.1-12.5, 0.1-12, 0.1-11.5, 0.1-11, 0.1-10.5, 0.1-10, 0.1-9.5, 0.1-9, 0.1-8.5, 0.1-8, 0.1-7.5, 0.1-7, 0.1-6.5, 0.1-6, 0.1-5.5, 0.1-5, 0.1-4.5, 0.1-4, 0.1-3.5, 0.1-3, 0.1-2.5, 0.1-2, 0.1-1.5, 0.1-1, 0.1-0.9, 0.1-0.8, 0.1-0.7, 0.1-0.6, 0.1-0.5, 0.1-0.4, 0.1-0.3, 0.1-0.2, 0.5-500, 0.5-450, 0.5-400, 0.5-350, 0.5-300, 0.5-250, 0.5-200, 0.5-150, 0.5-100, 0.5-95, 0.5-90, 0.5-85, 0.5-80, 0.5-75, 0.5-70, 0.5-70, 0.5-65, 0.5-60, 0.5-55, 0.5-50, 0.5-45, 0.5-40, 0.5-35, 0.5-34.5, 0.5-34, 0.5-33.5, 0.5-33, 0.5-32.5, 0.5-32, 0.5-31.5, 0.5-31, 0.5-30.5, 0.5-30, 0.5-29.5, 0.5-29, 0.5-28.5, 0.5-28, 0.5-27.5, 0.5-27, 0.5-26.5, 0.5-26, 0.5-25.5, 0.5-25, 0.5-24.5, 0.5-24, 0.5-23.5, 0.5-23, 0.5-22.5, 0.5-22, 0.5-21.5, 0.5-21, 0.5-20.5, 0.5-20, 0.5-19.5, 0.5-19, 0.5-18.5, 0.5-18, 0.5-17.5, 0.5-17, 0.5-16.5, 0.5-16, 0.5-15.5, 0.5-15, 0.5-14.5, 0.5-14, 0.5-13.5, 0.5-13, 0.5-12.5, 0.5-12, 0.5-11.5, 0.5-11, 0.5-10.5, 0.5-10, 0.5-9.5, 0.5-9, 0.5-8.5, 0.5-8, 0.5-7.5, 0.5-7, 0.5-6.5, 0.5-6, 0.5-5.5, 0.5-5, 0.5-4.5, 0.5-4, 0.5-3.5, 0.5-3, 0.5-2.5, 0.5-2, 0.5-1.5, 0.5-1, 0.5-0.9, 0.5-0.8, 0.5-0.7, 0.5-0.6, 1-500, 1-450, 1-400, 1-350, 1-300, 1-250, 1-200, 1-150, 1-100, 1-95, 1-90, 1-85, 1-80, 1-75, 1-70, 1-70, 1-65, 1-60, 1-55, 1-50, 1-45, 1-40, 1-35, 1-34.5, 1-34, 1-33.5, 1-33, 1-32.5, 1-32, 1-31.5, 1-31, 1-30.5, 1-30, 1-29.5, 1-29, 1-28.5, 1-28, 1-27.5, 1-27, 1-26.5, 1-26, 1-25.5, 1-25, 1-24.5, 1-24, 1-23.5, 1-23, 1-22.5, 1-22, 1-21.5, 1-21, 1-20.5, 1-20, 1-19.5, 1-19, 1-18.5, 1-18, 1-17.5, 1-17, 1-16.5, 1-16, 1-15.5, 1-15, 1-14.5, 1-14, 1-13.5, 1-13, 1-12.5, 1-12, 1-11.5, 1-11, 1-10.5, 1-10, 1-9.5, 1-9, 1-8.5, 1-8, 1-7.5, 1-7, 1-6.5, 1-6, 1-5.5, 1-5, 1-4.5, 1-4, 1-3.5, 1-3, 1-2.5, 1-2, 1-1.5, 1.5-500, 1.5-450, 1.5-400, 1.5-350, 1.5-300, 1.5-250, 1.5-200, 1.5-150, 1.5-100, 1.5-95, 1.5-90, 1.5-85, 1.5-80, 1.5-75, 1.5-70, 1.5-70, 1.5-65, 1.5-60, 1.5-55, 1.5-50, 1.5-45, 1.5-40, 1.5-35, 1.5-34.5, 1.5-34, 1.5-33.5, 1.5-33, 1.5-32.5, 1.5-32, 1.5-31.5, 1.5-31, 1.5-30.5, 1.5-30, 1.5-29.5, 1.5-29, 1.5-28.5, 1.5-28, 1.5-27.5, 1.5-27, 1.5-26.5, 1.5-26, 1.5-25.5, 1.5-25, 1.5-24.5, 1.5-24, 1.5-23.5, 1.5-23, 1.5-22.5, 1.5-22, 1.5-21.5, 1.5-21, 1.5-20.5, 1.5-20, 1.5-19.5, 1.5-19, 1.5-18.5, 1.5-18, 1.5-17.5, 1.5-17, 1.5-16.5, 1.5-16, 1.5-15.5, 1.5-15, 1.5-14.5, 1.5-14, 1.5-13.5, 1.5-13, 1.5-12.5, 1.5-12, 1.5-11.5, 1.5-11, 1.5-10.5, 1.5-10, 1.5-9.5, 1.5-9, 1.5-8.5, 1.5-8, 1.5-7.5, 1.5-7, 1.5-6.5, 1.5-6, 1.5-5.5, 1.5-5, 1.5-4.5, 1.5-4, 1.5-3.5, 1.5-3, 1.5-2.5, 1.5-2, 2-500, 2-450, 2-400, 2-350, 2-300, 2-250, 2-200, 2-150, 2-100, 2-95, 2-90, 2-85, 2-80, 2-75, 2-70, 2-70, 2-65, 2-60, 2-55, 2-50, 2-45, 2-40, 2-35, 2-34.5, 2-34, 2-33.5, 2-33, 2-32.5, 2-32, 2-31.5, 2-31, 2-30.5, 2-30, 2-29.5, 2-29, 2-28.5, 2-28, 2-27.5, 2-27, 2-26.5, 2-26, 2-25.5, 2-25, 2-24.5, 2-24, 2-23.5, 2-23, 2-22.5, 2-22, 2-21.5, 2-21, 2-20.5, 2-20, 2-19.5, 2-19, 2-18.5, 2-18, 2-17.5, 2-17, 2-16.5, 2-16, 2-15.5, 2-15, 2-14.5, 2-14, 2-13.5, 2-13, 2-12.5, 2-12, 2-11.5, 2-11, 2-10.5, 2-10, 2-9.5, 2-9, 2-8.5, 2-8, 2-7.5, 2-7, 2-6.5, 2-6, 2-5.5, 2-5, 2-4.5, 2-4, 2-3.5, 2-3, 2-2.5, 2.5-500, 2.5-450, 2.5-400, 2.5-350, 2.5-300, 2.5-250, 2.5-200, 2.5-150, 2.5-100, 2.5-95, 2.5-90, 2.5-85, 2.5-80, 2.5-75, 2.5-70, 2.5-70, 2.5-65, 2.5-60, 2.5-55, 2.5-50, 2.5-45, 2.5-40, 2.5-35, 2.5-34.5, 2.5-34, 2.5-33.5, 2.5-33, 2.5-32.5, 2.5-32, 2.5-31.5, 2.5-31, 2.5-30.5, 2.5-30, 2.5-29.5, 2.5-29, 2.5-28.5, 2.5-28, 2.5-27.5, 2.5-27, 2.5-26.5, 2.5-26, 2.5-25.5, 2.5-25, 2.5-24.5, 2.5-24, 2.5-23.5, 2.5-23, 2.5-22.5, 2.5-22, 2.5-21.5, 2.5-21, 2.5-20.5, 2.5-20, 2.5-19.5, 2.5-19, 2.5-18.5, 2.5-18, 2.5-17.5, 2.5-17, 2.5-16.5, 2.5-16, 2.5-15.5, 2.5-15, 2.5-14.5, 2.5-14, 2.5-13.5, 2.5-13, 2.5-12.5, 2.5-12, 2.5-11.5, 2.5-11, 2.5-10.5, 2.5-10, 2.5-9.5, 2.5-9, 2.5-8.5, 2.5-8, 2.5-7.5, 2.5-7, 2.5-6.5, 2.5-6, 2.5-5.5, 2.5-5, 2.5-4.5, 2.5-4, 2.5-3.5, 2.5-3, 3-500, 3-450, 3-400, 3-350, 3-300, 3-250, 3-200, 3-150, 3-100, 3-95, 3-90, 3-85, 3-80, 3-75, 3-70, 3-70, 3-65, 3-60, 3-55, 3-50, 3-45, 3-40, 3-35, 3-34.5, 3-34, 3-33.5, 3-33, 3-32.5, 3-32, 3-31.5, 3-31, 3-30.5, 3-30, 3-29.5, 3-29, 3-28.5, 3-28, 3-27.5, 3-27, 3-26.5, 3-26, 3-25.5, 3-25, 3-24.5, 3-24, 3-23.5, 3-23, 3-22.5, 3-22, 3-21.5, 3-21, 3-20.5, 3-20, 3-19.5, 3-19, 3-18.5, 3-18, 3-17.5, 3-17, 3-16.5, 3-16, 3-15.5, 3-15, 3-14.5, 3-14, 3-13.5, 3-13, 3-12.5, 3-12, 3-11.5, 3-11, 3-10.5, 3-10, 3-9.5, 3-9, 3-8.5, 3-8, 3-7.5, 3-7, 3-6.5, 3-6, 3-5.5, 3-5, 3-4.5, 3-4, 3-3.5, 3.5-500, 3.5-450, 3.5-400, 3.5-350, 3.5-300, 3.5-250, 3.5-200, 3.5-150, 3.5-100, 3.5-95, 3.5-90, 3.5-85, 3.5-80, 3.5-75, 3.5-70, 3.5-70, 3.5-65, 3.5-60, 3.5-55, 3.5-50, 3.5-45, 3.5-40, 3.5-35, 3.5-34.5, 3.5-34, 3.5-33.5, 3.5-33, 3.5-32.5, 3.5-32, 3.5-31.5, 3.5-31, 3.5-30.5, 3.5-30, 3.5-29.5, 3.5-29, 3.5-28.5, 3.5-28, 3.5-27.5, 3.5-27, 3.5-26.5, 3.5-26, 3.5-25.5, 3.5-25, 3.5-24.5, 3.5-24, 3.5-23.5, 3.5-23, 3.5-22.5, 3.5-22, 3.5-21.5, 3.5-21, 3.5-20.5, 3.5-20, 3.5-19.5, 3.5-19, 3.5-18.5, 3.5-18, 3.5-17.5, 3.5-17, 3.5-16.5, 3.5-16, 3.5-15.5, 3.5-15, 3.5-14.5, 3.5-14, 3.5-13.5, 3.5-13, 3.5-12.5, 3.5-12, 3.5-11.5, 3.5-11, 3.5-10.5, 3.5-10, 3.5-9.5, 3.5-9, 3.5-8.5, 3.5-8, 3.5-7.5, 3.5-7, 3.5-6.5, 3.5-6, 3.5-5.5, 3.5-5, 3.5-4.5, 3.5-4, 4-500, 4-450, 4-400, 4-350, 4-300, 4-250, 4-200, 4-150, 4-100, 4-95, 4-90, 4-85, 4-80, 4-75, 4-70, 4-70, 4-65, 4-60, 4-55, 4-50, 4-45, 4-40, 4-35, 4-34.5, 4-34, 4-33.5, 4-33, 4-32.5, 4-32, 4-31.5, 4-31, 4-30.5, 4-30, 4-29.5, 4-29, 4-28.5, 4-28, 4-27.5, 4-27, 4-26.5, 4-26, 4-25.5, 4-25, 4-24.5, 4-24, 4-23.5, 4-23, 4-22.5, 4-22, 4-21.5, 4-21, 4-20.5, 4-20, 4-19.5, 4-19, 4-18.5, 4-18, 4-17.5, 4-17, 4-16.5, 4-16, 4-15.5, 4-15, 4-14.5, 4-14, 4-13.5, 4-13, 4-12.5, 4-12, 4-11.5, 4-11, 4-10.5, 4-10, 4-9.5, 4-9, 4-8.5, 4-8, 4-7.5, 4-7, 4-6.5, 4-6, 4-5.5, 4-5, 4-4.5, 4.5-500, 4.5-450, 4.5-400, 4.5-350, 4.5-300, 4.5-250, 4.5-200, 4.5-150, 4.5-100, 4.5-95, 4.5-90, 4.5-85, 4.5-80, 4.5-75, 4.5-70, 4.5-70, 4.5-65, 4.5-60, 4.5-55, 4.5-50, 4.5-45, 4.5-40, 4.5-35, 4.5-34.5, 4.5-34, 4.5-33.5, 4.5-33, 4.5-32.5, 4.5-32, 4.5-31.5, 4.5-31, 4.5-30.5, 4.5-30, 4.5-29.5, 4.5-29, 4.5-28.5, 4.5-28, 4.5-27.5, 4.5-27, 4.5-26.5, 4.5-26, 4.5-25.5, 4.5-25, 4.5-24.5, 4.5-24, 4.5-23.5, 4.5-23, 4.5-22.5, 4.5-22, 4.5-21.5, 4.5-21, 4.5-20.5, 4.5-20, 4.5-19.5, 4.5-19, 4.5-18.5, 4.5-18, 4.5-17.5, 4.5-17, 4.5-16.5, 4.5-16, 4.5-15.5, 4.5-15, 4.5-14.5, 4.5-14, 4.5-13.5, 4.5-13, 4.5-12.5, 4.5-12, 4.5-11.5, 4.5-11, 4.5-10.5, 4.5-10, 4.5-9.5, 4.5-9, 4.5-8.5, 4.5-8, 4.5-7.5, 4.5-7, 4.5-6.5, 4.5-6, 4.5-5.5, 4.5-5, 5-500, 5-450, 5-400, 5-350, 5-300, 5-250, 5-200, 5-150, 5-100, 5-95, 5-90, 5-85, 5-80, 5-75, 5-70, 5-70, 5-65, 5-60, 5-55, 5-50, 5-45, 5-40, 5-35, 5-34.5, 5-34, 5-33.5, 5-33, 5-32.5, 5-32, 5-31.5, 5-31, 5-30.5, 5-30, 5-29.5, 5-29, 5-28.5, 5-28, 5-27.5, 5-27, 5-26.5, 5-26, 5-25.5, 5-25, 5-24.5, 5-24, 5-23.5, 5-23, 5-22.5, 5-22, 5-21.5, 5-21, 5-20.5, 5-20, 5-19.5, 5-19, 5-18.5, 5-18, 5-17.5, 5-17, 5-16.5, 5-16, 5-15.5, 5-15, 5-14.5, 5-14, 5-13.5, 5-13, 5-12.5, 5-12, 5-11.5, 5-11, 5-10.5, 5-10, 5-9.5, 5-9, 5-8.5, 5-8, 5-7.5, 5-7, 5-6.5, 5-6, 5-5.5, 5.5-500, 5.5-450, 5.5-400, 5.5-350, 5.5-300, 5.5-250, 5.5-200, 5.5-150, 5.5-100, 5.5-95, 5.5-90, 5.5-85, 5.5-80, 5.5-75, 5.5-70, 5.5-70, 5.5-65, 5.5-60, 5.5-55, 5.5-50, 5.5-45, 5.5-40, 5.5-35, 5.5-34.5, 5.5-34, 5.5-33.5, 5.5-33, 5.5-32.5, 5.5-32, 5.5-31.5, 5.5-31, 5.5-30.5, 5.5-30, 5.5-29.5, 5.5-29, 5.5-28.5, 5.5-28, 5.5-27.5, 5.5-27, 5.5-26.5, 5.5-26, 5.5-25.5, 5.5-25, 5.5-24.5, 5.5-24, 5.5-23.5, 5.5-23, 5.5-22.5, 5.5-22, 5.5-21.5, 5.5-21, 5.5-20.5, 5.5-20, 5.5-19.5, 5.5-19, 5.5-18.5, 5.5-18, 5.5-17.5, 5.5-17, 5.5-16.5, 5.5-16, 5.5-15.5, 5.5-15, 5.5-14.5, 5.5-14, 5.5-13.5, 5.5-13, 5.5-12.5, 5.5-12, 5.5-11.5, 5.5-11, 5.5-10.5, 5.5-10, 5.5-9.5, 5.5-9, 5.5-8.5, 5.5-8, 5.5-7.5, 5.5-7, 5.5-6.5, 5.5-6, 6-500, 6-450, 6-400, 6-350, 6-300, 6-250, 6-200, 6-150, 6-100, 6-95, 6-90, 6-85, 6-80, 6-75, 6-70, 6-70, 6-65, 6-60, 6-55, 6-50, 6-45, 6-40, 6-35, 6-34.5, 6-34, 6-33.5, 6-33, 6-32.5, 6-32, 6-31.5, 6-31, 6-30.5, 6-30, 6-29.5, 6-29, 6-28.5, 6-28, 6-27.5, 6-27, 6-26.5, 6-26, 6-25.5, 6-25, 6-24.5, 6-24, 6-23.5, 6-23, 6-22.5, 6-22, 6-21.5, 6-21, 6-20.5, 6-20, 6-19.5, 6-19, 6-18.5, 6-18, 6-17.5, 6-17, 6-16.5, 6-16, 6-15.5, 6-15, 6-14.5, 6-14, 6-13.5, 6-13, 6-12.5, 6-12, 6-11.5, 6-11, 6-10.5, 6-10, 6-9.5, 6-9, 6-8.5, 6-8, 6-7.5, 6-7, 6-6.5, 6.5-500, 6.5-450, 6.5-400, 6.5-350, 6.5-300, 6.5-250, 6.5-200, 6.5-150, 6.5-100, 6.5-95, 6.5-90, 6.5-85, 6.5-80, 6.5-75, 6.5-70, 6.5-70, 6.5-65, 6.5-60, 6.5-55, 6.5-50, 6.5-45, 6.5-40, 6.5-35, 6.5-34.5, 6.5-34, 6.5-33.5, 6.5-33, 6.5-32.5, 6.5-32, 6.5-31.5, 6.5-31, 6.5-30.5, 6.5-30, 6.5-29.5, 6.5-29, 6.5-28.5, 6.5-28, 6.5-27.5, 6.5-27, 6.5-26.5, 6.5-26, 6.5-25.5, 6.5-25, 6.5-24.5, 6.5-24, 6.5-23.5, 6.5-23, 6.5-22.5, 6.5-22, 6.5-21.5, 6.5-21, 6.5-20.5, 6.5-20, 6.5-19.5, 6.5-19, 6.5-18.5, 6.5-18, 6.5-17.5, 6.5-17, 6.5-16.5, 6.5-16, 6.5-15.5, 6.5-15, 6.5-14.5, 6.5-14, 6.5-13.5, 6.5-13, 6.5-12.5, 6.5-12, 6.5-11.5, 6.5-11, 6.5-10.5, 6.5-10, 6.5-9.5, 6.5-9, 6.5-8.5, 6.5-8, 6.5-7.5, 6.5-7, 7-500, 7-450, 7-400, 7-350, 7-300, 7-250, 7-200, 7-150, 7-100, 7-95, 7-90, 7-85, 7-80, 7-75, 7-70, 7-70, 7-65, 7-60, 7-55, 7-50, 7-45, 7-40, 7-35, 7-34.5, 7-34, 7-33.5, 7-33, 7-32.5, 7-32, 7-31.5, 7-31, 7-30.5, 7-30, 7-29.5, 7-29, 7-28.5, 7-28, 7-27.5, 7-27, 7-26.5, 7-26, 7-25.5, 7-25, 7-24.5, 7-24, 7-23.5, 7-23, 7-22.5, 7-22, 7-21.5, 7-21, 7-20.5, 7-20, 7-19.5, 7-19, 7-18.5, 7-18, 7-17.5, 7-17, 7-16.5, 7-16, 7-15.5, 7-15, 7-14.5, 7-14, 7-13.5, 7-13, 7-12.5, 7-12, 7-11.5, 7-11, 7-10.5, 7-10, 7-9.5, 7-9, 7-8.5, 7-8, 7-7.5, 7.5-500, 7.5-450, 7.5-400, 7.5-350, 7.5-300, 7.5-250, 7.5-200, 7.5-150, 7.5-100, 7.5-95, 7.5-90, 7.5-85, 7.5-80, 7.5-75, 7.5-70, 7.5-70, 7.5-65, 7.5-60, 7.5-55, 7.5-50, 7.5-45, 7.5-40, 7.5-35, 7.5-34.5, 7.5-34, 7.5-33.5, 7.5-33, 7.5-32.5, 7.5-32, 7.5-31.5, 7.5-31, 7.5-30.5, 7.5-30, 7.5-29.5, 7.5-29, 7.5-28.5, 7.5-28, 7.5-27.5, 7.5-27, 7.5-26.5, 7.5-26, 7.5-25.5, 7.5-25, 7.5-24.5, 7.5-24, 7.5-23.5, 7.5-23, 7.5-22.5, 7.5-22, 7.5-21.5, 7.5-21, 7.5-20.5, 7.5-20, 7.5-19.5, 7.5-19, 7.5-18.5, 7.5-18, 7.5-17.5, 7.5-17, 7.5-16.5, 7.5-16, 7.5-15.5, 7.5-15, 7.5-14.5, 7.5-14, 7.5-13.5, 7.5-13, 7.5-12.5, 7.5-12, 7.5-11.5, 7.5-11, 7.5-10.5, 7.5-10, 7.5-9.5, 7.5-9, 7.5-8.5, 7.5-8, 8-500, 8-450, 8-400, 8-350, 8-300, 8-250, 8-200, 8-150, 8-100, 8-95, 8-90, 8-85, 8-80, 8-75, 8-70, 8-70, 8-65, 8-60, 8-55, 8-50, 8-45, 8-40, 8-35, 8-34.5, 8-34, 8-33.5, 8-33, 8-32.5, 8-32, 8-31.5, 8-31, 8-30.5, 8-30, 8-29.5, 8-29, 8-28.5, 8-28, 8-27.5, 8-27, 8-26.5, 8-26, 8-25.5, 8-25, 8-24.5, 8-24, 8-23.5, 8-23, 8-22.5, 8-22, 8-21.5, 8-21, 8-20.5, 8-20, 8-19.5, 8-19, 8-18.5, 8-18, 8-17.5, 8-17, 8-16.5, 8-16, 8-15.5, 8-15, 8-14.5, 8-14, 8-13.5, 8-13, 8-12.5, 8-12, 8-11.5, 8-11, 8-10.5, 8-10, 8-9.5, 8-9, 8-8.5, 8.5-500, 8.5-450, 8.5-400, 8.5-350, 8.5-300, 8.5-250, 8.5-200, 8.5-150, 8.5-100, 8.5-95, 8.5-90, 8.5-85, 8.5-80, 8.5-75, 8.5-70, 8.5-70, 8.5-65, 8.5-60, 8.5-55, 8.5-50, 8.5-45, 8.5-40, 8.5-35, 8.5-34.5, 8.5-34, 8.5-33.5, 8.5-33, 8.5-32.5, 8.5-32, 8.5-31.5, 8.5-31, 8.5-30.5, 8.5-30, 8.5-29.5, 8.5-29, 8.5-28.5, 8.5-28, 8.5-27.5, 8.5-27, 8.5-26.5, 8.5-26, 8.5-25.5, 8.5-25, 8.5-24.5, 8.5-24, 8.5-23.5, 8.5-23, 8.5-22.5, 8.5-22, 8.5-21.5, 8.5-21, 8.5-20.5, 8.5-20, 8.5-19.5, 8.5-19, 8.5-18.5, 8.5-18, 8.5-17.5, 8.5-17, 8.5-16.5, 8.5-16, 8.5-15.5, 8.5-15, 8.5-14.5, 8.5-14, 8.5-13.5, 8.5-13, 8.5-12.5, 8.5-12, 8.5-11.5, 8.5-11, 8.5-10.5, 8.5-10, 8.5-9.5, 8.5-9, 9-500, 9-450, 9-400, 9-350, 9-300, 9-250, 9-200, 9-150, 9-100, 9-95, 9-90, 9-85, 9-80, 9-75, 9-70, 9-70, 9-65, 9-60, 9-55, 9-50, 9-45, 9-40, 9-35, 9-34.5, 9-34, 9-33.5, 9-33, 9-32.5, 9-32, 9-31.5, 9-31, 9-30.5, 9-30, 9-29.5, 9-29, 9-28.5, 9-28, 9-27.5, 9-27, 9-26.5, 9-26, 9-25.5, 9-25, 9-24.5, 9-24, 9-23.5, 9-23, 9-22.5, 9-22, 9-21.5, 9-21, 9-20.5, 9-20, 9-19.5, 9-19, 9-18.5, 9-18, 9-17.5, 9-17, 9-16.5, 9-16, 9-15.5, 9-15, 9-14.5, 9-14, 9-13.5, 9-13, 9-12.5, 9-12, 9-11.5, 9-11, 9-10.5, 9-10, 9-9.5, 9.5-500, 9.5-450, 9.5-400, 9.5-350, 9.5-300, 9.5-250, 9.5-200, 9.5-150, 9.5-100, 9.5-95, 9.5-90, 9.5-85, 9.5-80, 9.5-75, 9.5-70, 9.5-70, 9.5-65, 9.5-60, 9.5-55, 9.5-50, 9.5-45, 9.5-40, 9.5-35, 9.5-34.5, 9.5-34, 9.5-33.5, 9.5-33, 9.5-32.5, 9.5-32, 9.5-31.5, 9.5-31, 9.5-30.5, 9.5-30, 9.5-29.5, 9.5-29, 9.5-28.5, 9.5-28, 9.5-27.5, 9.5-27, 9.5-26.5, 9.5-26, 9.5-25.5, 9.5-25, 9.5-24.5, 9.5-24, 9.5-23.5, 9.5-23, 9.5-22.5, 9.5-22, 9.5-21.5, 9.5-21, 9.5-20.5, 9.5-20, 9.5-19.5, 9.5-19, 9.5-18.5, 9.5-18, 9.5-17.5, 9.5-17, 9.5-16.5, 9.5-16, 9.5-15.5, 9.5-15, 9.5-14.5, 9.5-14, 9.5-13.5, 9.5-13, 9.5-12.5, 9.5-12, 9.5-11.5, 9.5-11, 9.5-10.5, 9.5-10, 10-500, 10-450, 10-400, 10-350, 10-300, 10-250, 10-200, 10-150, 10-100, 10-95, 10-90, 10-85, 10-80, 10-75, 10-70, 10-70, 10-65, 10-60, 10-55, 10-50, 10-45, 10-40, 10-35, 10-34.5, 10-34, 10-33.5, 10-33, 10-32.5, 10-32, 10-31.5, 10-31, 10-30.5, 10-30, 10-29.5, 10-29, 10-28.5, 10-28, 10-27.5, 10-27, 10-26.5, 10-26, 10-25.5, 10-25, 10-24.5, 10-24, 10-23.5, 10-23, 10-22.5, 10-22, 10-21.5, 10-21, 10-20.5, 10-20, 10-19.5, 10-19, 10-18.5, 10-18, 10-17.5, 10-17, 10-16.5, 10-16, 10-15.5, 10-15, 10-14.5, 10-14, 10-13.5, 10-13, 10-12.5, 10-12, 10-11.5, 10-11, 10-10.5, 10.5-500, 10.5-450, 10.5-400, 10.5-350, 10.5-300, 10.5-250, 10.5-200, 10.5-150, 10.5-100, 10.5-95, 10.5-90, 10.5-85, 10.5-80, 10.5-75, 10.5-70, 10.5-70, 10.5-65, 10.5-60, 10.5-55, 10.5-50, 10.5-45, 10.5-40, 10.5-35, 10.5-34.5, 10.5-34, 10.5-33.5, 10.5-33, 10.5-32.5, 10.5-32, 10.5-31.5, 10.5-31, 10.5-30.5, 10.5-30, 10.5-29.5, 10.5-29, 10.5-28.5, 10.5-28, 10.5-27.5, 10.5-27, 10.5-26.5, 10.5-26, 10.5-25.5, 10.5-25, 10.5-24.5, 10.5-24, 10.5-23.5, 10.5-23, 10.5-22.5, 10.5-22, 10.5-21.5, 10.5-21, 10.5-20.5, 10.5-20, 10.5-19.5, 10.5-19, 10.5-18.5, 10.5-18, 10.5-17.5, 10.5-17, 10.5-16.5, 10.5-16, 10.5-15.5, 10.5-15, 10.5-14.5, 10.5-14, 10.5-13.5, 10.5-13, 10.5-12.5, 10.5-12, 10.5-11.5, 10.5-11, 11-500, 11-450, 11-400, 11-350, 11-300, 11-250, 11-200, 11-150, 11-100, 11-95, 11-90, 11-85, 11-80, 11-75, 11-70, 11-70, 11-65, 11-60, 11-55, 11-50, 11-45, 11-40, 11-35, 11-34.5, 11-34, 11-33.5, 11-33, 11-32.5, 11-32, 11-31.5, 11-31, 11-30.5, 11-30, 11-29.5, 11-29, 11-28.5, 11-28, 11-27.5, 11-27, 11-26.5, 11-26, 11-25.5, 11-25, 11-24.5, 11-24, 11-23.5, 11-23, 11-22.5, 11-22, 11-21.5, 11-21, 11-20.5, 11-20, 11-19.5, 11-19, 11-18.5, 11-18, 11-17.5, 11-17, 11-16.5, 11-16, 11-15.5, 11-15, 11-14.5, 11-14, 11-13.5, 11-13, 11-12.5, 11-12, 11-11.5, 11.5-500, 11.5-450, 11.5-400, 11.5-350, 11.5-300, 11.5-250, 11.5-200, 11.5-150, 11.5-100, 11.5-95, 11.5-90, 11.5-85, 11.5-80, 11.5-75, 11.5-70, 11.5-70, 11.5-65, 11.5-60, 11.5-55, 11.5-50, 11.5-45, 11.5-40, 11.5-35, 11.5-34.5, 11.5-34, 11.5-33.5, 11.5-33, 11.5-32.5, 11.5-32, 11.5-31.5, 11.5-31, 11.5-30.5, 11.5-30, 11.5-29.5, 11.5-29, 11.5-28.5, 11.5-28, 11.5-27.5, 11.5-27, 11.5-26.5, 11.5-26, 11.5-25.5, 11.5-25, 11.5-24.5, 11.5-24, 11.5-23.5, 11.5-23, 11.5-22.5, 11.5-22, 11.5-21.5, 11.5-21, 11.5-20.5, 11.5-20, 11.5-19.5, 11.5-19, 11.5-18.5, 11.5-18, 11.5-17.5, 11.5-17, 11.5-16.5, 11.5-16, 11.5-15.5, 11.5-15, 11.5-14.5, 11.5-14, 11.5-13.5, 11.5-13, 11.5-12.5, 11.5-12, 12-500, 12-450, 12-400, 12-350, 12-300, 12-250, 12-200, 12-150, 12-100, 12-95, 12-90, 12-85, 12-80, 12-75, 12-70, 12-70, 12-65, 12-60, 12-55, 12-50, 12-45, 12-40, 12-35, 12-34.5, 12-34, 12-33.5, 12-33, 12-32.5, 12-32, 12-31.5, 12-31, 12-30.5, 12-30, 12-29.5, 12-29, 12-28.5, 12-28, 12-27.5, 12-27, 12-26.5, 12-26, 12-25.5, 12-25, 12-24.5, 12-24, 12-23.5, 12-23, 12-22.5, 12-22, 12-21.5, 12-21, 12-20.5, 12-20, 12-19.5, 12-19, 12-18.5, 12-18, 12-17.5, 12-17, 12-16.5, 12-16, 12-15.5, 12-15, 12-14.5, 12-14, 12-13.5, 12-13, 12-12.5, 12.5-500, 12.5-450, 12.5-400, 12.5-350, 12.5-300, 12.5-250, 12.5-200, 12.5-150, 12.5-100, 12.5-95, 12.5-90, 12.5-85, 12.5-80, 12.5-75, 12.5-70, 12.5-70, 12.5-65, 12.5-60, 12.5-55, 12.5-50, 12.5-45, 12.5-40, 12.5-35, 12.5-34.5, 12.5-34, 12.5-33.5, 12.5-33, 12.5-32.5, 12.5-32, 12.5-31.5, 12.5-31, 12.5-30.5, 12.5-30, 12.5-29.5, 12.5-29, 12.5-28.5, 12.5-28, 12.5-27.5, 12.5-27, 12.5-26.5, 12.5-26, 12.5-25.5, 12.5-25, 12.5-24.5, 12.5-24, 12.5-23.5, 12.5-23, 12.5-22.5, 12.5-22, 12.5-21.5, 12.5-21, 12.5-20.5, 12.5-20, 12.5-19.5, 12.5-19, 12.5-18.5, 12.5-18, 12.5-17.5, 12.5-17, 12.5-16.5, 12.5-16, 12.5-15.5, 12.5-15, 12.5-14.5, 12.5-14, 12.5-13.5, 12.5-13, 13-500, 13-450, 13-400, 13-350, 13-300, 13-250, 13-200, 13-150, 13-100, 13-95, 13-90, 13-85, 13-80, 13-75, 13-70, 13-70, 13-65, 13-60, 13-55, 13-50, 13-45, 13-40, 13-35, 13-34.5, 13-34, 13-33.5, 13-33, 13-32.5, 13-32, 13-31.5, 13-31, 13-30.5, 13-30, 13-29.5, 13-29, 13-28.5, 13-28, 13-27.5, 13-27, 13-26.5, 13-26, 13-25.5, 13-25, 13-24.5, 13-24, 13-23.5, 13-23, 13-22.5, 13-22, 13-21.5, 13-21, 13-20.5, 13-20, 13-19.5, 13-19, 13-18.5, 13-18, 13-17.5, 13-17, 13-16.5, 13-16, 13-15.5, 13-15, 13-14.5, 13-14, 13-13.5, 13.5-500, 13.5-450, 13.5-400, 13.5-350, 13.5-300, 13.5-250, 13.5-200, 13.5-150, 13.5-100, 13.5-95, 13.5-90, 13.5-85, 13.5-80, 13.5-75, 13.5-70, 13.5-70, 13.5-65, 13.5-60, 13.5-55, 13.5-50, 13.5-45, 13.5-40, 13.5-35, 13.5-34.5, 13.5-34, 13.5-33.5, 13.5-33, 13.5-32.5, 13.5-32, 13.5-31.5, 13.5-31, 13.5-30.5, 13.5-30, 13.5-29.5, 13.5-29, 13.5-28.5, 13.5-28, 13.5-27.5, 13.5-27, 13.5-26.5, 13.5-26, 13.5-25.5, 13.5-25, 13.5-24.5, 13.5-24, 13.5-23.5, 13.5-23, 13.5-22.5, 13.5-22, 13.5-21.5, 13.5-21, 13.5-20.5, 13.5-20, 13.5-19.5, 13.5-19, 13.5-18.5, 13.5-18, 13.5-17.5, 13.5-17, 13.5-16.5, 13.5-16, 13.5-15.5, 13.5-15, 13.5-14.5, 13.5-14, 14-500, 14-450, 14-400, 14-350, 14-300, 14-250, 14-200, 14-150, 14-100, 14-95, 14-90, 14-85, 14-80, 14-75, 14-70, 14-70, 14-65, 14-60, 14-55, 14-50, 14-45, 14-40, 14-35, 14-34.5, 14-34, 14-33.5, 14-33, 14-32.5, 14-32, 14-31.5, 14-31, 14-30.5, 14-30, 14-29.5, 14-29, 14-28.5, 14-28, 14-27.5, 14-27, 14-26.5, 14-26, 14-25.5, 14-25, 14-24.5, 14-24, 14-23.5, 14-23, 14-22.5, 14-22, 14-21.5, 14-21, 14-20.5, 14-20, 14-19.5, 14-19, 14-18.5, 14-18, 14-17.5, 14-17, 14-16.5, 14-16, 14-15.5, 14-15, 14-14.5, 14.5-500, 14.5-450, 14.5-400, 14.5-350, 14.5-300, 14.5-250, 14.5-200, 14.5-150, 14.5-100, 14.5-95, 14.5-90, 14.5-85, 14.5-80, 14.5-75, 14.5-70, 14.5-70, 14.5-65, 14.5-60, 14.5-55, 14.5-50, 14.5-45, 14.5-40, 14.5-35, 14.5-34.5, 14.5-34, 14.5-33.5, 14.5-33, 14.5-32.5, 14.5-32, 14.5-31.5, 14.5-31, 14.5-30.5, 14.5-30, 14.5-29.5, 14.5-29, 14.5-28.5, 14.5-28, 14.5-27.5, 14.5-27, 14.5-26.5, 14.5-26, 14.5-25.5, 14.5-25, 14.5-24.5, 14.5-24, 14.5-23.5, 14.5-23, 14.5-22.5, 14.5-22, 14.5-21.5, 14.5-21, 14.5-20.5, 14.5-20, 14.5-19.5, 14.5-19, 14.5-18.5, 14.5-18, 14.5-17.5, 14.5-17, 14.5-16.5, 14.5-16, 14.5-15.5, 14.5-15, 15-500, 15-450, 15-400, 15-350, 15-300, 15-250, 15-200, 15-150, 15-100, 15-95, 15-90, 15-85, 15-80, 15-75, 15-70, 15-70, 15-65, 15-60, 15-55, 15-50, 15-45, 15-40, 15-35, 15-34.5, 15-34, 15-33.5, 15-33, 15-32.5, 15-32, 15-31.5, 15-31, 15-30.5, 15-30, 15-29.5, 15-29, 15-28.5, 15-28, 15-27.5, 15-27, 15-26.5, 15-26, 15-25.5, 15-25, 15-24.5, 15-24, 15-23.5, 15-23, 15-22.5, 15-22, 15-21.5, 15-21, 15-20.5, 15-20, 15-19.5, 15-19, 15-18.5, 15-18, 15-17.5, 15-17, 15-16.5, 15-16, 15-15.5, 15.5-500, 15.5-450, 15.5-400, 15.5-350, 15.5-300, 15.5-250, 15.5-200, 15.5-150, 15.5-100, 15.5-95, 15.5-90, 15.5-85, 15.5-80, 15.5-75, 15.5-70, 15.5-70, 15.5-65, 15.5-60, 15.5-55, 15.5-50, 15.5-45, 15.5-40, 15.5-35, 15.5-34.5, 15.5-34, 15.5-33.5, 15.5-33, 15.5-32.5, 15.5-32, 15.5-31.5, 15.5-31, 15.5-30.5, 15.5-30, 15.5-29.5, 15.5-29, 15.5-28.5, 15.5-28, 15.5-27.5, 15.5-27, 15.5-26.5, 15.5-26, 15.5-25.5, 15.5-25, 15.5-24.5, 15.5-24, 15.5-23.5, 15.5-23, 15.5-22.5, 15.5-22, 15.5-21.5, 15.5-21, 15.5-20.5, 15.5-20, 15.5-19.5, 15.5-19, 15.5-18.5, 15.5-18, 15.5-17.5, 15.5-17, 15.5-16.5, or 15.5-16 micrometers. Each possibility represents a separate embodiment of the invention. In some embodiments, the average particle size in the powder is between 0.1 and 500 micrometers. In some embodiments, size is diameter. Powders can be produced by methods well known in the art, such as, applying sheering forces, performing homogenization, no pressure homogenization, pressure homogenization, high pressure homogenization, wet milling, spray drying, freeze drying, fluidizer, and dry milling. These methods produce average particle sizes such as are recited herein.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/of” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Production of New Betacyanin Pigment Composition

The Inventors disclosed in International Patent Publication WO2017/122189 methods of producing pigment compositions comprising betalains by transient expression of exogenous genes of the betalain production pathway in yeast. Herein, this work has been built upon to produce superior betalain containing pigment compositions. In specific, these compositions comprise very high concentrations of betacyanin pigment molecules, contain both betanin and betanidin (with a very high betanin to betanidin ratio) and have unique color expression.

Saccharomyces cerevisiae strain BY4742 (S288C derivative) was used for initial betalain production. Rather than transient expression of the genes of the betalain pathway as had been done previously, the exogenous genes were now added to the yeast cells by genomic integration. Genomic integration allowed for a more precise control of the expression of the various genes. Five different lines and the exogenous genes they express are described in FIG. 5. All the stains expressed a tyrosine hydroxylase/L-DOPA oxidase (CYP76AD1), a DOPA 4,5-dioxygenase (MjDOD), an NADPH cytochrome reductase (ATR2) and a cyclo-DOPA-5-O-glucosyltransferase (MjcDOPA5GT). Four of the strains also expressed a betanidin-5-O-glucosyltransferase (from Dorotheanthus bellidiformis, Db5GT). Since the betalain products are secreted, no extraction is required, and the recovery process is initiated by biomass clearance, followed by concentration and stabilization of the secreted betalain pigments. When the secreted pigments were analyzed by HPLC, it was determined that all strains produced and secreted both betanin and betanidin. This is in contrast to International Patent Publication WO2017/122189 in which only betanidin was produced. Betanidin is an intermediate molecule, that must undergo glycosyl transfer (glycosylation) to produce betanin. As this previous work did not express any glycosyltransferases in the yeast, only betanidin was produced and secreted. This is also in contrast to natural beetroot extracts which contain only betanin (see FIG. 2). Betanin is the natural final product of the pathway and so is abundant in natural beetroot. Indeed, the pathway is so efficient in the beet that beetroot extracts and beetroot juice are totally devoid of detectable levels of betanidin (FIG. 2). It is hypothesized that the glycosyl transfer is less efficient in yeast, possibly due to a limiting free pool of UDP-glucose (the substrate for the glycosyltransferase) and so high amounts of betanidin build up in the yeast cells and are secreted.

It was noted that by increasing the copy number of the various genes of the pathway the total yield of betacyanins could be increased (FIG. 5). A doubling of the components of the pathway produced an increase in yield of about 10-100%. Notably, at these higher yields the ratio of betanidin to betanin held steady. However, at these high levels of betalain synthesis, the pool of free UDP-glucose was hypothesized to be limiting with respect to how much betanin could be produced. Therefore, in order to increase the yield of betanin (increase the efficiency of the glycosyl transfer) more copies of the glycosyltransferase genes were introduced while keeping the other genes of the pathways unchanged. Further, additional genes or gene knockouts were introduced into the yeast to increase free UDP-glucose levels. As can be seen in FIG. 5 these modifications did indeed result in higher amounts of betanin and lower amounts of betanidin. Nevertheless, the production of betanin is still much more inefficient than in beet and so considerable amounts of betanidin are still produced.

The liquid preparation of the product contained at least 0.04% (w/w, i.e., 0.4 gr/l) betacyanin and the powder preparation contained at least 0.05% (w/w, i.e., 0.5 gr/Kg) betacyanin with a chromatographic purity of ≥80% at 534-540 nm (range of λmax for most betacyanin), meaning that over 80% of the relative peak area is attributed to betacyanin pigments. These yields are greatly increased as compared to the compositions produced in WO2017/122189. In that work only liquid formulations were produced and the highest concentration achieved was only 20 mg/l.

In the fermentation Process, the production organism is grown under submerged fed-batch, batch or continuous fermentation for the production of the pigments. Working cell banks are prepared from the master cell bank after being tested for microbial purity, specific growth rate, final biomass (dry cell weight) and production yields, prior to the fermentation process. The microbial purity is also periodically validated by microscopy and microbial culture of the fermentation broth. The process parameters including pH, temperature, agitation, and dissolved oxygen are routinely controlled and monitored throughout the process. The broth containing the product is harvested by removing the production organism from the fermentation broth at the end of the fermentation process using filtration, microfiltration, centrifugation, filter press or any other method. A fully chemically defined medium was used for this process. In previous works a commercial synthetic medium was used (standard SD media).

In the downstream processes (DSP), a series of ultrafiltration and/or nanofiltration (with or without diafiltrations) steps or other suitable processes are used to purify and concentrate the pigment composition. The production can also include an additional step of water evaporation to concentrate the material for a liquid preparation. The liquid preparation can be dried to a powder form using a dryer (spray/freeze) or a fluidizer (fluidized bed reactor). These DSP were fully absent from the Inventors' previous work.

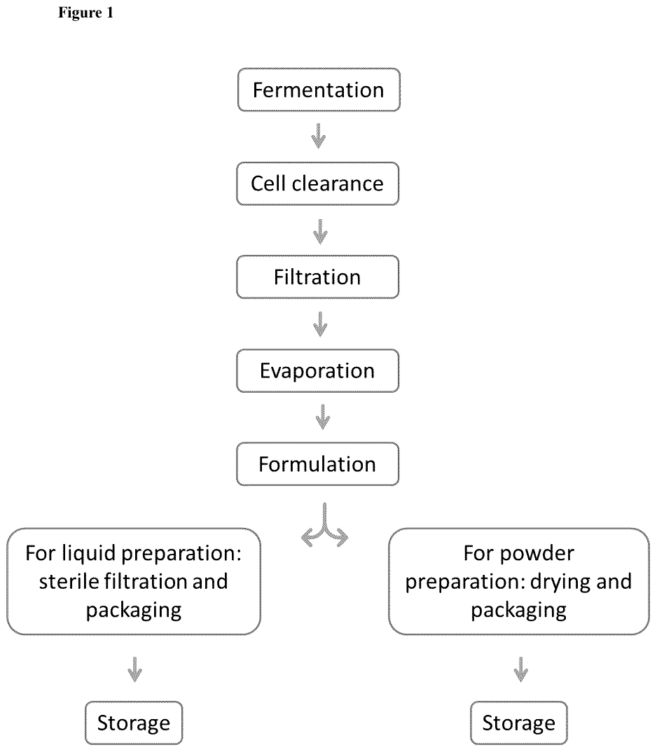

A schematic overview of the manufacturing process for the pigment composition is presented in FIG. 1. After downstream processing, yields of greater than 4 gr/L betacyanins were achieved (FIG. 5). This is highly significant, as 4 gr/L is considered the minimum concentration needed for a commercial composition. Extracts that are insufficiently concentrated cannot be easily added to foods without altering taste/appearance and generating unwanted bulk. In contrast, Tinggaard Thomsen et al., which describes an optimized yeast strain for betalain production produced at best 1.2-1.3 gr/L betacyanins.

### Example 2: Comparison of New Formulation to Known Beetroot Compositions

Beetroot (Beta vulgaris) is the only commercially available source of betacyanin pigments, produced by physical and/or chemical extraction of the pigments from the red beets. The new formulation produced herein was termed PhytoPurple and was compared to two known commercially available beetroot compositions in addition to fresh beetroot juice. Five different batches of PhytoPurple were produced that were each slightly different (see FIG. 5). One of them, Pn32-F09 was examined in depth and compared to commercially available beetroot extracts and fresh beetroot juice. The compositions were analyzed by liquid chromatography-mass spectrometry (LC-MS) and various aspects of the compositions were quantified. This includes their elemental composition, nutritional values, and pigment composition (FIG. 2). Firstly, the PhytoPurple composition was completely devoid of beet impurities such as geosmin and nitrates. Similarly, it contained a much lower caloric content than beet extracts and was essentially devoid of simple sugars unlike beet products.

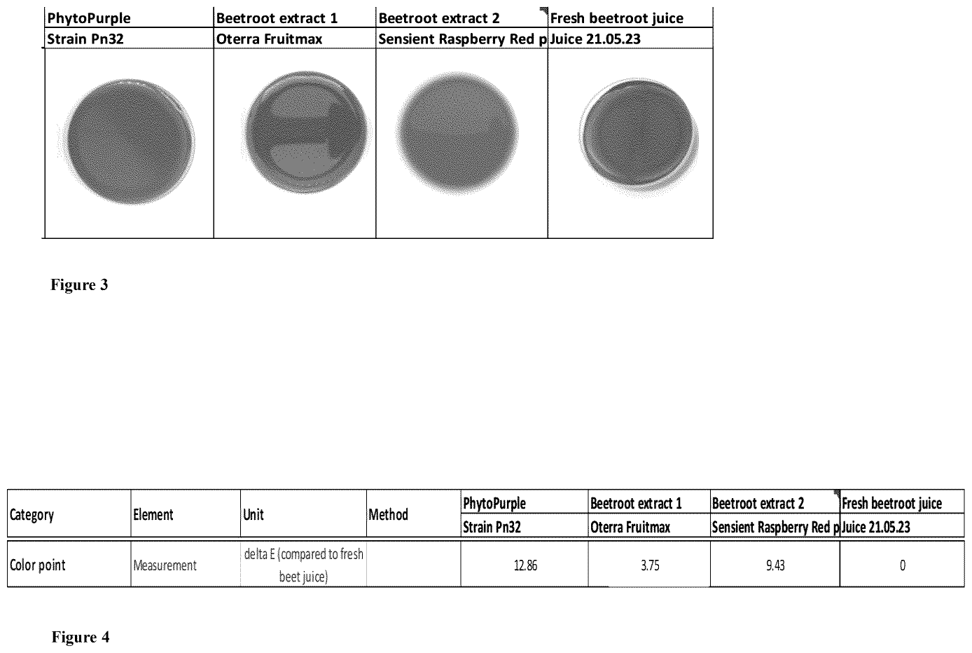

The betacyanins produced were also very different than what is found in beet. The differences in pigment composition were quantified not only by liquid chromatography (FIG. 2), but also by spectrophotometry (FIG. 3). Commercial beet extracts and juice contained only betanin with no measurable amounts of betanidin. Further, the commercial beetroot extracts had very high levels of iso-betanin as compared to betanin, although the levels were lower in the beetroot juice. In contrast, the PhytoPurple composition contained both betanin and betanidin.

Measurements of L*, a*, b* values of the CIELAB color space by chromameter and comparison of these values to other compositions also found the color of the new compositions to be distinct (score of >2.5) (FIG. 4). This demonstrates that the pigment composition produced by genomic integration of multiple copies of the betalain synthesis pathway into yeast is distinct from the pigment composition produced by beet. Further, the color difference caused by the inclusion of betanidin is sufficiently pronounced that it can be observed by eye. Even small changes in the amounts of betanin and betanidin can affect the final color and the instant system provides a means for carefully controlling this ratio.

### Example 3: Production of Betacyanin Pigment Composition with Very High Levels of Betanin/Iso-Betanin

In an effort to tilt production even more toward betanin instead of betanidin, further genetic modifications were made to the bacteria. Three new strains were generated in the S. cerevisiae strain CEN.PK113-7D (FIG. 6). These strains varied the number of CYP76AD1 gene insertions, used ATR2 as the only reductase added, though an increased number of copies of ATR2 were added, and tested the addition of plasma membrane transporter QDR2. Further, all three strains knocked out expression of 3 genes from the galactose metabolism pathway: gal80, gal1 and gal10. Finally, the promoters linked to these genes were switched to the very strongly active Gal1/10 promoter. All three strains produced very high levels of betanin/iso-betanin, in particular strains CPN7 and CPN8 which included the modifications of the UDP-glucose pathway previously incorporated into Pn32. 98-99% betanin was successfully produced in strains CPN7 and CPN8, with very high titers at the end of fermentation. No betaxanthin was detected in any of the new strains.

The differences in pigment composition were quantified not only by liquid chromatography (FIG. 6), but also by spectrophotometry (FIG. 7). Measurements of L*, a*, b* values of the CIELAB color space by chromameter (Color Point) were also performed for these compositions (FIG. 7). Commercial beet extracts and juice contained only betanin with no measurable amounts of betanidin. Therefore, not surprisingly, CPN-6 which contained the most betanidin was most clearly a different color than the beet compositions. Even CPN-7 which had only about 2% betanidin was clearly visually distinct from beetroot extracts and beet juice (FIG. 3). CPN-8 more closely resembled the beetroot extracts but was still clearly distinct in color as compared to the beet juice. These observations by eye corresponded to the measurements with Color Point.

Importantly, these three strains again produced a pigment with very low sugar content (less than 4 gr/100 gram) and very low sodium content (lower than 50 mg/L) just as was observed for the Pn strains. Further, the various contaminants such as geosmin and nitrates found in naturally produced betalain pigment compositions (such as from beet) were not detected. In the final formulation, carriers and whey protein concentrate (WPC) were added for bulk, and tara gum was added as a thickener and stabilizer.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A pigment composition comprising betalain pigment molecules, wherein said betalain pigment molecules comprise betanin and betanidin, wherein at least 0.5% of said betalain pigment molecules are betanidin and at least 10% of said betalain pigment molecules are betanin.

2. (canceled)

3. (canceled)

4. (canceled)

5. (canceled)

6. The pigment composition of claim 5, wherein said betanidin is at least 10% of said betalain pigment molecules, is between 1% and 90% of said betalain pigment molecules or is between 10% and 75% of said betalain pigment molecules.

7. (canceled)

8. (canceled)

9. The pigment composition of claim 1, wherein said composition comprises less than 5% sugars by weight, optionally comprising less than 1% sugars by weight.

10. (canceled)

11. The pigment composition of claim 1, wherein said composition comprises at least one of:
a) less than 0.1 μg/kg geosmin, less than 0.01 g/L nitrates or both;
b) less than 10% dietary carbohydrates by weight;
c) less than 10% neobetanin;
d) less than 50 mg sodium per liter of composition; and
e) being essentially devoid of intact cells.

12. The pigment composition of claim 1, wherein said betalain pigment molecules further comprise iso-betanidin.

13. The pigment composition of claim 12, wherein a ratio of betanidin to iso-betanidin is at least 5:1.

14. (canceled)

15. (canceled)

16. (canceled)

17. (canceled)

18. The pigment composition of claim 1, wherein said betalain pigment molecules comprise betanin and iso-betanin and wherein a ratio of betanin to iso-betanin in said pigment composition is more than 7:1.

19. (canceled)

20. The pigment composition of claim 1, wherein said composition is a powder.

21. (canceled)

22. The pigment composition of claim 1, wherein less than 10% of said betalain pigment molecules are betaxanthins.

23. (canceled)

24. (canceled)

25. (canceled)

26. The pigment composition of claim 1, being a powder and comprising greater than 1.5 grams betalain pigment molecules per kilogram of composition.

27. (canceled)

28. A cell comprising at least one copy of an exogenous tyrosine hydroxylase gene, at least one copy of an exogenous L-DOPA oxidase gene, at least one copy of an exogenous DOPA 4,5-dioxygenase (DOD) gene, at least one copy of an exogenous NADPH cytochrome P450 reductase gene, and at least one copy of a cyclo-DOPA-5-O-glucosyltransferase gene integrated into the genome of the cell, optionally wherein said cell is a plant cell, a bacterial cell or a fungal cell.

29. The cell of claim 28, wherein at least one of:
a) said tyrosine hydroxylase gene and said L-DOPA oxidase gene are the same gene;
b) said tyrosine hydroxylase gene and said L-DOPA oxidase gene are CYP76AD1;
c) said DOD is Mirabilis jalapa DOD (MjDOD);
d) said reductase is NADPH-cytochrome P450 reductase 2 (ATR2) or NADPH-cytochrome P450 reductase 1 (ATR1);
e) said cyclo-DOPA-5-O-glucosyltransferase is a Mirabilis jalapa cyclo-DOPA-5-O-glucosyltransferase (MjcDOPA5GT), a Chenopodium quinoa cyclo-DOPA-5-O-glucosyltransferase (CqcDOPA5GT) or both.

30. The cell of claim 29, wherein said tyrosine hydroxylase gene and said L-DOPA oxidase gene are CYP76AD1; said DOD is Mirabilis jalapa DOD (MjDOD); said reductase is NADPH cytochrome P450 reductase 2 (ATR2) or NADPH cytochrome P450 reductase 1 (ATR1); and said cyclo-DOPA-5-O-glucosyltransferase is a Mirabilis jalapa cyclo-DOPA-5-O-glucosyltransferase (MjcDOPA5GT), a Chenopodium quinoa cyclo-DOPA-5-O-glucosyltransferase (CqcDOPA5GT) or both.

31. (canceled)

32. (canceled)

33. (canceled)

34. The cell of claim 28,
a) comprising at least two copies of said exogenous tyrosine hydroxylase gene, at least two copies of said exogenous L-DOPA oxidase gene, at least two copies of said exogenous reductase gene and at least two copies of said exogenous glycosyl transferase gene integrated in the genome of said cell, optionally comprising at least two copies of said exogenous DOD gene;
b) comprising 3 copies of a CYP76AD1 gene, 1 copy of a DOD gene, 3 copies of an NADPH cytochrome P450 reductase gene and 3 copies of a cyclo-DOPA-5-O-glucosyltransferase gene integrated into the genome of said cell; or
c) comprising 5 copies of a CYP76AD1 gene, 2 or 3 copies of a DOD gene, 4 copies of an NADPH cytochrome P450 reductase gene and 3 copies of a cyclo-DOPA-5-O-glucosyltransferase gene integrated into the genome of said cell.

35. (canceled)

36. (canceled)

37. (canceled)

38. The cell of claim 28, further comprising at least one copy of an exogenous Cytochrome b5 reductase (Cyb5) gene integrated into the genome of said yeast cell.

39. The cell of claim 28, further comprising at least one copy of an exogenous betanidin-5-O-glucosyltransferase gene integrated into the genome of said yeast cell; optionally wherein said betanidin-5-0-glucosyltransferase is a Dorotheanthus bellidiformis betanidin-5-O-glucosyltransferase (Db5GT).

40. The cell of claim 28, further comprising at least one copy of an exogenous gene integrated into the genome of the cell, at least one knockout of an endogenous gene in the cell or both that result in increased production of UDP-glucose, wherein said exogenous gene is selected from: UGP1, PGM2, URA6 and YNK1: said endogenous gene is selected from exg1 and ynd1; or both.

41. (canceled)

42. The cell of claim 40, comprising at least one exogenous copy of each of UGP1, PGM2, URA6 and YNK1 integrated into the genome of the cell and knockout of endogenous exg1 and endogenous ynd1 in the cell.

43. The cell of claim 28, further comprising at least one copy of an exogenous plasma membrane transporter gene integrated into the genome of the cell; optionally wherein said plasma membrane transporter is a yeast plasma membrane transporter, optionally wherein said yeast plasma membrane transporter is QDR2.

44. (canceled)

45. The cell of claim 28, comprising knockdown of endogenous gal80, gal1 and gal10.

46. (canceled)

47. (canceled)

48. (canceled)
